(19) United States
(12) Patent Application Publication
(10) Pub. No.: US 2021/0166813 A1
(43) Pub. Date: Jun. 3, 2021

(54) SYSTEMS AND METHODS FOR EVALUATING LONGITUDINAL BIOLOGICAL FEATURE DATA

(71) Applicant: GRAIL, Inc., Menlo Park, CA (US)

(72) Inventors: M. Cyrus Maher, San Mateo, CA (US); Alex Aravanis, San Mateo, CA (US); Angela Lai, Los Altos Hills, CA (US); Oliver Claude Venn, San Francisco, CA (US); Richard Rava, Redwood City, CA (US); Jing Xiang, San Carlos, CA (US); Joseph Marcus, Venice, CA (US)

(21) Appl. No.: 17/105,175

(22) Filed: Nov. 25, 2020

Related U.S. Application Data
(60) Provisional application No. 62/941,012, filed on Nov. 27, 2019.

(51) Int. Cl. G16H 50/20; G16B 20/00; G16B 5/00; G16B 40/30; G16H 50/70; G16H 50/30

(52) U.S. Cl. CPC G16H 50/20; G16B 20/00; G16H 50/30; G16B 40/30; G16H 50/70; G16B 5/00

(57) ABSTRACT

Systems and methods are provided for determining whether a test subject has a disease condition. In one aspect, the method includes determining at least first and second genotypic data constructs for a test subject, formed from data collected from first and second sample from the subject, respectively, at different times. The first and second genotypic data constructs are inputted into a model for the disease condition, thereby generating first and second model score sets for the disease condition, respectively. A test delta score set is determined based on a difference between the first and second model score sets. The test delta score set is evaluated against a plurality of reference delta score sets, to determine the disease condition of the test subject, where each reference delta score set is for a respective reference subject in a plurality of reference subjects.

### CROSS-REFERENCE TO RELATED APPLICATIONS

This application claims the benefit of U.S. Provisional Application No. 62/941,012, filed on Nov. 27, 2019, which is expressly incorporated herein by reference in its entirety for all purposes.

### TECHNICAL FIELD

This disclosure relates to methods for evaluating the disease status of a subject based on changes in genotypic characteristics of the subject over time.

### BACKGROUND

Cancer represents a prominent worldwide public health problem. The United States alone in 2015 had a total of 1,658,370 cases reported. Screening programs and early diagnosis have an important impact in improving disease-free survival and reducing mortality in cancer patients. For example, early screening of colorectal cancer (CRC) has led to almost a 50% decrease in CRC incidence and mortality in the U.S. This reduction is consistent with stage-dependent survival rates for the cancer, which decrease from 94% in stage 1 CRC to 11% in stage 4 CRC. However, there are two major challenges with early cancer detection: patient compliance and poor sensitivity.

Advantageously, increasing knowledge of the molecular pathogenesis of cancer and the rapid development of next generation sequencing techniques are advancing the study of early molecular alterations involved in cancer development in body fluids. Specific genetic and epigenetic alterations associated with such cancer development are found in cell-free DNA (cfDNA) in plasma, serum, and urine. Such alterations can potentially be used as diagnostic biomarkers for several types of cancers. Advantageously, non-invasive sampling methods, such as so-called ‘liquid biopsies,’ can foster patient compliance, as they are easier, quicker, and less expensive to perform.

Cell-free DNA (cfDNA) can be found in serum, plasma, urine, and other body fluids enabling the ‘liquid biopsy,’ which represents a snapshot of the genomic makeup of many different tissues in the subject, including diseased tissues. cfDNA originates from necrotic or apoptotic cells, and it is generally released by all types of cells. cfDNA contains specific tumor-related alterations, such as mutations, methylation, and copy number variations (CNVs), thus comprising circulating tumor DNA (ctDNA).

However, because cfDNA represents DNA released from a wide range of tissues, including healthy tissues and white blood cells undergoing hematopoiesis, the challenge remains to be able to differentiate the signal originating from a disease tissue, such as cancer, from signals originating from germline cells. In fact, in most cancer patients, the majority of cfDNA is from healthy cells, e.g., greater than 80%, 90%, 95%, or more. cfDNA signals can be enriched, for example, bioinformatically by identifying variant alleles having allele fractions that do not adhere to typical 1:1 ratios, as seen for heterozygous alleles in the germline. cfDNA signals can also be enriched based on the size of the cfDNA being sequenced, because it has been observed that cfDNA originating from cancerous tumor is, on average, shorter in length than cfDNA originating from germline cells.

Unfortunately, to date, the majority of cfDNA diagnostic studies are focused on advanced tumor stages. The application of cfDNA-based diagnostic assays for identification of early malignant disease stages is less well documented. Although early stage cancer detection works on the same principals as later stage cancer detection, there are several impediments that are unique to early stage detection. These include lower frequency and volume of aberrations, potentially confounding phenomena such as clonal expansions of non-tumorous tissues or the accumulation of cancer-associated mutations with age, and the incomplete insight into driver alterations.

In blood, apoptosis is a frequent event that determines the amount of cfDNA. In cancer patients, however, the amount of cfDNA can also be influenced by necrosis. Since apoptosis seems to be the main release mechanism, circulating cfDNA has a size distribution that reveals an enrichment in short fragments of about 167 bp, corresponding to nucleosomes generated by apoptotic cells.

### SUMMARY

Generally, the systems and methods described herein can facilitate earlier detection of a disease state than is possible using conventional classification methods, by accounting for individualized variance in the subject's biological signatures. Conventional methods for classifying the disease status of a subject can involve taking a snapshot of one or more biological signatures of the subject at a single time point, and evaluating the subject's information against a predetermined disease profile or trained classifier. While this approach is sufficient for identifying the presence of a disease when it has sufficiently progressed in a subject, it typically cannot allow for confident detection pre-disease states or even early stages of the disease. For instance, several classifiers have been developed for diagnosing cancer in a subject by interrogating sequence reads of cell-free DNA (cfDNA) isolated from the blood plasma of the subject. However, because blood plasma contains cfDNA from healthy germline cells and hematopoietic cells, these classifiers use a minimum amount of circulating tumor DNA (ctDNA), referred to as a minimum tumor fraction, that is present in the blood plasma in order to detect a cancerous signature in the cfDNA sequence reads. However, because there is a strong correlation between the stage at which a disease is diagnosed and treatment outcomes, more sensitive methods that can identify the presence of a disease at an earlier stage are needed.

Advantageously, the present disclosure provides such methods for earlier disease identification, at least in part, by interrogating the changes in a subject's biological signatures over time, as opposed to at a single time point. Specifically, by using data across multiple biological samples from a subject over time, personalized variance in biological characteristics of the subject can be accounted for when monitoring for a disease state.

In one aspect, the present disclosure provides a method for determining the disease state of a subject by comparing a change, over time, in a modeled probability that the subject has the disease state to a population distribution of changes in modeled probability over time. In some embodiments, the method includes determining a first genotypic data construct for the test subject, the first genotypic data construct including values for a plurality of genotypic characteristics based on a first plurality of sequence reads, in electronic form, of a first plurality of nucleic acid molecules in a first biological sample obtained from the test subject at a first test time point. The method can include inputting the first genotypic data construct into a model for the disease condition, thereby generating a first model score set for the disease condition. The method can include determining a second genotypic data construct for the test subject, the second genotypic data construct including values for the plurality of genotypic characteristics based on a second plurality of sequence reads, in electronic form, of a second plurality of nucleic acid molecules in a second biological sample obtained from the test subject at a second test time point occurring after the first test time point. The method can include inputting the second genotypic data construct into the model, thereby generating a second model score set for the disease condition. The method can include determining a test delta score set based on a difference between the first and second model score set. Then the method can include evaluating the test delta score set against a plurality of reference delta score sets, thereby determining the disease condition of the test subject, where each reference delta score set in the plurality of reference delta scores sets is for a respective reference subject in a plurality of reference subjects.

In another aspect, the present disclosure provides a method for determining the disease state of a subject by evaluating changes, over time, in a modeled probability that the subject has the disease state using a temporal trend test. In some embodiments, the method includes determining, for each respective test time point in a plurality of test time points, a corresponding genotypic data construct for the test subject, the corresponding genotypic data construct including values for a plurality of genotypic characteristics based on a corresponding plurality of sequence reads, in electronic form, of a corresponding plurality of nucleic acid molecules in a corresponding biological sample obtained from the test subject at the respective test time point. The method can include inputting the corresponding genotypic data construct into a model for the disease condition (which is described separately herein) to generate a corresponding time stamped model score set for the disease condition at the respective test time point, thereby obtaining a plurality of time stamped test model score sets for the test subject, where each respective time stamped test model score set is coupled to a different test time point in the plurality of test time points. The method can include fitting the plurality of time stamped test model score sets with a temporal trend test, thereby obtaining a test trend parameter set for the test subject. The method can include evaluating the test trend parameter set for the test subject against a plurality of reference trend parameter sets for a plurality of reference subjects thereby determining the disease condition of the test subject, where each respective reference trend parameter set in the plurality of reference trend parameter sets is for a corresponding reference subject in the plurality of reference subjects.

The method can include creating a classifier based on data from all time-points to leverage all the time-points at once to learn disease conditions rather than applying a classifier marginally to each time-point (e.g., applying a pre-trained single time-point classifier to test samples collected from multiple time-points) and post-hoc analyzing model scores with temporal information (e.g., analyzing a significant trend or difference in cancer probabilities/scores with respect to a distribution of reference delta scores). For example, a joint model for detecting disease conditions (e.g., cancer signals) through time can be created. The joint model can be a multiple time-point classifier which is trained and tested on time-series data (e.g., time-series genotypic data construct). The joint model can improve the inference or results of the cancer probability and overall trend because data (e.g., the time-series data) is shared across multiple time-points. The joint model can include an asymptotic dimension for time space and can be trained jointly both for time space (e.g., time-series data) and feature space (e.g., other genotypic data constructs). In this situation, the joint model can include information that a genotypic data construct contributing to a cancer can be time-variant. The input to the multiple time-point classifier can include genotypic data construct (e.g., genomic features) and disease conditions (e.g., output-labels for cancer or non-cancer or tissue of origins) measured at two or more time points, and the multiple time-point classifier can include a logit transformation of probability of cancer corresponding to each sample and time point. During the process of determining disease conditions for new samples, the genotypic data construct of the new samples from previous time points can be used to estimate cancer probabilities for later time points, and vice versa. The joint model can be further trained and applied to test examples for classification by thresholding the estimated cancer probabilities to make predictions about the test samples' cancer states at their corresponding time-points (e.g., the current time-point). The joint model can also forecast cancer probability trends in the future, with or without medical interventions, based on the rate of change in the estimated cancer probability. To better improve classification and provide interpretability, different regularization approaches through probabilistic models or penalties can be used, such as encouraging the latent cancer probabilities to smoothly evolve through time, or enforcing a monotonic increase in cancer probability with stage.

### INCORPORATION BY REFERENCE

All publications, patents, and patent applications herein are incorporated by reference in their entireties. In the event of a conflict between a term herein and a term in an incorporated reference, the term herein controls.

### BRIEF DESCRIPTION OF THE DRAWINGS

The implementations disclosed herein are illustrated by way of example, and not by way of limitation, in the figures of the accompanying drawings. Like reference numerals refer to corresponding parts throughout the several views of the drawings.

FIGS. 1A and 1B collectively illustrate a block diagram for an example of a computing system for determining the disease state of a subject, in accordance with various embodiments of the present disclosure.

FIG. 2 illustrates an example of a workflow for determining the disease state of a subject, in accordance with various embodiments of the present disclosure.

FIGS. 3A, 3B, 3C, 3D, 3E, 3F, and 3G collectively illustrate an example process for determining the disease state of a subject, in accordance with various embodiments of the present disclosure.

FIGS. 4A, 4B, 4C, 4D, 4E, and 4F collectively illustrate an example process for determining the disease state of a subject, in accordance with various embodiments of the present disclosure.

FIGS. 5A, 5B, and 5C illustrate changes in cancer probabilities for a series of in silico augmented normal samples, as described in Example 1.

FIG. 6 illustrates distributions of cancer probabilities calculated for samples from age-matched and young healthy subjects without cancer, using a copy number-based cancer classifier.

FIGS. 7A and 7B illustrate in silico regression of copy number variation data, between a tumor fraction of 0.0 and 1.0 (FIG. 7A), and examples of cancer probabilities calculated from three simulated tumor fraction series, as a function of tumor fraction (FIG. 7B).

FIG. 8 shows cancer probabilities generated for samples collected and amplified using five different techniques from eight healthy reference subjects.

FIG. 9 shows the sensitivity of various cancer detection models achieved for each cancer stage, as defined by simulated tumor fraction.

FIG. 10 illustrates the distribution of changes in cancer probabilities determined for individuals using a cfDNA-based methylation cancer classifier, between first and second time points spaced from 12 to 40 months apart.

FIG. 11 illustrates a plot of cancer probabilities determined for individuals using a cfDNA-based methylation cancer classifier at first (abscissa) and second (ordinate) time points spaced from 12 to 40 months apart.

FIG. 12 illustrates changes in cancer probabilities determined for individuals using a cfDNA-based methylation cancer classifier, between first and second time points spaced from 12 to 40 months apart, plotted as a function of the time period between blood draws.

FIG. 13 illustrates a plot of cancer probabilities determined for select individuals using a cfDNA-based methylation cancer classifier at first (abscissa) and second (ordinate) time points spaced from 12 to 40 months apart.

### DETAILED DESCRIPTION

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to obscure aspects of the embodiments.

The present disclosure provides, among other aspects, systems and methods for identifying the disease status of a subject by evaluating changes in biological characteristics of the subject over time, as opposed to at a single time point as is done for convention disease detection assays. Specifically, by using data across multiple biological samples from a subject over time, personalized variance in biological characteristics of the subject can be accounted for when monitoring for a disease state.

For instance, conventional cancer diagnostics, whether using solid tumor samples or blood-based liquid biopsies, compare a subject's genomic aberrations attributable to cancerous tissue, identified from a single sample or a plurality of samples obtained at the same time, to genomic aberrations observed across a panel of controls. One limitation of this approach is that individuals may differ in their baseline level of aberration, making a generic cutoff on genomic anomalies restrictive. The theory underlying the systems and methods described herein can instead posit that each individual can be compared to a baseline state of themselves. This result can be improved sensitivity and specificity when detecting genomic aberrations, including novel genomic changes. This may be accomplished in a number of ways. For example, in one embodiment, intra-individual differences in a calculated probability of cancer are compared across time to intra-individual differences in a similarly-calculated probability of cancer in a panel of reference control subjects. In another embodiments, cancer probabilities determined from new samples from an individual are compared to cancer probabilities determined from previous samples from the individual, e.g., using a t-test which may or may not allow for incorporation of prior information from the panel of reference control subjects. In another embodiment, for more than two longitudinal samples, a trend test is performed on a series of calculated cancer probabilities, which may or may not be further compared to similar trend test results obtained for the panel of reference control subjects.

Advantageously, by accounting for some level of personal variance, the methods provided herein can increase the sensitivity and specificity of any underlying disease model, e.g., that provides a probability that the subject is afflicted with a particular disease state based on biological features measured from a single sample. For example, as described in Example 2, in silico experiments in which time series data for the progression of cancer was simulated using regression analysis demonstrates that the comparative methods described herein have the potential of increasing the sensitivity of stage 0 cancer detection by at least 100%, the sensitivity of stage I cancer detection by at least 70%, and the sensitivity of stage II cancer detection by at least 40%

### Definitions

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. The term “about” or “approximately” can mean within an order of magnitude, within 5-fold, or within 2-fold, of a value. Where particular values are described in the application and claims, unless otherwise stated the term “about” meaning within an acceptable error range for the particular value can be assumed. The term “about” can have the meaning as commonly understood by one of ordinary skill in the art. The term “about” can refer to ±10%. The term “about” can refer to ±5%.

As used herein, the term “genotypic” refers to a characteristic of the genome of an organism. Non-limiting examples of genotypic characteristics include those relating to the primary nucleic acid sequence of all or a portion of the genome (e.g., the presence or absence of a nucleotide polymorphism, indel, sequence rearrangement, mutational frequency, etc.), the copy number of one or more particular nucleotide sequences within the genome (e.g., copy number, allele frequency fractions, single chromosome or entire genome ploidy, etc.), the epigenetic status of all or a portion of the genome (e.g., covalent nucleic acid modifications such as methylation, histone modifications, nucleosome positioning, etc.), the expression profile of the organism's genome (e.g., gene expression levels, isotype expression levels, gene expression ratios, etc.). Accordingly, a “genotypic data construct” refers to a data construct, e.g., an electronic data file, that includes values for one or more genotypic characteristics of a subject. In some embodiments, a genotypic data construct includes one or more genotypic characteristics determined from a biological sample collected at a single time. In other embodiments, a genotypic data construct includes one or more genotypic characteristics determined from biological samples collected at several time points.

As used herein, the term “biological sample,” “patient sample,” or “sample” refers to any sample taken from a subject, which can reflect a biological state associated with the subject, and that includes cell free DNA. Examples of biological samples include, but are not limited to, blood, whole blood, plasma, serum, urine, cerebrospinal fluid, fecal, saliva, sweat, tears, pleural fluid, pericardial fluid, or peritoneal fluid of the subject. A biological sample can include any tissue or material derived from a living or dead subject. A biological sample can be a cell-free sample. A biological sample can comprise a nucleic acid (e.g., DNA or RNA) or a fragment thereof. The term “nucleic acid” can refer to deoxyribonucleic acid (DNA), ribonucleic acid (RNA) or any hybrid or fragment thereof. The nucleic acid in the sample can be a cell-free nucleic acid. A sample can be a liquid sample or a solid sample (e.g., a cell or tissue sample). A biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. A biological sample can be a stool sample. In various embodiments, the majority of DNA in a biological sample that has been enriched for cell-free DNA (e.g., a plasma sample obtained via a centrifugation protocol) can be cell-free (e.g., greater than 50%, 60%, 70%, 80%, 90%, 95%, or 99% of the DNA can be cell-free). A biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis.

As used herein, the term “cancer” or “tumor” refers to an abnormal mass of tissue in which the growth of the mass surpasses and is not coordinated with the growth of normal tissue. A cancer or tumor can be defined as “benign” or “malignant” depending on the following characteristics: degree of cellular differentiation including morphology and functionality, rate of growth, local invasion and metastasis. A “benign” tumor can be well differentiated, have characteristically slower growth than a malignant tumor and remain localized to the site of origin. In addition, in some cases a benign tumor does not have the capacity to infiltrate, invade or metastasize to distant sites. A “malignant” tumor can be a poorly differentiated (anaplasia), have characteristically rapid growth accompanied by progressive infiltration, invasion, and destruction of the surrounding tissue. Furthermore, a malignant tumor can have the capacity to metastasize to distant sites.

As used herein, the term “cancer condition” refers to breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, and gastric cancer. A cancer condition can be a predetermined stage of a breast cancer, a predetermined stage of a lung cancer, a predetermined stage of a prostate cancer, a predetermined stage of a colorectal cancer, a predetermined stage of a renal cancer, a predetermined stage of a uterine cancer, a predetermined stage of a pancreatic cancer, a predetermined stage of a cancer of the esophagus, a predetermined stage of a lymphoma, a predetermined stage of a head/neck cancer, a predetermined stage of a ovarian cancer, a predetermined stage of a hepatobiliary cancer, a predetermined stage of a melanoma, a predetermined stage of a cervical cancer, a predetermined stage of a multiple myeloma, a predetermined stage of a leukemia, a predetermined stage of a thyroid cancer, a predetermined stage of a bladder cancer, or a predetermined stage of a gastric cancer. A cancer condition can also be a survival metric, which can be a predetermined likelihood of survival for a predetermined period of time.

As used herein, the term “Circulating Cell-free Genome Atlas” or “CCGA” is defined as an observational clinical study that prospectively collects blood and tissue from newly diagnosed cancer patients as well as blood from subjects who do not have a cancer diagnosis. The purpose of the study is to develop a pan-cancer classifier that distinguishes cancer from non-cancer and identifies tissue of origin. Example 1 provides further details of the CCGA study.

The term “classification” can refer to any number(s) or other characters(s) that are associated with a particular property of a sample. For example, a “+” symbol (or the word “positive”) can signify that a sample is classified as having deletions or amplifications. In another example, the term “classification” can refer to an amount of tumor tissue in the subject and/or sample, a size of the tumor in the subject and/or sample, a stage of the tumor in the subject, a tumor load in the subject and/or sample, and presence of tumor metastasis in the subject. The classification can be binary (e.g., positive or negative) or have more levels of classification (e.g., fall into some numeric range supported or outputted by the classifier). The terms “cutoff” and “threshold” can refer to predetermined numbers used in an operation. For example, a cutoff size can refer to a size above which fragments are excluded. A threshold value can be a value above or below which a particular classification applies. Either of these terms can be used in either of these contexts.

As used herein, the terms “nucleic acid” and “nucleic acid molecule” are used interchangeably. The terms refer to nucleic acids of any composition form, such as deoxyribonucleic acid (DNA, e.g., complementary DNA (cDNA), genomic DNA (gDNA) and the like), and/or DNA analogs (e.g., containing base analogs, sugar analogs and/or a non-native backbone and the like), all of which can be in single- or double-stranded form. Unless otherwise limited, a nucleic acid can comprise known analogs of natural nucleotides, some of which can function in a similar manner as naturally occurring nucleotides. A nucleic acid can be in any form useful for conducting processes herein (e.g., linear, circular, supercoiled, single-stranded, double-stranded and the like). A nucleic acid in some embodiments can be from a single chromosome or fragment thereof (e.g., a nucleic acid sample may be from one chromosome of a sample obtained from a diploid organism). In certain embodiments nucleic acids comprise nucleosomes, fragments or parts of nucleosomes or nucleosome-like structures. Nucleic acids can comprise protein (e.g., histones, DNA binding proteins, and the like). Nucleic acids analyzed by processes described herein can be substantially isolated and are not substantially associated with protein or other molecules. Nucleic acids can also include derivatives, variants and analogs of DNA synthesized, replicated or amplified from single-stranded (“sense” or “antisense,” “plus” strand or “minus” strand, “forward” reading frame or “reverse” reading frame) and double-stranded polynucleotides. Deoxyribonucleotides can include deoxyadenosine, deoxycytidine, deoxyguanosine and deoxythymidine. A nucleic acid may be prepared using a nucleic acid obtained from a subject as a template.

As used herein, the term “cell-free nucleic acids” refers to nucleic acid molecules that can be found outside cells, in bodily fluids such as blood, whole blood, plasma, serum, urine, cerebrospinal fluid, fecal, saliva, sweat, sweat, tears, pleural fluid, pericardial fluid, or peritoneal fluid of a subject. Cell-free nucleic acids originate from one or more healthy cells and/or from one or more cancer cells Cell-free nucleic acids are used interchangeably as circulating nucleic acids. Examples of the cell-free nucleic acids include but are not limited to RNA, mitochondrial DNA, or genomic DNA. As used herein, the terms “cell free nucleic acid,” “cell free DNA,” and “cfDNA” are used interchangeably.

As used herein, the terms “control,” “control sample,” “reference,” “reference sample,” “normal,” and “normal sample” describe a sample from a subject that does not have a particular condition, or is otherwise healthy. In an example, a method as disclosed herein can be performed on a subject having a tumor, where the reference sample is a sample taken from a healthy tissue of the subject. A reference sample can be obtained from the subject, or from a database. The reference can be, e.g., a reference genome that is used to map sequence reads obtained from sequencing a sample from the subject. A reference genome can refer to a haploid or diploid genome to which sequence reads from the biological sample can be aligned and compared. An example of control sample can be DNA of white blood cells obtained from the subject. For a haploid genome, there can be one nucleotide at each locus. For a diploid genome, heterozygous loci can be identified; each heterozygous locus can have two alleles, where either allele can allow a match for alignment to the locus.

As used herein, the phrase “healthy” refers to a subject possessing good health. A healthy subject can demonstrate an absence of any malignant or non-malignant disease. A “healthy individual” can have other diseases or conditions, unrelated to the condition being assayed, which can normally not be considered “healthy.”

As used here, the term “high-signal cancer” means cancers with greater than 50% 5-year cancer-specific mortality. Examples of high-signal cancer include anorectal, colorectal, esophageal, head & neck, hepatobiliary, lung, ovarian, and pancreatic cancers, as well as lymphoma and multiple myeloma. High-signal cancers can be more aggressive and typically have an above-average cell-free nucleic acid concentration in test samples obtained from a patient. In some embodiments, “high signal cancers” refer to cancers that do not fall within the group of low signal cancers (e.g., uterine cancer, thyroid cancer, prostate cancer, and hormone-receptor-positive stage I/II breast cancer).

As used herein, the term “stage of cancer” (where the term “cancer” is either cancer generally or an enumerated cancer type) refers to whether cancer (or the enumerated cancer type when indicated) exists (e.g., presence or absence), a level of a cancer, a size of tumor, presence or absence of metastasis, the total tumor burden of the body, and/or other measure of a severity of a cancer (e.g., recurrence of cancer). The stage of cancer can be a number or other indicia, such as symbols, alphabet letters, and colors. The stage can be zero. The stage of cancer can also include premalignant or precancerous conditions (states) associated with mutations or a number of mutations. The stage of cancer can be used in various ways. For example, screening can check if cancer is present in someone who is not known previously to have cancer. Assessment can investigate someone who has been diagnosed with cancer to monitor the progress of cancer over time, study the effectiveness of therapies or to determine the prognosis. In one embodiment, the prognosis can be expressed as the chance of a subject dying of cancer, or the chance of the cancer progressing after a specific duration or time, or the chance of cancer metastasizing. Detection can comprise ‘screening’ or can comprise checking if someone, with suggestive features of cancer (e.g., symptoms or other positive tests), has cancer. A “level of pathology” can refer to level of pathology associated with a pathogen, where the level can be as described above for cancer. When the cancer is associated with a pathogen, a level of cancer can be a type of a level of pathology.

As used herein, the term “reference genome” refers to any particular known, sequenced or characterized genome, whether partial or complete, of any organism or virus that may be used to reference identified sequences from a subject. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or virus, expressed in nucleic acid sequences. As used herein, a reference sequence or reference genome can be an assembled or partially assembled genomic sequence from an individual or multiple individuals. In some embodiments, a reference genome is an assembled or partially assembled genomic sequence from one or more human individuals. The reference genome can be viewed as a representative example of a species' set of genes. In some embodiments, a reference genome comprises sequences assigned to chromosomes. Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38).

As used herein, the terms “sequencing,” “sequence determination,” and the like as used herein refers generally to any and all biochemical processes that may be used to determine the order of biological macromolecules such as nucleic acids or proteins. For example, sequencing data can include all or a portion of the nucleotide bases in a nucleic acid molecule such as a DNA fragment.

As used herein, the term “sequence reads” or “reads” refers to nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads). In some embodiments, sequence reads (e.g., single-end or paired-end reads) can be generated from one or both strands of a targeted nucleic acid fragment. The length of the sequence read can be associated with the particular sequencing technology. High-throughput methods, for example, can provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp, 2000 bp, 5000 bp, 10,000 bp, or 50,000 bp or more. Nanopore sequencing, for example, can provide sequence reads that can vary in size from tens to hundreds to thousands of base pairs. Illumina parallel sequencing can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

As used herein the term “sequencing breadth” refers to what fraction of a particular reference genome (e.g., human reference genome) or part of the genome has been analyzed. The denominator of the fraction can be a repeat-masked genome, and thus 100% can correspond to all of the reference genome minus the masked parts. A repeat-masked genome can refer to a genome in which sequence repeats are masked (e.g., sequence reads align to unmasked portions of the genome). Any parts of a genome can be masked, and thus one can focus on any particular part of a reference genome. Broad sequencing can refer to sequencing and analyzing at least 0.1% of the genome.

As used herein, the term “sequencing depth,” is interchangeably used with the term “coverage” and refers to the number of times a genomic location is surveyed during a sequencing process. For example, it can be reflected by the number of times that a locus is covered by a consensus sequence read corresponding to a unique nucleic acid target molecule aligned to the locus; e.g., the sequencing depth is equal to the number of unique nucleic acid target molecules covering the locus. The genomic location can be as small as a nucleotide, or as large as a chromosome arm, or as large as an entire genome. Sequencing depth can be expressed as “Yx”, e.g., 50×, 100×, etc., where “Y” refers to the number of times a genomic location is covered with a sequence corresponding to a nucleic acid target; e.g., the number of times independent sequence information is obtained covering the particular genomic location. In some embodiments, the sequencing depth corresponds to the number of genomes that have been sequenced. Sequencing depth can also be applied to multiple loci, or the whole genome, in which case Y can refer to the mean or average number of times a loci or a haploid genome, or a whole genome, respectively, is independently sequenced. When a mean depth is quoted, the actual depth for different loci included in the dataset can span over a range of values. In some embodiments, deep sequencing can refer to at least 100× in sequencing depth at a locus. In some embodiments, a sequencing depth of 10,000× or higher can be adopted in order to identify rare mutations.

As used herein, the term “sensitivity” or “true positive rate” (TPR) refers to the number of true positives divided by the sum of the number of true positives and false negatives. Sensitivity can characterize the ability of an assay or method to correctly identify a proportion of the population that truly has a condition. For example, sensitivity can characterize the ability of a method to correctly identify the number of subjects within a population having cancer. In another example, sensitivity can characterize the ability of a method to correctly identify the one or more markers indicative of cancer.

As used herein, the term “specificity” or “true negative rate” (TNR) refers to the number of true negatives divided by the sum of the number of true negatives and false positives. Specificity can characterize the ability of an assay or method to correctly identify a proportion of the population that truly does not have a condition. For example, specificity can characterize the ability of a method to correctly identify the number of subjects within a population not having cancer. In another example, specificity characterizes the ability of a method to correctly identify one or more markers indicative of cancer.

As used herein, the term “true positive” (TP) refers to a subject having a condition. “True positive” can refer to a subject that has a tumor, a cancer, a precancerous condition (e.g., a precancerous lesion), a localized or a metastasized cancer, or a non-malignant disease. “True positive” can refer to a subject having a condition, and is identified as having the condition by an assay or method of the present disclosure.

As used herein, the term “true negative” (TN) refers to a subject that does not have a condition or does not have a detectable condition. True negative can refer to a subject that does not have a disease or a detectable disease, such as a tumor, a cancer, a precancerous condition (e.g., a precancerous lesion), a localized or a metastasized cancer, a non-malignant disease, or a subject that is otherwise healthy. True negative can refer to a subject that does not have a condition or does not have a detectable condition, or is identified as not having the condition by an assay or method of the present disclosure.

As used herein, the term “single nucleotide variant” or “SNV” refers to a substitution of one nucleotide at a position (e.g., site) of a nucleotide sequence, e.g., a sequence corresponding to a target nucleic acid molecule from an individual, to a nucleotide that is different from the nucleotide at the corresponding position in a reference genome. A substitution from a first nucleobase X to a second nucleobase Y may be denoted as “X>Y.” For example, a cytosine to thymine SNV may be denoted as “C>T.” In some embodiments, an SNV does not result in a change in amino acid expression (a synonymous variant). In some embodiments, an SNV results in a change in amino acid expression (a non-synonymous variant).

As used herein, the term “methylation” refers to a modification of deoxyribonucleic acid (DNA) where a hydrogen atom on the pyrimidine ring of a cytosine base is converted to a methyl group, forming 5-methylcytosine. Methylation can occur at dinucleotides of cytosine and guanine referred to herein as “CpG sites”. In other instances, methylation may occur at a cytosine not part of a CpG site or at another nucleotide that's not cytosine; however, these are rarer occurrences. In this present disclosure, methylation can be discussed in reference to CpG sites for the sake of clarity. Anomalous cfDNA methylation can be identified as hypermethylation or hypomethylation, both of which may be indicative of cancer status. As is well known in the art, DNA methylation anomalies (compared to healthy controls) can cause different effects, which may contribute to cancer.

Various challenges arise in the identification of anomalously methylated cfDNA fragments. First, determining a subject's cfDNA to be anomalously methylated can hold weight in comparison with a group of control subjects, such that if the control group is small in number, the determination can lose confidence with the small control group. Additionally, among a group of control subjects' methylation status can vary which can be difficult to account for when determining a subject's cfDNA to be anomalously methylated. On another note, methylation of a cytosine at a CpG site can causally influence methylation at a subsequent CpG site.

The principles described herein can be equally applicable for the detection of methylation in a non-CpG context, including non-cytosine methylation. Further, the methylation state vectors may contain elements that are generally vectors of sites where methylation has or has not occurred (even if those sites are not CpG sites specifically). With that substitution, the remainder of the processes described herein are the same, and consequently, the inventive concepts described herein are applicable to those other forms of methylation.

As used herein the term “methylation index” for each genomic site (e.g., a CpG site, a region of DNA where a cytosine nucleotide is followed by a guanine nucleotide in the linear sequence of bases along its 5′→3′ direction) can refer to the proportion of sequence reads showing methylation at the site over the total number of reads covering that site. The “methylation density” of a region can be the number of reads at sites within a region showing methylation divided by the total number of reads covering the sites in the region. The sites can have specific characteristics, (e.g., the sites can be CpG sites). The “CpG methylation density” of a region can be the number of reads showing CpG methylation divided by the total number of reads covering CpG sites in the region (e.g., a particular CpG site, CpG sites within a CpG island, or a larger region). For example, the methylation density for each 100-kb bin in the human genome can be determined from the total number of unconverted cytosines (which can correspond to methylated cytosine) at CpG sites as a proportion of all CpG sites covered by sequence reads mapped to the 100-kb region. In some embodiments, this analysis is performed for other bin sizes, e.g., 50-kb or 1-Mb, etc. In some embodiments, a region is an entire genome or a chromosome or part of a chromosome (e.g., a chromosomal arm). A methylation index of a CpG site can be the same as the methylation density for a region when the region includes that CpG site. The “proportion of methylated cytosines” can refer the number of cytosine sites, “C's,” that are shown to be methylated (for example unconverted after bisulfite conversion) over the total number of analyzed cytosine residues, e.g., including cytosines outside of the CpG context, in the region. The methylation index, methylation density and proportion of methylated cytosines are examples of “methylation levels.”

As used herein, the term “methylation profile” (also called methylation status) can include information related to DNA methylation for a region. Information related to DNA methylation can include a methylation index of a CpG site, a methylation density of CpG sites in a region, a distribution of CpG sites over a contiguous region, a pattern or level of methylation for each individual CpG site within a region that contains more than one CpG site, and non-CpG methylation. A methylation profile of a substantial part of the genome can be considered equivalent to the methylome. “DNA methylation” in mammalian genomes can refer to the addition of a methyl group to position 5 of the heterocyclic ring of cytosine (e.g., to produce 5-methylcytosine) among CpG dinucleotides. Methylation of cytosine can occur in cytosines in other sequence contexts, for example, 5′-CHG-3′ and 5′-CHH-3′, where H is adenine, cytosine or thymine. Cytosine methylation can also be in the form of 5-hydroxymethylcytosine. Methylation of DNA can include methylation of non-cytosine nucleotides, such as N6-methyladenine.

As used herein, the terms “size profile” and “size distribution” can relate to the sizes of DNA fragments in a biological sample. A size profile can be a histogram that provides a distribution of an amount of DNA fragments at a variety of sizes. Various statistical parameters (also referred to as size parameters or just parameter) can distinguish one size profile to another. One parameter can be the percentage of DNA fragment of a particular size or range of sizes relative to all DNA fragments or relative to DNA fragments of another size or range.

As used herein, the term “subject” refers to any living or non-living organism, including but not limited to a human (e.g., a male human, female human, fetus, pregnant female, child, or the like), a non-human animal, a plant, a bacterium, a fungus or a protist. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a male or female of any age (e.g., a man, a women or a child).

As used herein, the term “tissue” refers to a group of cells that function together as a functional unit. More than one type of cell can be found in a single tissue. Different types of tissue may include different types of cells (e.g., hepatocytes, alveolar cells or blood cells), but also can correspond to tissue from different organisms (mother vs. fetus) or to healthy cells vs. tumor cells. The term “tissue” can generally refer to any group of cells found in the human body (e.g., heart tissue, lung tissue, kidney tissue, nasopharyngeal tissue, oropharyngeal tissue). In some aspects, the term “tissue” or “tissue type” can be used to refer to a tissue from which a cell-free nucleic acid originates. In one example, viral nucleic acid fragments can be derived from blood tissue. In another example, viral nucleic acid fragments can be derived from tumor tissue.

The terminology used herein is for the purpose of describing particular cases and is not intended to be limiting. As used herein, the singular forms “a,” “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

Several aspects are described below with reference to example applications for illustration. Numerous specific details, relationships, and methods are set forth to provide a full understanding of the features described herein. The features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are used to implement a methodology in accordance with the features described herein.

Plural instances may be provided for components, operations or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

Although the terms first, second, etc. may be used herein to describe various elements, these elements should not be limited by these terms. These terms are used to distinguish one element from another. For example, a first subject could be termed a second subject, and, similarly, a second subject could be termed a first subject, without departing from the scope of the present disclosure. The first subject and the second subject are both subjects, but they are not the same subject.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting (the stated condition or event (” or “in response to detecting (the stated condition or event),” depending on the context.

### System Embodiments

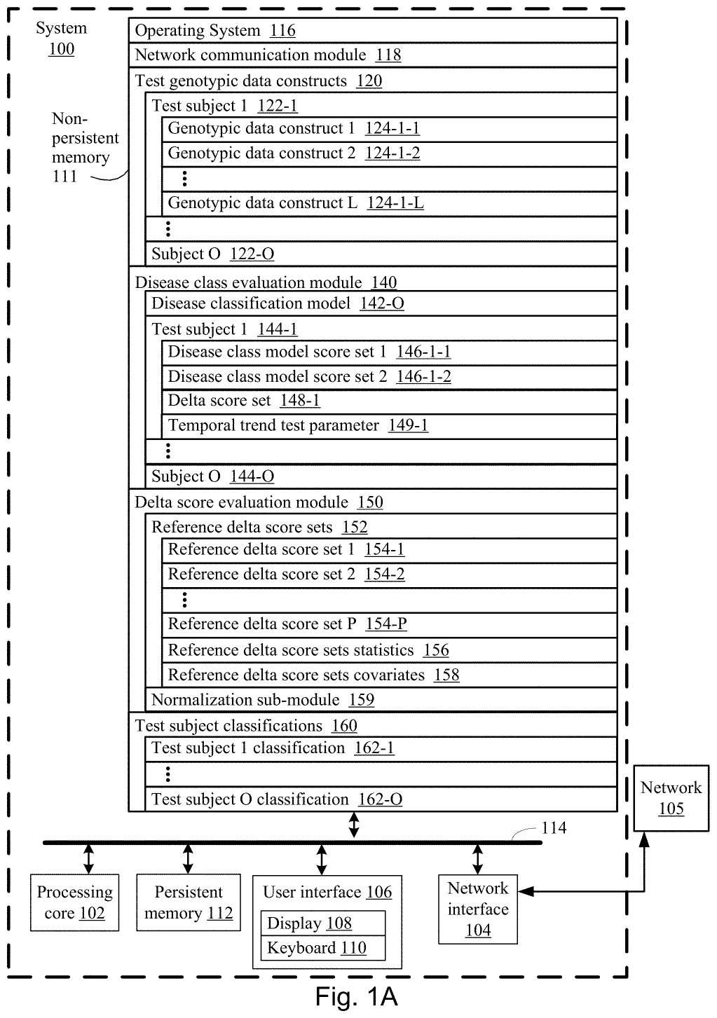

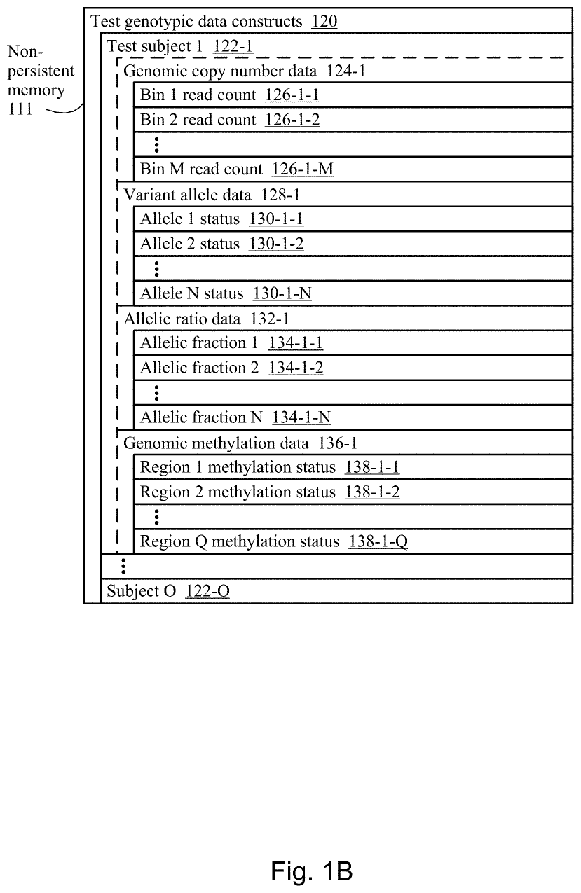

A detailed description of a system 100 for determining the disease state of a subject is described in conjunction with FIGS. 1A and 1B. As such, FIGS. 1A and 1B collectively illustrate the topology of a system, in accordance with an embodiment of the present disclosure.

Referring to FIG. 1A, in some embodiments, system 100 includes one or more computers. For purposes of illustration in FIG. 1A, system 100 is represented as a single computer that includes all of the functionality for identifying interactions within complex biological systems using data from a cell-based assay. However, in some embodiments, the functionality for determining the disease state of a subject is spread across any number of networked computers and/or resides on each of several networked computers and/or is hosted on one or more virtual machines at a remote location accessible across the communications network 105. Any of a wide array of different computer topologies can be used for the application and all such topologies are within the scope of the present disclosure.

Details of an exemplary system are now described in conjunction with FIG. 1. FIG. 1 is a block diagram illustrating a system 100 in accordance with some implementations. The device 100 in some implementations includes at least one or more processing units CPU(s) 102 (also referred to as processors), one or more network interfaces 104, a user interface 106, e.g., including a display 108 and/or keyboard 110, a memory 111, and one or more communication buses 114 for interconnecting these components. The one or more communication buses 114 optionally include circuitry (sometimes called a chipset) that interconnects and controls communications between system components. The memory 111 may be a non-persistent memory, a persistent memory, or any combination thereof. The non-persistent memory can include high-speed random access memory, such as DRAM, SRAM, DDR RAM, ROM, EEPROM, flash memory, whereas the persistent memory can include CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. Regardless of its specific implementation, the memory 111 comprises at least one non-transitory computer readable storage medium, and it stores thereon computer-executable executable instructions which can be in the form of programs, modules, and data structures.

In some embodiments, as shown in FIG. 1, the memory 111 stores:
instructions, programs, data, or information associated with an optional operating system 116, which includes procedures for handling various basic system services and for performing hardware-dependent tasks;
instructions, programs, data, or information associated with an optional network communication module (or instructions) 118 for connecting the system 100 with other devices and/or to a communication network 105;
a test genotypic data construct database 120 for storing sets 122 of genotypic data constructs 124 for test subjects, where each genotypic data construct 124 includes genotypic features acquired from sequencing cell-free DNA for the subject, e.g., one or more of genomic copy number data 124, e.g., bin read counts 126 for different regions of the genome of the subject, variant allele data 128, e.g., allele statuses 130 for different alleles within the genome of the subject, allelic ratio data 132, e.g., allele fractions 134 for different alleles within the genome of the subject, and genomic methylation data 136, e.g., CpG methylation statuses 138 for different genomic regions of the genome of the subject;
instructions, programs, data, or information associated with a disease class evaluation module 140 for interrogating one or more genotypic data constructs 124 for a test subject 122 using a disease classification model 142, to provide a disease class module score set 146 for a test subject 144; and
instructions, programs, data, or information associated with a delta score evaluation module 150 for evaluating a plurality of disease class model score sets 146 for a test subject against a reference delta score set 154, to provide a test subject classification 162, the delta score evaluation module 150 optionally applying one or more reference delta score set covariates 158 to either or both of a disease class model score set 146 and a reference delta score set 154 prior to evaluation and/or including a normalization sub-module to normalize either or both of a disease class model score set 146 and a reference delta score set 154 prior to evaluation.

In some implementations, modules 118, 140, and/or 150 and/or data stores 122, 144, 152, and/or 160 are accessible within any browser (e.g., installed on a phone, tablet, or laptop/desktop system). In some embodiments, modules 118, 140, and/or 150 run on native device frameworks, and are available for download onto the system 100 running an operating system 116, such as Windows, macOS, a Linux operating system, Android OS, or iOS.

In some implementations, one or more of the above identified data elements or modules of the system 100 for determining the disease state of a subject are stored in one or more of the previously described memory devices, and correspond to a set of instructions for performing a function described above. The above-identified data, modules or programs (e.g., sets of instructions) may not be implemented as separate software programs, procedures or modules, and thus various subsets of these modules may be combined or otherwise re-arranged in various implementations. In some implementations, the memory 111 optionally stores a subset of the modules and data structures identified above. Furthermore, in some embodiments the memory 111 stores additional modules and data structures not described above. In some embodiments, one or more of the above identified elements is stored in a computer system, other than that of system 100, that is addressable by system 100 so that system 100 may retrieve all or a portion of such data.

Although FIG. 1 depicts a “system 100,” the figure is intended as a functional description of the various features which may be present in computer systems than as a structural schematic of the implementations described herein. In practice, items shown separately can be combined and some items can be separated. Moreover, although FIG. 1 depicts certain data and modules in the memory 111 (which can be non-persistent or persistent memory), it can be appreciated that these data and modules, or portion(s) thereof, may be stored in more than one memory.

Any of the disclosed methods can make use of any of the assays or algorithms disclosed in U.S. Pat. No. 9,121,069 entitled “Diagnosing cancer using genomic sequencing;” US Pat. Pub. No. 2017/0218450A1 entitled “Detecting genetic aberrations associated with cancer using genomic sequencing;” U.S. Pat. No. 9,965,585 entitled “Detection of genetic or molecular aberrations associated with cancer;” U.S. Pat. No. 9,892,230 entitled “Size-based analysis of fetal or tumor DNA fraction in plasma,” US Pat. Pub. No. 2016/0201142A1 entitled “Using size and number aberrations in plasma DNA for detecting cancer;” US App. No. 62/642,461 entitled “Method and system for selecting, managing and analyzing data of high dimensionality;” U.S. App. No. 62/679,746 entitled “convolutional neural network systems and methods for data classification;” U.S. App. No. 62/777,693 entitled “Systems and Methods for Classifying Patients with Respect to Multiple Cancer Classes;” the disclosures of which are incorporated herein by reference, in their entireties, for all purposes. Accordingly, in some embodiments, system 100 disclosed herein may include any of the modules or data stores described in any of the above patents and patent applications.

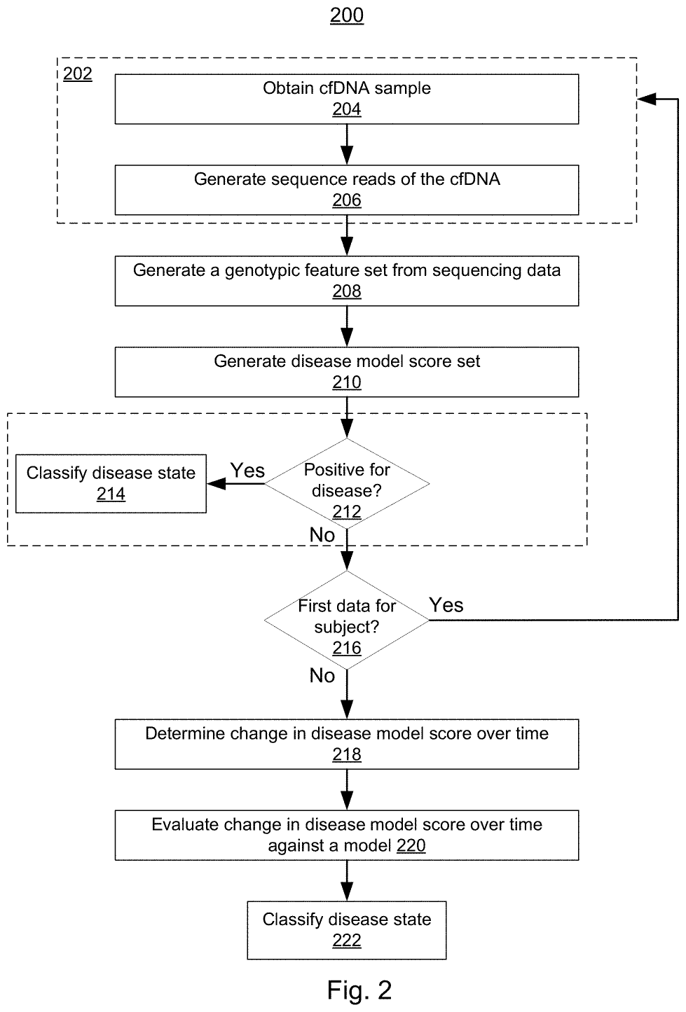

Now that details of a system 100 for determining the disease state of a subject have been disclosed, details regarding processes and features of the system, in accordance with various embodiment of the present disclosure, are disclosed below. Specifically, example processes are described below with reference to FIGS. 2, 3A-3G, and 4A-4F. In some embodiments, such processes and features of the system are carried out by modules 118, 140, and/or 150, as illustrated in FIG. 1. Referring to these methods, the systems described herein (e.g., system 100) can include instructions for performing the methods for determining the disease state of a subject.

FIG. 2 illustrates an example workflow 200 for determining the disease state of a subject, by evaluating changes in one or more biological signatures of the subject over time, in accordance with various embodiments of the present disclosure. Further details on various implementation of the steps illustrated in workflow 200 are described with more particularity below, e.g., in conjunction with the descriptions of examples methods 300 and 400. However, methods 300 and 400 can be example implementations of workflow 200, which can be suitable alternatives for performing each of the steps shown in workflow 200.

In some embodiments, the first step of workflow 200 is collection (202) of the underlying biological data from the subject at a first time. A biological sample can be collected (204) from the subject, e.g., at multiple time points. In some embodiments, as illustrated in FIG. 2, the biological sample used in the methods described herein includes cell-free nucleic acids, e.g., cfDNA. Advantageously, cell-free nucleic acids can be obtained by a minimally-invasive, small-volume blood draw from the subject, or possibly from non-invasive sampling of other bodily fluids such as saliva or urine. However, the systems and methods described herein can be suitable for evaluating any type of biological data that can be used to detect a disease state in a subject, e.g., cell-free or cellular genomic data, transcriptomic data, epigenetic data, proteomic data, metabolomic data, etc.

The biological samples can be processed to obtain biological information about the subject (206), e.g., one or more biological signatures for the subject at a given time point. In some embodiments, as illustrated in FIG. 2, cell-free nucleic acids (e.g., cfDNA) in the sample are sequenced to generate cfDNA sequence reads. For instance, many methods for next generation sequencing, which can be used for either DNA or RNA sequencing, can be used to isolate and sequence cell-free nucleic acid. These methods can include sequencing-by-synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), nanopore sequencing (Oxford Nanopore Technologies), or paired-end sequencing. However, as the methods described herein can be performed using other types of biological information, e.g., cell-free or cellular genomic data, transcriptomic data, epigenetic data, metabolomic data, etc., other methods for extracting biological features can also be contemplated herein, e.g., hybridization, qPCR, mass spectroscopy, immuno-affinity based detection methods, etc.

Although workflow 200 illustrates optional steps of collecting a biological sample (e.g., obtaining a cfDNA sample 204) and biological feature extraction (e.g., generating cfDNA sequence reads 206), in some embodiments the methods for determining the disease state of a subject described herein begin by obtaining previously extracted biological features (e.g., sequence reads), e.g., by receiving the biological features (e.g., sequence reads) in electronic form, e.g., over network 105.

Workflow 200 includes a step of generating (208) a biological feature set, based on the biological information collected at step 206. In some embodiments, as illustrated in FIG. 2, the biological feature set includes genotypic features (e.g., genotypic data constructs 122) acquired from sequence reads of a cell-free nucleic acid (e.g., cfDNA) sample. Examples of genotypic features useful for the methods described herein include read counts (e.g., bin read counts 126) which provide information about the relative abundance of particular sequences (e.g., genomic or exomic loci) in the test biological sample, the presence of variant alleles (e.g., allele statuses 130) which provide information about differences in the genome of the subject (e.g., in either or both of the germline or a diseased tissue) relative to a reference genome(s) for the species of the subject, allele frequencies (e.g., allele fractions 134) which provide information about the relative abundance of variant alleles, relative to non-variant alleles, in the test biological sample, and methylation statuses (e.g., CpG methylation statuses 138) which provide information about the methylation states of different genomic regions in the test biological sample. The particular features included in, and the formatting of, the data construct can be dictated by the classifier used in step 210 of workflow 200.

Accordingly, the biological feature set (e.g., a genotypic data construct 124) generated in step 208 can be applied (210) to a disease classifier (e.g., disease classification model 140) to generate a disease model score set (e.g., disease class model score set 146) for the subject at the first time. For instance, a probability that the subject has the disease condition (e.g., cancer, a particular type of cancer, a cardiovascular disease, etc.) at the time the biological sample was collected.

In some embodiments, as illustrated in workflow 200, the disease model score is used to initially classify (212) the subject as either having the disease state or not having the disease state (e.g., having cancer or not having cancer, having cardiovascular disease or not having cardiovascular disease, etc.). When the disease model score set indicates the disease state is present in the subject (e.g., the subject has cancer, the subject has cardiovascular disease, etc.), the subject can be classified (214) as having the disease condition, and evaluation of changes in a disease model score set for the subject over time are not used, because the subject has already been positively identified as having the disease state. However, when the disease model score set indicates the disease state is not present in the subject (e.g., the subject does not have cancer, the subject does not have cardiovascular disease, etc.), the methods described herein can be useful for identifying subjects who have the disease state, or are developing the disease state, but in which the disease state has not yet progressed sufficiently to enable identification via the disease classifier. For instance, cancer classifiers based on genotypic data acquired from cell-free DNA can use a minimal tumor fraction, in order to have enough signal to confidently identify a cancer signature. Advantageously, the methods described herein can be able to identify changes in biological data that indicate early disease states, even before the disease signal is strong enough for confident identification using conventional classifiers, e.g., that are based on data acquired at a single time point.

When the disease model score set (e.g., disease class model score set 146 generated at step 210) indicates the subject does not have the disease state, or indicates that the subject cannot be positively classified as having the disease state, the methods described herein can be used to compare changes in disease model score sets over time, to further interrogate whether the subject has a disease state that is not discernible by the single-time point classifier. However, the methods described herein can use biological data acquired from the subject at at least two different time points. Thus, when it is determined (216) that the disease model score set generated at step 210 of workflow 200 is the first such disease model score generated for the subject, biological data from another sample, acquired at a second time, can be used, as indicated by the arrow back to collection step 202 in FIG. 2.

In some embodiments, although a second disease model score set may not have been previously generated using the same classifier as used in step 210, biological data from the subject may be available from a different test, e.g., that was previously used in a different classifier. In some embodiments, there may be substantial overlap in the biological data collected for the two different assay to allow both data sets to be evaluated using a common classifier, e.g., either of the two classifiers previously used, or a third classifier that had not yet been employed. In this fashion, disease model scores can be generated for the subject at two different time points, allowing for a comparison to be performed, as described herein.

Accordingly, when one or more previously generated disease model score sets are available for the subject, e.g., generated using the same classifier, a different classifier with a known correspondence to the classifier used in step 210, or a classifier using biological data having substantial overlap with the biological data collected at step 202 to allow for generation of disease model scores for at least two time points, workflow 200 can proceed by determining a change (218) in the disease model score over time (e.g., delta score set 148 determined using disease class evaluation module 140). For instance, if a first disease model score set indicated a 12% chance of a disease state in the subject at a first time point and a second disease model score set indicated a 14% chance of a disease state in the subject at a second time point, a 2% change in the probability of the subject having the disease state occurred between the first and second time point. As described further below, in some embodiments, the change in disease model score over time is normalized or otherwise adjusted (e.g. as a covariate) for a parameter, such as the length of the period of time between the first and second time points, or a personal characteristic of the test subject (e.g., age, gender/biological sex, ethnicity, smoking status, familial history, etc.). The change in the disease model score over time determined in step 218 can be evaluated (220) against a model of change over time (e.g., using delta score evaluation module 150).

In some embodiments, as described further below in connection with method 300, the model includes a statistical test used to determine the probability of whether the change in the subject's disease model score over time (e.g., delta score set 148) belongs to a distribution of changes in disease model score over time determined from a population of reference subjects (e.g., reference delta score sets 152) that were classified as not having the disease state (or that could not be positively classified as having the disease state) using the same classifier as used in step 210 of workflow 200. In some embodiments, as described further below, this reference distribution is normalized against one or more parameters, such as the length of the period of time between the first and second time points, or a personal characteristic of the test subject (e.g., age, gender, ethnicity, smoking status, familial history, etc.), e.g., by application of one or more priors to the reference distribution, prior to evaluation of the test delta score set 148.

In other embodiments, as described further below in connection with method 400, when more than two delta score sets have been generated for the subject, that is the subject has been tested for the disease state at three or more points in time, the model includes application of a temporal trend test to all of the previous delta score sets 148 for the subject, to generate a test temporal trend test statistic, e.g., a measure of whether there is a statistically significant trend in the change of the delta score sets for the subject over time. The temporal trend test statistic for the subject can be compared, e.g., using a statistical hypothesis test, to a distribution of temporal trend test statistics (e.g., reference statistics 154) from a population of reference subjects that were classified as not having the disease state. In some embodiments, as described further below, this reference distribution is normalized against one or more parameter, such as a personal characteristic of the test subject (e.g., age, gender, ethnicity, smoking status, familial history, etc.), e.g., by application of one or more priors to the reference distribution, prior to evaluation of the test temporal trend test statistic.

Based on the comparison of the test value (e.g., the delta score set 148 or temporal trend test statistic), the disease state of the subject can be classified. For instance, in some embodiments, a statistical hypothesis test is performed with a null hypothesis that the subject's test value does not belong to the distribution of reference test values. When the null hypothesis is proved by the test, e.g., the test returns a statistically significant value satisfying a defined threshold (e.g., 0.05, 0.01, or 0.005), the subject can be classified as having the disease state. When the null hypothesis is not proved by the test, e.g., the test returns a statistically significant value that does not satisfy a defined threshold (e.g., 0.05, 0.01, or 0.005), the subject can be classified as not having the disease state.

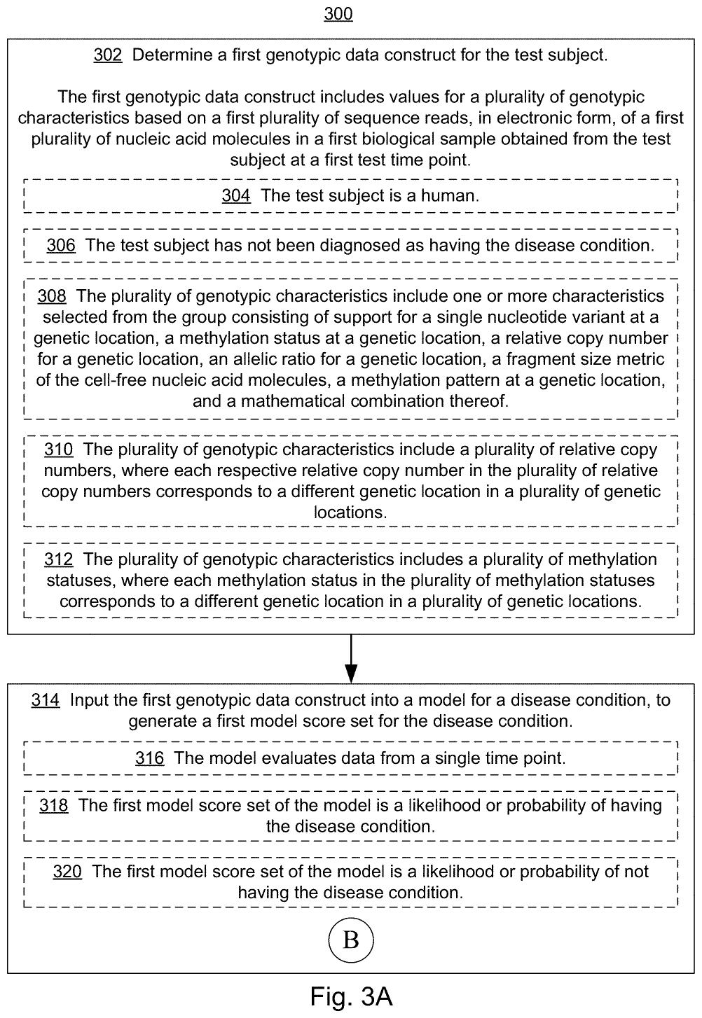

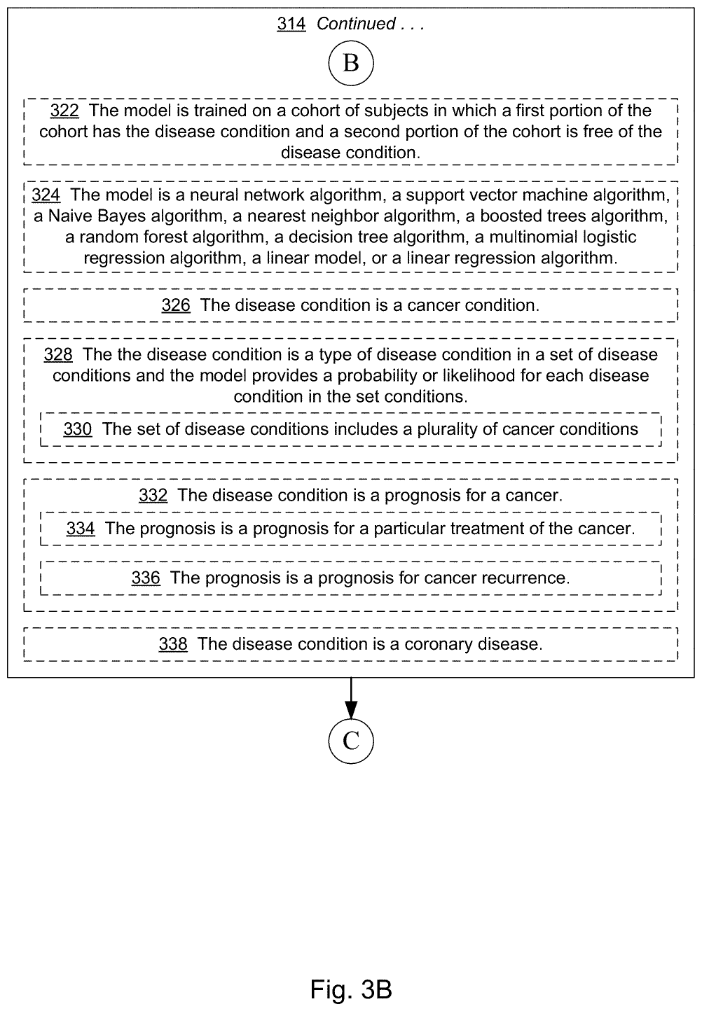

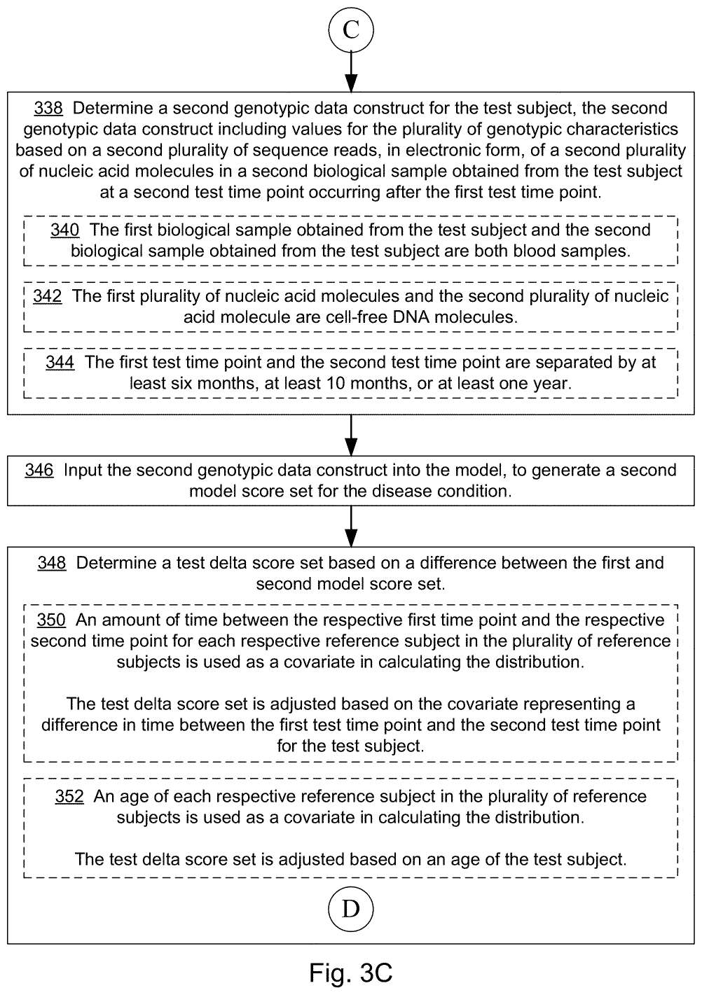

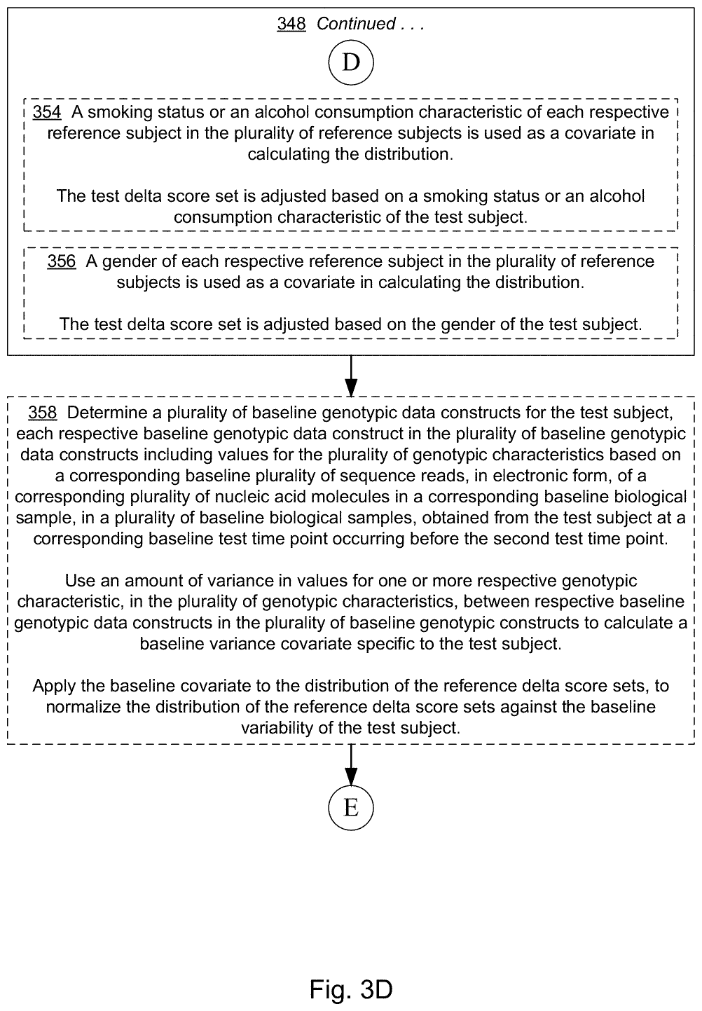

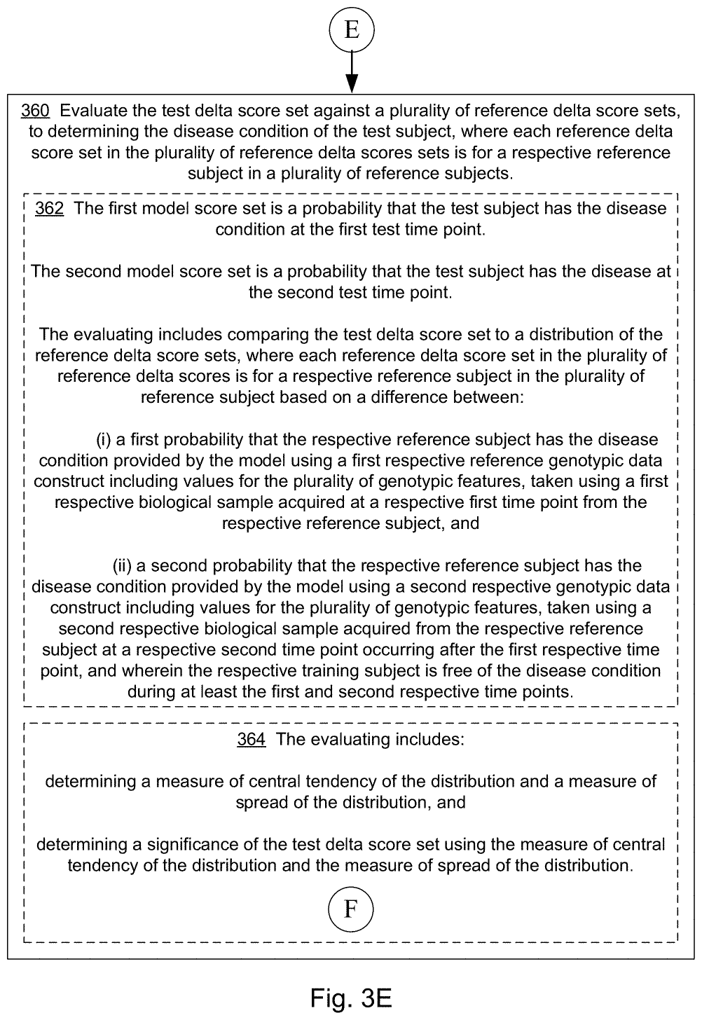

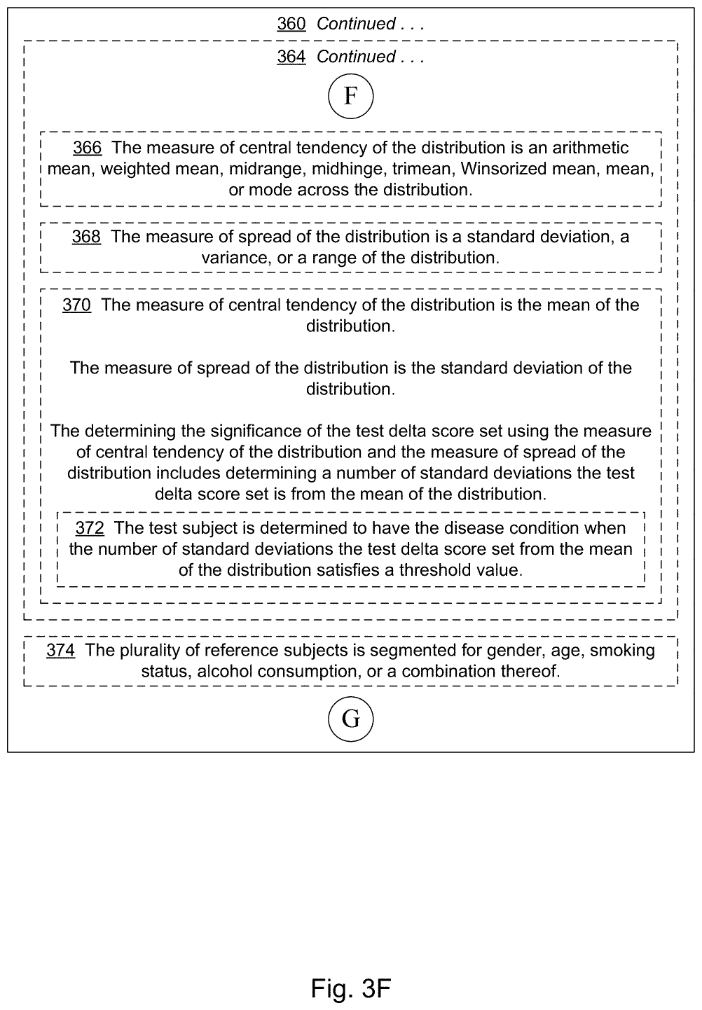

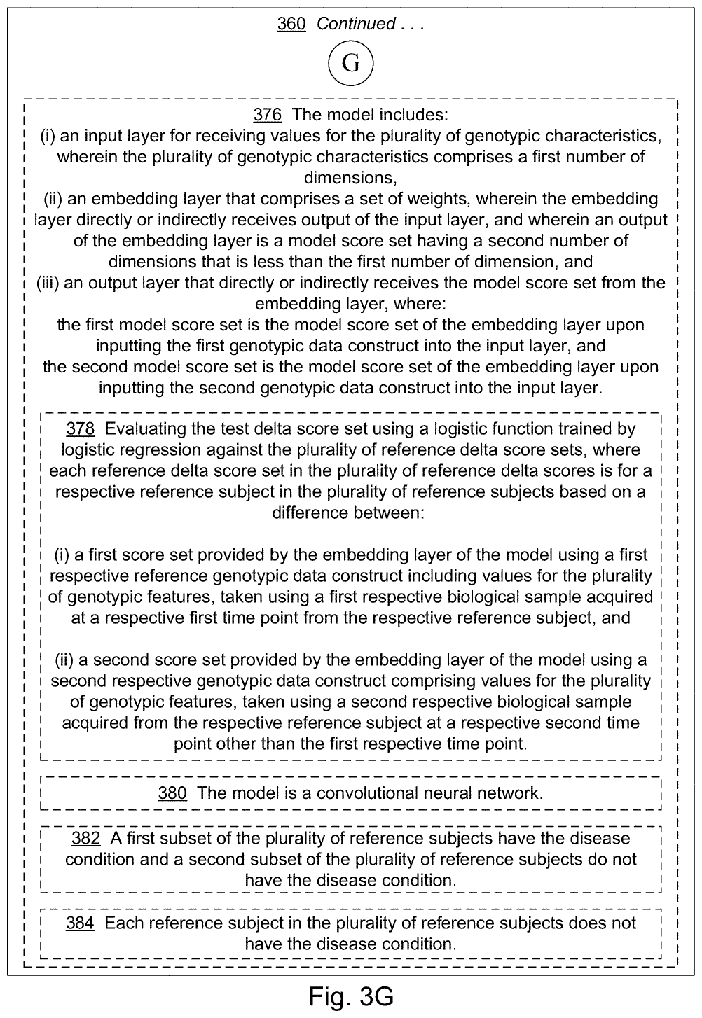

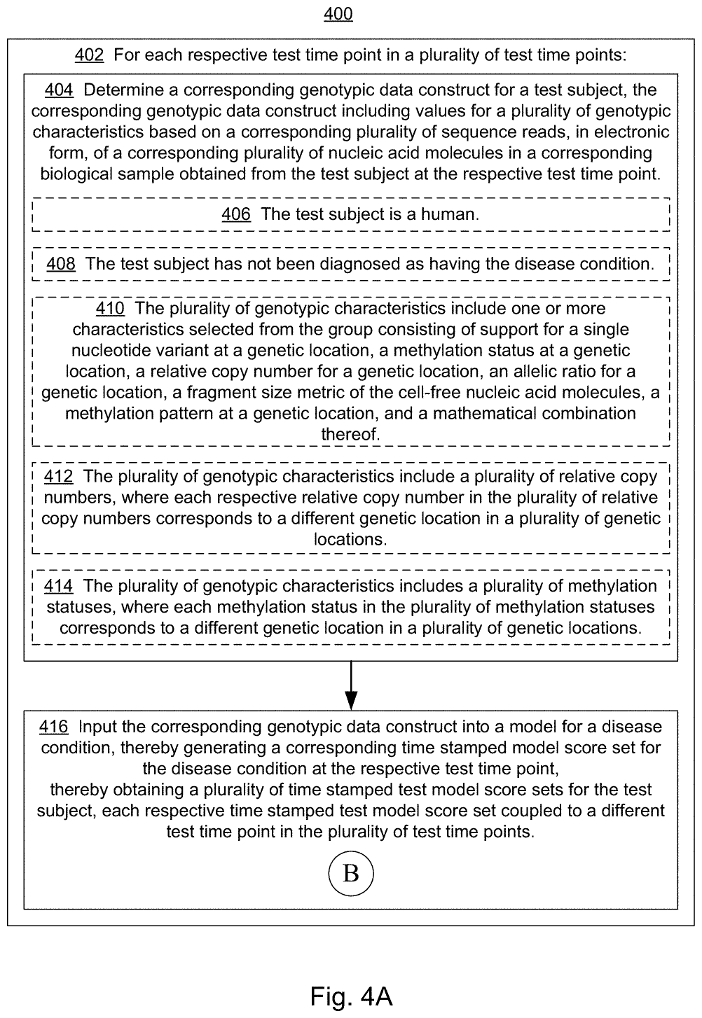

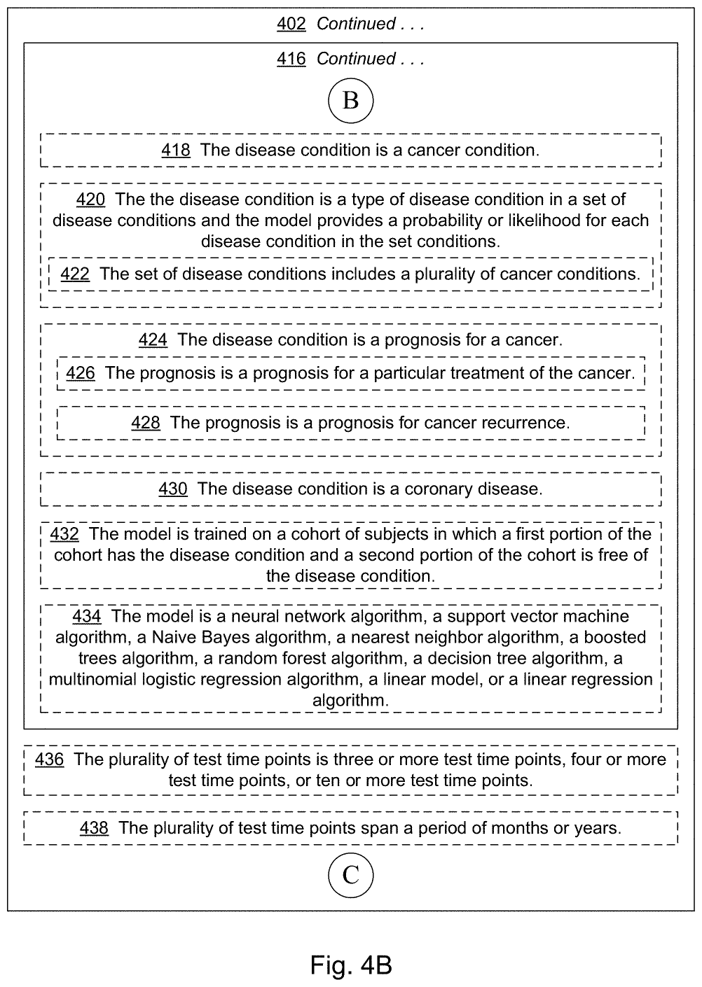

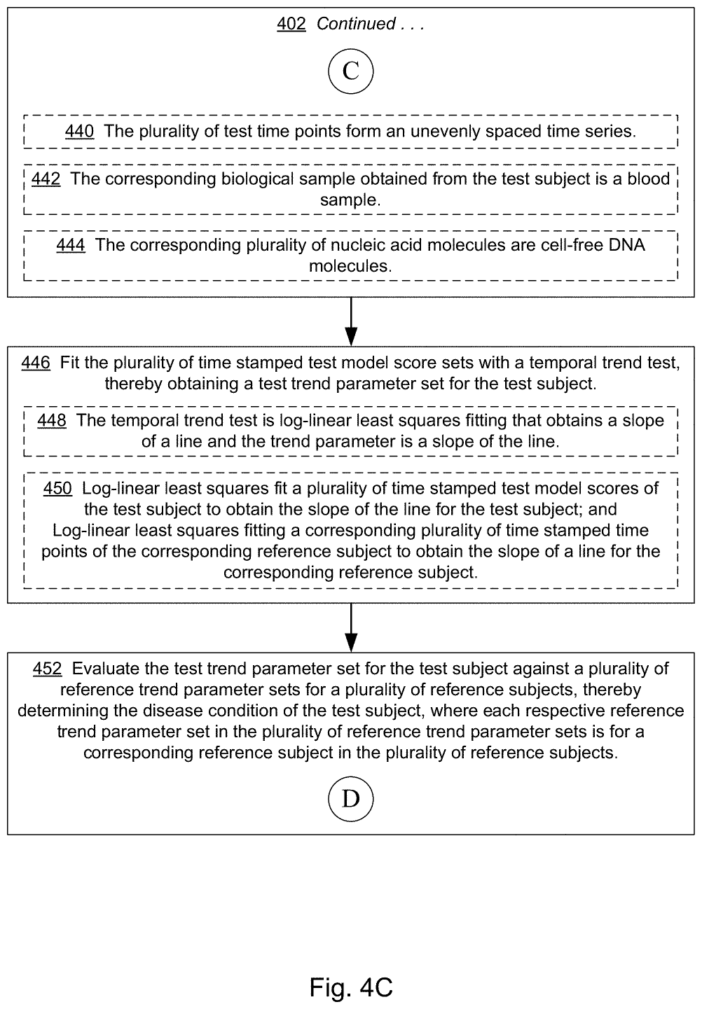

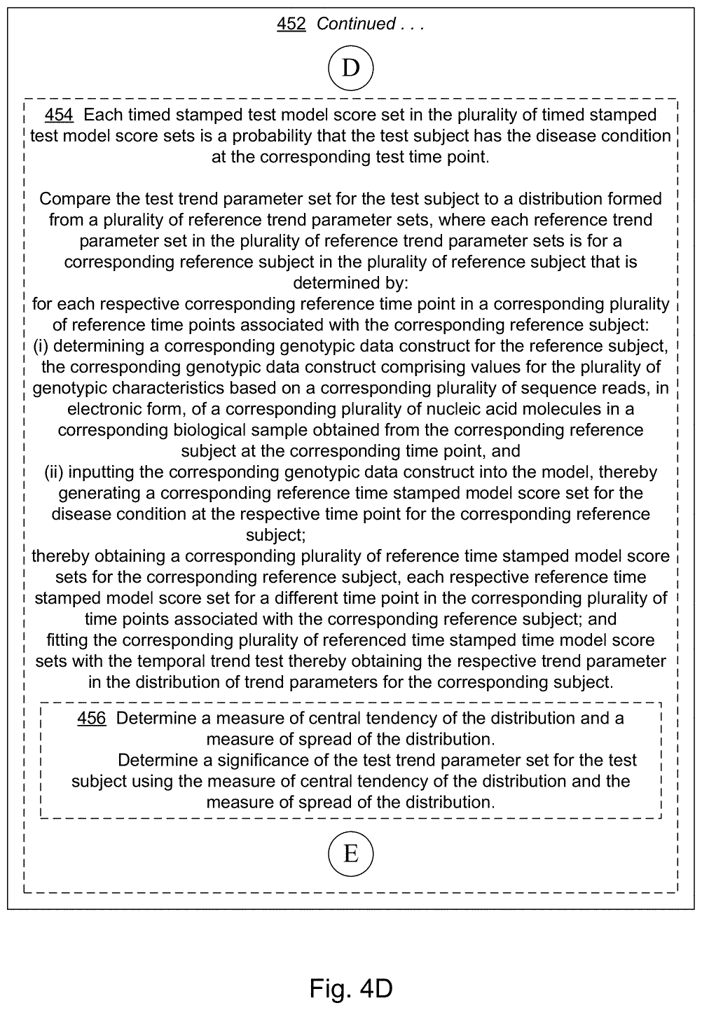

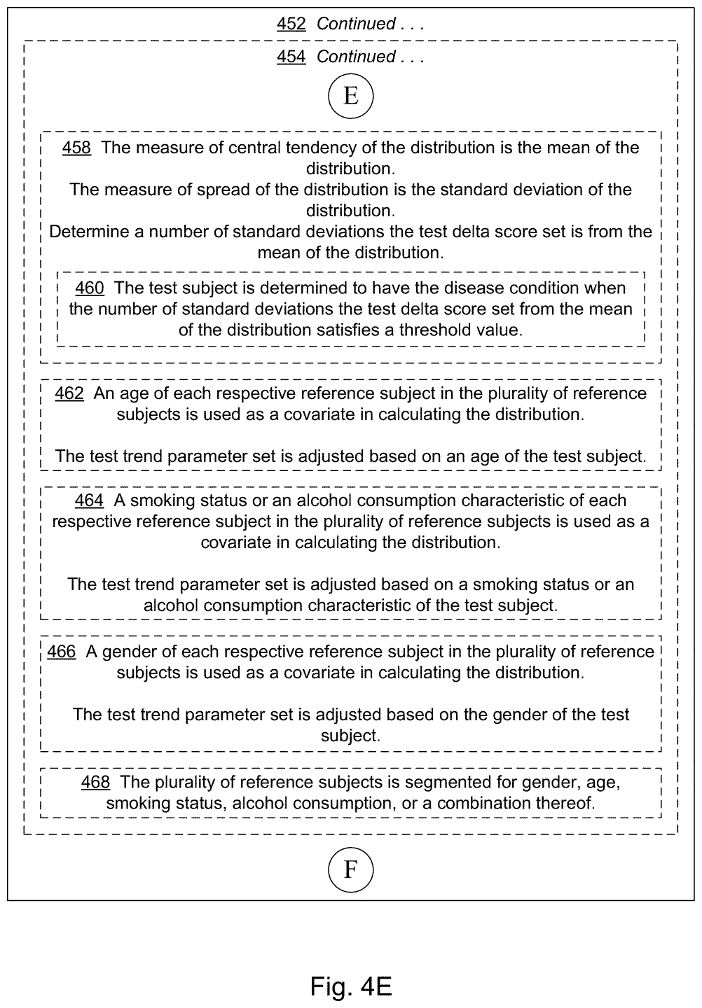

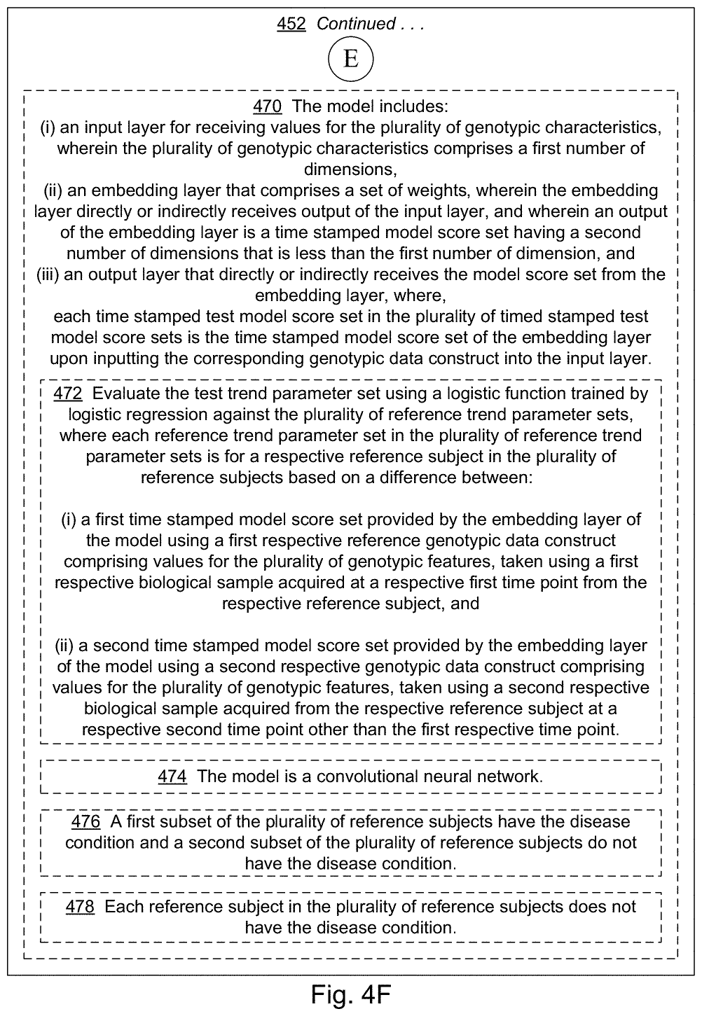

Having outlined a general workflow 200 for determining the disease state of a subject based on changes in biological characteristics of the subject over time, further description of the processes and features of the system, in accordance with various embodiments of the present disclosure, are disclosed below with reference to specific implementation methods 300 and 400, as illustrated in FIGS. 3A-3G and 4A-4F. In some embodiments, such processes and features of the system are carried out by modules 118, 140, and/or 150, as illustrated in FIG. 1. Referring to these methods, the systems described herein (e.g., system 100) can include instructions for performing the methods for determining the disease state of a subject. These particular processes and features for implementing the methods described herein are not intended to be limiting, and alternative processes and features can be used for performing individual steps of the disclosed methods.

Disease States

Generally, the systems and methods described herein can be used to increase the sensitivity and specificity of diagnosing any disease state that is associated with the development of a biological disease signature. That is, any disease state that can be diagnosed based on inspection of biological features of a subject, e.g., genomic features, epigenetic features, transcriptomic features, proteomic features, metabolomics features, and the like.

In some embodiments, the disease state is one that can be diagnosed based on genomic features of cell-free DNA (cfDNA). cfDNA is a particularly useful source of biological data for the methods described herein, because it is readily obtained from various body fluids, e.g., blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. Advantageously, use of bodily fluids can facilitate serial monitoring because of the ease of collection, as these fluids are collectable by non-invasive or minimally-invasive methodologies. This can be in contrast to methods that rely upon solid tissue samples, such as biopsies, which often times use invasive surgical procedures. Further, because bodily fluids such as blood circulate throughout the body, the cfDNA population can represents a sampling of many different tissue types from many different locations.

In some embodiments, the disease condition being tested for using the systems and methods described herein is a cancer condition (3026). For instance, methods for classifying various cancer conditions based on the evaluation of methylation patterns of cfDNA are described in U.S. Patent Application Publication No. 2019/0287652, the content of which is incorporated herein by reference for all purposes. Similarly, methods for classifying various cancer conditions based on the evaluation of relative genomic copy numbers in cfDNA are described in U.S. Patent Application Publication No. 2019/0287649, the content of which is incorporated herein by reference for all purposes. In some embodiments, the cancer can be an adrenal cancer, a biliary track cancer, a bladder cancer, a bone/bone marrow cancer, a brain cancer, a cervical cancer, a colorectal cancer, a cancer of the esophagus, a gastric cancer, a head/neck cancer, a hepatobiliary cancer, a kidney cancer, a liver cancer, a lung cancer, an ovarian cancer, a pancreatic cancer, a pelvis cancer, a pleura cancer, a prostate cancer, a renal cancer, a skin cancer, a stomach cancer, a testis cancer, a thymus cancer, a thyroid cancer, a uterine cancer, a lymphoma, a melanoma, a multiple myeloma, or a leukemia.

In some embodiments, the disease condition being tested for using the systems and methods described herein is a coronary disease (338). For instance, Zemmour H et al., Nat Commun., 9(1):1443 (2018), the content of which is incorporated herein by reference, identified genomic loci that are differentially non-methylated in cardiomyocytes and demonstrated that increases in these non-methylated sequences could be detected in the plasma of patients with acute ST-elevation myocardial infarction. Similarly, Khush K K et al., Am J Transplant., 19(10):2889-99 (2019), the content of which is incorporated herein by reference, demonstrated increases in donor-specific cfDNA following heart transplantation in samples classified as acute rejection. Similar results can be shown for kidney transplant rejections.

In some embodiments, the disease condition is a type of disease condition in a set of disease conditions and the model provides a probability or likelihood for each disease condition in the set conditions (3028). For instance, in some embodiments, the systems and methods described herein are able to detect and/or discriminate between several related diseases. For instance, diseases that present with similar symptoms and/or similar biological signatures. Similarly, in some embodiments, the systems and methods described herein are able to detect and/or discriminate between several different stages of one or more disease. For instance, between an early stage of a disease, a middle stage of a disease, and/or a late stage of a disease. An example are the various cancer stages, e.g., stages 0-IV.

In some embodiments, the set of disease conditions includes a plurality of cancer conditions (330). In some embodiments, the plurality of cancer conditions includes an adrenal cancer, a biliary track cancer, a bladder cancer, a bone/bone marrow cancer, a brain cancer, a cervical cancer, a colorectal cancer, a cancer of the esophagus, a gastric cancer, a head/neck cancer, a hepatobiliary cancer, a kidney cancer, a liver cancer, a lung cancer, an ovarian cancer, a pancreatic cancer, a pelvis cancer, a pleura cancer, a prostate cancer, a renal cancer, a skin cancer, a stomach cancer, a testis cancer, a thymus cancer, a thyroid cancer, a uterine cancer, a lymphoma, a melanoma, a multiple myeloma, or a leukemia.

Similarly, in some embodiments, the plurality of cancer conditions includes a predetermined stage of an adrenal cancer, a biliary track cancer, a bladder cancer, a bone/bone marrow cancer, a brain cancer, a cervical cancer, a colorectal cancer, a cancer of the esophagus, a gastric cancer, a head/neck cancer, a hepatobiliary cancer, a kidney cancer, a liver cancer, a lung cancer, an ovarian cancer, a pancreatic cancer, a pelvis cancer, a pleura cancer, a prostate cancer, a renal cancer, a skin cancer, a stomach cancer, a testis cancer, a thymus cancer, a thyroid cancer, a uterine cancer, a lymphoma, a melanoma, a multiple myeloma, or a leukemia.

In some embodiments, the disease condition is a prognosis for a disease. For example, a life expectancy without treatment, a life expectancy with treatment, or an expected response to a particular therapy. In some embodiments, the prognosis is a survival statistic, e.g., a disease-specific survival statistic (e.g., 1-year, 2-year, 5-year, 10-year, 20-year, or other survival time), a relative survival statistic (e.g., 1-year, 2-year, 5-year, 10-year, 20-year, or other survival time), an overall survival statistic (e.g., 1-year, 2-year, 5-year, 10-year, 20-year, or other survival time), or a disease-free survival statistic (e.g., 1-year, 2-year, 5-year, 10-year, 20-year, or other recurrence-free or progression-free survival time). In some embodiments, the prognosis is a predicted response to a particular therapeutic regimen. In some embodiments, the disease condition is a prognosis for a cancer (332). Accordingly, in some embodiments, the prognosis for the cancer is a prognosis for a particular treatment of the cancer (334). Similarly, in some embodiments, the prognosis for the cancer is a prognosis for cancer recurrence (336). In some embodiments, the disease condition is a prognosis for a coronary disease. In some embodiments, the disease condition is a prognosis for a particular treatment of a coronary disease.

Biological Sample Collection

As described herein, cfDNA can be a particularly useful source of biological data for the methods described herein, because it is readily obtained from various body fluids. Advantageously, use of bodily fluids can facilitate serial monitoring because of the ease of collection, as these fluids are collectable by non-invasive or minimally-invasive methodologies. This can be in contrast to methods that rely upon solid tissue samples, such as biopsies, which often times use invasive surgical procedures. Further, because bodily fluids, such as blood, circulate throughout the body, the cfDNA population can represent a sampling of many different tissue types from many different locations. Accordingly, in some embodiments, the biological samples obtained from the subject is selected from blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc.

In some embodiments, where the method includes evaluation of biological features (e.g., cfDNA) from two biological samples (e.g., as described below with reference to method 300), the first biological sample obtained from the test subject and the second biological sample obtained from the test subject independently include blood, whole blood, plasma, serum, urine, cerebrospinal fluid, fecal material, saliva, sweat, tears, pleural fluid, pericardial fluid, or peritoneal fluid of the subject. Similarly, in some embodiments where the method includes evaluation of biological features (e.g., cfDNA) from a series of more than two biological samples (e.g., as described below with reference to method 400), each of the samples obtained from the test subject independently include blood, whole blood, plasma, serum, urine, cerebrospinal fluid, fecal material, saliva, sweat, tears, pleural fluid, pericardial fluid, or peritoneal fluid of the subject.

In some embodiments, each sample in a series of samples from a test subject is of the same type. For instance, in some embodiments, where the method includes evaluation of biological features (e.g., cfDNA) from two biological samples (e.g., as described below with reference to method 300), the first biological sample obtained from the test subject and the second biological sample obtained from the test subject are the same type of sample, selected from blood, whole blood, plasma, serum, urine, cerebrospinal fluid, fecal material, saliva, sweat, tears, pleural fluid, pericardial fluid, and peritoneal fluid of the subject. In some embodiments, the first biological sample obtained from the test subject and the second biological sample obtained from the test subject are both blood samples. In some embodiments, the first biological sample obtained from the test subject and the second biological sample obtained from the test subject are both blood plasma samples.

Similarly, in some embodiments where the method includes evaluation of biological features (e.g., cfDNA) from a series of more than two biological samples (e.g., as described below with reference to method 400), each of the samples obtained from the test subject are the same type of sample, selected from blood, whole blood, plasma, serum, urine, cerebrospinal fluid, fecal material, saliva, sweat, tears, pleural fluid, pericardial fluid, and peritoneal fluid of the subject. In some embodiments, each of the biological samples obtained from the test subject in a series of biological samples are blood samples. In some embodiments, each of the biological samples obtained from the test subject in a series of biological samples are blood plasma samples.

Obtaining Biological Characteristics

As outlined above with reference to step 202 of workflow 200, in some embodiments, the methods described herein (e.g., method 300 and/or method 400) include a step of obtaining biological characteristics from a biological sample obtained from the test subject. For instance, in some embodiments the biological characteristics used by method 300 are sequence reads of cell-free DNA from a liquid sample from the subject. Accordingly, in some embodiments, the method includes one or both of obtaining a cfDNA sample from the subject and generating sequence reads from the cfDNA sample.

In some embodiments, e.g., as illustrated at step 206 of workflow 200, the biological features used in conjunction with the systems and methods described herein are genomic features acquired from a liquid biological sample from a subject. Advantageously, cell-free nucleic acids can be obtained by a minimally-invasive, small-volume blood draw from the subject, or possibly from non-invasive sampling of other bodily fluids such as saliva or urine. As described further below biological features (e.g., one or more of read counts 126, allele statuses 130, allelic fractions 134, and methylation statuses 138) can be extracted from sequence reads of the cell-free DNA present in liquid biological samples.

Accordingly, in some embodiments, the biological samples used in conjunction with the methods described herein (e.g., methods 300 and 400) are liquid samples containing any subset of the human genome, including the whole genome. The sample may be extracted from a subject known to have or suspected of having cancer. The sample may include blood, plasma, serum, urine, fecal, saliva, other types of bodily fluids, or any combination thereof. In some embodiments, methods for drawing a blood sample (e.g., syringe or finger prick) may be less invasive than procedures for obtaining a tissue biopsy, which may use surgery. The extracted sample may include cfDNA and/or ctDNA. In some embodiments, the sample is enriched for particular regions and/or loci of the genome, e.g., using probe-based enrichment methods.

A sequencing library can then be prepared from the sample, e.g., which may or may not have been enriched for particular sequences. In some embodiments, during library preparation, unique molecular identifiers (UMIs) are added to the nucleic acid molecules (e.g., DNA molecules) through adapter ligation. UMIs are short nucleic acid sequences (e.g., 4-10 base pairs) that are added to ends of DNA fragments during adapter ligation. In some embodiments, UMIs are degenerate base pairs that serve as a unique tag that can be used to identify sequence reads originating from a specific DNA fragment. In some embodiments, e.g., when multiplex sequencing can be used to sequence cfDNA from a plurality of subjects in a single sequencing reaction, a patient-specific index is also added to the nucleic acid molecules. In some embodiments, the patient specific index is a short nucleic acid sequence (e.g., 3-20 nucleotides) that are added to ends of DNA fragments during library construction, that serve as a unique tag that can be used to identify sequence reads originating from a specific patient sample. During PCR amplification following adapter ligation, the UMIs can be replicated along with the attached DNA fragment. This can provide a way to identify sequence reads that came from the same original fragment in downstream analysis.

In some embodiments, where the classification model evaluates the methylation status of one or more genomic locations, nucleic acids isolated from the biological sample (e.g., cfDNA) are treated to convert to convert unmethylated cytosines to uracils prior to generating the sequencing library. Accordingly, when the nucleic acids are sequenced, all cytosines called in the sequencing reaction can be methylated, since the unmethylated cytosines can be converted to uracils and accordingly would have been called as thymidines, rather than cytosines, in the sequencing reaction. Commercial kits can be available for bisulfite-mediated conversion of methylated cytosines to uracils, for instance, the EZ DNA Methylation™-Gold, EZ DNA Methylation™-Direct, and EZ DNA Methylation™-Lightning kit (available from Zymo Research Corp (Irvine, Calif.)). Commercial kits can also be available for enzymatic conversion of methylated cytosines to uracils, for example, the APOBEC-Seq kit (available from NEBiolabs, Ipswich, Mass.).

Sequence reads can then be generated from the sequencing library or pool of sequencing libraries. Sequencing data may be acquired by known means in the art. For example, next generation sequencing (NGS) techniques such as sequencing-by-synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), nanopore sequencing (Oxford Nanopore Technologies), or paired-end sequencing. In some embodiments, massively parallel sequencing is performed using sequencing-by-synthesis with reversible dye terminators.

In one embodiment, the sequencing is done using massively parallel sequencing. Massively parallel sequencing, such as that achievable on the 454 platform (Roche) (Margulies, M. et al. 2005 Nature 437, 376-380), Illumina Genome Analyzer (or Solexa platform) or SOLiD System (Applied Biosystems) or the Helicos True Single Molecule DNA sequencing technology (Harris T D et al. 2008 Science, 320, 106-109), the single molecule, real-time (SMRT™) technology of Pacific Biosciences, and nanopore sequencing (Soni G V and Meller A. 2007 Clin Chem 53: 1996-2001), allow the sequencing of many nucleic acid molecules isolated from a specimen at high orders of multiplexing in a parallel fashion (Dear Brief Funct Genomic Proteomic 2003; 1: 397-416). Each of these platforms sequences clonally expanded or even non-amplified single molecules of nucleic acid fragments.

As a high number of sequencing reads, in the order of hundreds of thousands to millions or even possibly hundreds of millions or billions, are generated from each sample in each run, the resultant sequenced reads form a representative profile of the mix of nucleic acid species in the original specimen. For example, the haplotype, transcriptome and methylation profiles of the sequenced reads resemble those of the original specimen (Brenner et al Nat Biotech 2000; 18: 630-634; Taylor et al Cancer Res 2007; 67: 8511-8518). Due to the large sampling of sequences from each specimen, the number of identical sequences, such as that generated from the sequencing of a nucleic acid pool at several folds of coverage or high redundancy, is also a good quantitative representation of the count of a particular nucleic acid species or locus in the original sample.

The sequence reads can then be aligned to a reference genome for the species of the subject using known methods in the art to determine alignment position information. Alignment position may generally describe a beginning position and an end position of a region in the reference genome that corresponds to a beginning nucleotide based and an end nucleotide base of a given sequence read.

In some embodiments, the biological characteristics used in the classifiers described herein include one or more of genomic data, epigenetic data, transcriptomic data, proteomic data, metabolomics data, and the like. In fact, the particular source and type of data may not be material to the methods described herein, so long as it can be used to discriminate between two or more disease states in a subject.

Method 300

In one aspect, the disclosure provides a method 300 that uses a population distribution to classify the disease state of a test subject based on changes in the probability or likelihood that the test subject has the disease state, as determined using a classifier trained to distinguish the disease state from one or more other disease states. Method 300 can relate directly to the disease states and methods for obtaining biological samples described above.

Referring generally to FIGS. 3A-3G, in some embodiments, the method includes determining a first genotypic data construct (e.g., genotypic data construct 124-1-1) for the test subject (e.g., as outlined above with reference to step 208 of workflow 200). The first genotypic data construct can include values for a plurality of genotypic characteristics (e.g., one or more of read counts 126, allele statuses 130, allelic fractions 134, and methylation statuses 138) based on a first plurality of sequence reads, in electronic form (e.g., cfDNA sequence reads generated at step 206 of workflow 200), of a first plurality of nucleic acid molecules in a first biological sample obtained from the test subject at a first test time point (e.g., a sample obtained at step 204 of workflow 200). The method can include inputting the first genotypic data construct into a model (e.g., disease classification model 142) for the disease condition (e.g., as outlined above with reference to step 210 of workflow 200), thereby generating a first model score set for the disease condition (e.g., disease class model score set 146-1-1). The method can include determining a second genotypic data construct (e.g., genotypic data construct 124-1-2) for the test subject (e.g., as outlined above with reference to repeating step 208 of workflow 200), the second genotypic data construct including values for the plurality of genotypic characteristics (e.g., the same one or more of read counts 126, allele statuses 130, allelic fractions 134, and methylation statuses 138 as included in first genotypic data construct 124-1-1) based on a second plurality of sequence reads, in electronic form (e.g., cfDNA sequence reads generated when step 206 of workflow 200 is repeated), of a second plurality of nucleic acid molecules in a second biological sample obtained from the test subject at a second test time point occurring after the first test time point (e.g., a sample obtained when step 204 of workflow 200 is repeated). The method can include inputting the second genotypic data construct into the model (e.g., the same disease classification model 142 as used for the first genotypic data construct), thereby generating a second model score set for the disease condition (e.g., disease class model score set 146-1-2). The method can include determining a test delta score set (e.g., delta score set 148-1) based on a difference between the first and second model score set (e.g., as outlined above with reference to step 218 of workflow 200). Then the method can include evaluating the test delta score set (e.g., as outlined above with reference to step 220 of workflow 200) against a plurality of reference delta score sets (e.g., reference delta score sets 152), thereby determining the disease condition of the test subject (e.g., test subject classification 162), where each reference delta score set (e.g., reference delta score sets 154) in the plurality of reference delta scores sets is for a respective reference subject in a plurality of reference subjects.

Generating First Biological Feature Sets

As outlined above with reference to step 208 of workflow 200, method 300 includes a step of generating a biological feature set (e.g., genotypic data construct 124) from the biological characteristics obtained from the biological sample. The particular features included in, and the formatting of, the biological feature set can be dictated by the classifier used (e.g., disease classification model 142) to determine an initial probability or likelihood that a particular disease state (e.g., cancer, a type of cancer, a cardiovascular disease, etc.). In some embodiments, the classifier uses genotypic features obtained from sequence reads acquired from a nucleic acid containing sample from the subject (e.g., a liquid sample containing cfDNA).

Accordingly, in some embodiments, the biological feature set includes features determined from a first plurality of nucleic acids in the first biological sample obtained from the subject. In some embodiments, the first plurality of nucleic acids include DNA molecules (e.g., cfDNA or genomic DNA). In some embodiments, the first plurality of nucleic acids include RNA molecules (e.g., mRNA). In some embodiments, the first plurality of nucleic acids include both DNA and RNA molecules.

Accordingly, in some embodiments, method 300 includes determining (302) a first genotypic data construct for the test subject. The first genotypic data construct includes values for a plurality of genotypic characteristics based on a first plurality of sequence reads (e.g., sequence reads obtained as described above with reference to step 206 illustrated in FIG. 2), in electronic form, of a first plurality of nucleic acid molecules in a first biological sample obtained from the test subject at a first test time point.

In some embodiments, the test subject is a human (304). In some embodiments, the test subject (e.g., a human) has not been diagnosed as having the disease condition (306). For instance, the methods described herein find utility in being able to identify a disease state in a subject before a biological signature for the disease reaches a level of detection (LOD) for a conventional classifier. Accordingly, in some embodiments, the subject has been tested for the disease state multiple times, and each time has been classified as not having the disease state.

In some embodiments (308), the genotypic characteristics include any characteristics including support for a single nucleotide variant at a genetic location (e.g., allele status 130), a methylation status at a genetic location (e.g., regional methylation status 138), a relative copy number for a genetic location (e.g., bin read count 126), an allelic ratio for a genetic location (e.g., allelic fraction 134), a fragment size metric of cell-free nucleic acid molecules, and a mathematical combination thereof.

Any methods for extracting genotypic features from a plurality of electronic sequence reads can be used. For instance, U.S. Patent Application Publication No. 2019/0287652, the content of which is incorporated herein by reference for all purposes, describes methods for determining the methylation status of a plurality of genomic locations. Similarly, U.S. Patent Application Publication No. 2019/0287649, the content of which is incorporated herein by reference for all purposes, describes methods for determining the relative copy number of a plurality of genomic locations. Likewise, methods for identifying single nucleotide variants and allele frequency of a plurality of genomic locations using next generation sequencing data is described, for instance, in Nielsen R. et al., PLoS One, 7(7):e37558 (2012), the content of which is incorporated herein by reference for all purposes.

In some embodiments, the plurality of genotypic characteristics include a plurality of relative copy numbers (e.g., bin read counts 126), where each respective relative copy number in the plurality of relative copy numbers corresponds to a different genetic location in a plurality of genetic locations (310). In some embodiments, the relative copy numbers represent the relative abundance of sequence reads from a plurality of genomic regions. In some embodiments, the genomic regions have the same size. In some embodiments, the genomic regions have different sizes.

In some embodiments, a genomic region is defined by the number of nucleic acid residues within the region. In some embodiments, a genomic region is defined by its location and the number of nucleic acids residues within the region. Any suitable size can be used to define genomic regions. For example, a genomic region can include 10 kb or fewer, 20 kb or fewer, 30 kb or fewer, 40 kb or fewer, 50 kb or fewer, 60 kb or fewer, 70 kb or fewer, 80 kb or fewer, 90 kb or fewer, 100 kb or fewer, 110 kb or fewer, 120 kb or fewer, 130 kb or fewer, 140 kb or fewer, 150 kb or fewer, 160 kb or fewer, 170 kb or fewer, 180 kb or fewer, 190 kb or fewer, 200 kb or fewer, or 250 kb or fewer.

In some embodiments, genomic regions are defined by dividing a reference genome for the species of the subject into a plurality of segments (i.e., the genomic regions). For instance, in certain embodiments, a reference genome is divided into up to 1,000 regions, 2,000 regions, 4,000 regions, 6,000 regions, 8,000 regions, 10,000 regions, 12,000 regions, 14,000 regions, 16,000 regions, 18,000 regions, 20,000 regions, 22,000 regions, 24,000 regions, 26,000 regions, 28,000 regions, 30,000 regions, 32,000 regions, 34,000 regions, 36,000 regions, 38,000 regions, 40,000 regions, 42,000 regions, 44,000 regions, 46,000 regions, 48,000 regions, 50,000 regions, 55,000 regions, 60,000 regions, 65,000 regions, 70,000 regions, 80,000 regions, 90,000 regions, or up to 100,000 regions. In some embodiments, sequence reads of a subject can be normalized to the average read count across all chromosomal regions for the subject, e.g., as described in U.S. Patent Application Publication No. 2019/0287649, the content of which is incorporated herein by reference, for all purposes.

In some embodiments, the copy number data is further normalized, e.g., to reduce or eliminate variance in the sequencing data caused by potential confounding factors. In some embodiments, the normalizing involves one or more of centering on a measure of central tendency within the sample, centering on data from a reference sample or cohort, normalization for GC content, and principal component analysis (PCA) correction. Additionally or alternatively, the normalization may include B-score processing, as described in U.S. Patent Application Publication No. 2019/0287649.

In some embodiments, the plurality of genotypic characteristics includes a plurality of methylation statuses (e.g., regional methylation statuses 138), where each methylation status in the plurality of methylation statuses corresponds to a different genetic location in a plurality of genetic locations (312). In some embodiments, each methylation status is represented by a methylation state vector as described, for example, in U.S. Provisional Patent Application No. 62/642,480, entitled “Methylation Fragment Anomaly Detection,” filed Mar. 13, 2018, which is hereby incorporated by reference herein in its entirety. In some embodiments, the methylation state vectors undergo p-value filtration and classification, as described in United States Patent Publication No. US 2019-0287652 A1, the content of which is incorporated herein by reference.

In some embodiments, the plurality of methylation statuses are obtained by a whole genome bisulfite sequencing (WGBS). In some embodiments, the plurality of methylation statuses is obtained by a targeted DNA methylation sequencing using a plurality of probes. In some embodiments, the plurality of probes hybridize to at least 100 loci in the human genome. In other embodiments, the plurality of probes hybridize to at least 250, 500, 750, 1000, 2500, 5000, 10,000, 25,000, 50,000, 100,000, or more loci in the human genome. Methods for identifying informative methylation loci for classifying a disease condition (e.g., cancer) are described, for instance, in U.S. Patent Application Publication No. 2019/0287649.

In some embodiments, the targeted DNA methylation sequencing detects one or more 5-methylcytosine (5 mC) and/or 5-hydroxymethylcytosine (5 hmC). In some embodiments, the targeted DNA methylation sequencing includes conversion of one or more unmethylated cytosines or one or more methylated cytosines to a corresponding one or more uracils. In some embodiments, the targeted DNA methylation sequencing includes conversion of one or more unmethylated cytosines to a corresponding one or more uracils, and the DNA methylation sequence reads out the one or more uracils as one or more corresponding thymines. In some embodiments, the targeted DNA methylation sequencing includes conversion of one or more methylated cytosines to a corresponding one or more uracils, and the DNA methylation sequence reads out the one or more 5 mC and/or 5 hmC as one or more corresponding thymines. In some embodiments, the conversion of one or more unmethylated cytosines or one or more methylated cytosines includes a chemical conversion, an enzymatic conversion, or combinations thereof.

Accordingly, in some embodiments, the plurality of genotypic characteristics for the first genotypic data structure (e.g., genotypic data construct 124-1-1) includes a first plurality of bin values (e.g., methylation statuses 138-1). Each respective bin value in the first plurality of bin values can represent a corresponding bin in a plurality of bins. Each respective bin value in the first plurality of bin values can be representative of a number of unique nucleic acid fragments with a predetermined methylation pattern identified using sequence reads in the first plurality of sequence reads that map to the corresponding bin in the plurality of bins. The plurality of genotypic characteristics for the second genotypic data structure (e.g., genotypic data construct 124-1-2) can include a second plurality of bin values (e.g., methylation statuses 138-1). Each respective bin value in the second plurality of bin values can represent a corresponding bin in the plurality of bins. Each respective bin value in the second plurality of bin values can be representative of a number of unique nucleic acid fragments with a predetermined methylation pattern identified using sequence reads in the second plurality of sequence reads that map to the corresponding bin in the plurality of bins. Each bin in the plurality of bins can represent a non-overlapping region of a reference genome of a species of the test subject.

In some embodiments, the methylation data is normalized, e.g., to reduce or eliminate variance in the sequencing data caused by potential confounding factors. In some embodiments, the normalizing involves one or more of centering on a measure of central tendency within the sample, centering on data from a reference sample or cohort, normalization for GC content, and principal component analysis (PCA) correction. Further description of normalization of methylation data can be found, for example, in U.S. Provisional Patent Application No. 62/642,480 and U.S. Patent Application Publication No. 2019/0287649.

In some embodiments, the methylation values are centered on a measure of central tendency within the sample. For example, in some embodiments, the normalizing includes determining a first measure of central tendency across the first plurality of bin values (e.g., methylation statuses 138-1 determined from a first biological sample from the subject obtained at a first time) and determining a second measure of central tendency across the second plurality of bin values (e.g., methylation statuses 138-2 determined from a second biological sample from the subject obtained at a second time). Then, each respective bin value in the first plurality of bin values (e.g., methylation statuses 138-1) can be replaced with the respective bin value divided by the first measure of central tendency and, similarly, each respective bin value in the second plurality of bin values (e.g., methylation statuses 138-1) with the respective bin value divided by the second measure of central tendency. In some embodiments, the first and second measures of central tendency are selected from an arithmetic mean, weighted mean, midrange, midhinge, trimean, Winsorized mean, mean, or mode across the corresponding plurality of bin values.

In some embodiments, the methylation values are normalized to correct for GC bias. For example, in some embodiments, the normalizing includes replacing each respective bin value in the first plurality of bin values (e.g., methylation statuses 138-1 determined from a first biological sample from the subject obtained at a first time) with the respective bin value corrected for a respective first GC bias in the first plurality of bin values, and replacing each respective bin value in the second plurality of bin values (e.g., methylation statuses 138-2 determined from a second biological sample from the subject obtained at a second time) with the respective bin value corrected for a respective second GC bias in the second plurality of bin values.

In some embodiments, the respective first GC bias is defined by a first equation for a curve or line fitted to a first plurality of two-dimensional points, where each respective two-dimensional point includes (i) a first value that is the respective GC content of the corresponding region of the reference genome represented by the respective bin in the first plurality of bins (e.g., methylation statuses 138-1) corresponding to the respective two-dimensional point and (ii) a second value that is the bin value in the first plurality of bin values for the respective bin. Then, the GC correction for the respective bin, derived from the GC content of the corresponding region of the reference genome of the species represented by the respective bin and the first equation, can be subtracted from the respective bin value. Similarly, the respective second GC bias can be defined by a second equation for a curve or line fitted to a first plurality of two-dimensional points, where each respective two-dimensional point includes (i) a third value that can be the respective GC content of the corresponding region of the reference genome represented by the respective bin in the second plurality of bins (e.g., methylation statuses 138-2) corresponding to the respective two-dimensional point and (ii) a fourth value that can be the bin value in the second plurality of bin values for the respective bin. Then, the GC correction for the respective bin, derived from the GC content of the corresponding region of the reference genome of the species represented by the respective bin and the second equation, can be subtracted from the respective bin value.

However, as described herein, in some embodiments, a particular classification model evaluates features other than genomic characteristics, e.g., instead of, or in addition to, the genomic characteristics described above. For instance, in some embodiments, the classification model evaluates epigenetic markers (epigenetics), gene expression profiling (transcriptomics), protein expression or activity profiling (proteomics), metabolic profiling (metabolomics), etc. Accordingly, in some embodiments, the biological feature sets formed include one or more of these non-genomic biological features.

Additionally, in some embodiments, the classification model evaluates one or more personal characteristics of the subject, e.g., gender, age, smoking status, alcohol consumption, familial history, etc., in addition to the biological features. Accordingly, in some embodiments, the biological feature sets formed includes one or more personal characteristics of the subject.

Generating a First Disease Model Score Set

As outlined above with reference to step 210 of workflow 200, method 300 includes using the first biological feature set formed from the biological characteristics obtained from the sample of the subject to generate a first disease model score set. Accordingly, in some embodiments, method 300 includes inputting (314) the first genotypic data construct into a model for the disease condition, thereby generating a first model score set for the disease condition. Generally, the identity and type of disease model used by the systems and methods described herein is immaterial.

Many different models that evaluate biological features in order to classifying one or more disease statuses (e.g., a cancer status, coronary disease status, etc.) of a subject have been developed. For instance, U.S. Patent Application Publication No. 2019/0287652 describes models that evaluate the methylation status across a plurality of genomic loci, e.g., using cfDNA samples, in order to classify a cancer status of a subject. Similarly, U.S. Patent Application Publication No. 2019/0287649 describes models that evaluate the relative copy number across a plurality of genomic loci, e.g., using cfDNA samples, in order to classify a cancer status of a subject. Likewise, various models have been developed that evaluate the presence of variant alleles (e.g., single nucleotide variants, indels, deletions, transversions, translocations, etc.) in order to classify a cancer status of a subject. Other suitable models are disclosed in U.S. patent application Ser. No. 16/428,575 entitled “Convolutional Neural Network Systems and Methods for Data Classification,” filed May 31, 2019. Generally, any model developed for the classification of a disease status of a subject may be used in conjunction with the systems and methods described herein.

In some embodiments, the model is for detecting the presence of a disease state in a subject, e.g., detecting cancer or coronary disease in a subject. That is, the systems and methods provided herein can be particularly well suited for improving upon the sensitivity and specificity of existing disease models, because they facilitate identity of changes in the biological signature of a subject over time, even when the biological signal is not yet strong enough for the underlying model to detect. Accordingly, in some embodiments, the model (e.g., the underlying model used to evaluate a genotypic data construct 124 at step 210 of workflow 200) evaluates data from a single time point (316). That can be samples that evaluate biological features acquired from a single sample from the subject, or from a plurality of samples acquired at a same or similar point in time from the subject (e.g., samples providing different types of biological information, such as genomic and transcriptomic information).

Generally, many different classification algorithms can find use in the systems and methods described herein. For instance, in some embodiments, the model is a neural network algorithm, a support vector machine algorithm, a Naive Bayes algorithm, a nearest neighbor algorithm, a boosted trees algorithm, a random forest algorithm, a decision tree algorithm, a multinomial logistic regression algorithm, a linear model, or a linear regression algorithm (324). Generally, the type of classifier used to generate a disease model score set for one or more disease states, using the systems and methods described herein, can be immaterial. In some embodiments, model is trained (322) on a cohort of subjects in which a first portion of the cohort has the disease condition and a second portion of the cohort is free of the disease condition, e.g., such that it is specifically trained to distinguish between a first state corresponding to not having the disease condition and a second state corresponding to having the disease condition.

Neural networks. In some embodiments, the classifier is a neural network or a convolutional neural network. Neural networks can be machine learning algorithms that may be trained to map an input data set to an output data set, where the neural network comprises an interconnected group of nodes organized into multiple layers of nodes. For example, the neural network architecture may comprise at least an input layer, one or more hidden layers, and an output layer. The neural network may comprise any total number of layers, and any number of hidden layers, where the hidden layers function as trainable feature extractors that allow mapping of a set of input data to an output value or set of output values. As used herein, a deep learning algorithm (DNN) can be a neural network comprising a plurality of hidden layers, e.g., two or more hidden layers. Each layer of the neural network can comprise a number of nodes (or “neurons”). A node can receive input that comes either directly from the input data or the output of nodes in previous layers, and perform a specific operation, e.g., a summation operation. In some embodiments, a connection from an input to a node is associated with a weight (or weighting factor). In some embodiments, the node may sum up the products of all pairs of inputs, xᵢ, and their associated weights. In some embodiments, the weighted sum is offset with a bias, b. In some embodiments, the output of a node or neuron may be gated using a threshold or activation function, f, which may be a linear or non-linear function. The activation function may be, for example, a rectified linear unit (ReLU) activation function, a Leaky ReLu activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training data set and a gradient descent or backward propagation method so that the output value(s) that the ANN computes are consistent with the examples included in the training data set. The parameters may be obtained from a back propagation neural network training process.

Any of a variety of neural networks may be suitable for use in analyzing product development. Examples can include, but are not limited to, feedforward neural networks, radial basis function networks, recurrent neural networks, convolutional neural networks, and the like. In some embodiments, the machine learning makes use of a pre-trained ANN or deep learning architecture. Convolutional neural networks can be used for classifying methylation patterns in accordance with the present disclosure.

Support vector machines. In some embodiments, the classifier is a support vector machine (SVM). When used for classification, SVMs separate a given set of binary labeled data with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space can correspond to a non-linear decision boundary in the input space.

Naïve Bayes algorithms. Naive Bayes classifiers can be a family of “probabilistic classifiers” based on applying Bayes' theorem with strong (naïve) independence assumptions between the features. In some embodiments, they are coupled with Kernel density estimation. In some embodiments, the classifier is a Naive Bayes algorithm.

Nearest neighbor algorithms. Nearest neighbor classifiers can be memory-based and include no classifier to be fit. Given a query point xo, the k training points x₍ᵣ₎, r, . . . , k closest in distance to xo can be identified and then the point xo is classified using the k nearest neighbors. Ties can be broken at random. In some embodiments, Euclidean distance in feature space is used to determine distance as:

d₍ᵢ₎=∥x₍ᵢ₎−x₍₀₎∥

In some embodiments, when the nearest neighbor algorithm is used, the bin values for the training set can be standardized to have mean zero and variance 1. In some embodiments, the nearest neighbor analysis is refined to address issues of unequal class priors, differential misclassification costs, and feature selection. Many of these refinements can involve some form of weighted voting for the neighbors. In some embodiments, the classifier is a nearest neighbor algorithm.

Random forest, decision tree, and boosted tree algorithms. In some embodiments, the classifier is a decision tree. Tree-based methods can partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests.

Regression. In some embodiment, a regression algorithm is used as the classifier. A regression algorithm can be any type of regression. For example, in some embodiments, the regression algorithm is logistic regression. In some embodiments, the regression algorithm is logistic regression with lasso, L2 or elastic net regularization. In some embodiments, those extracted features that have a corresponding regression coefficient that fails to satisfy a threshold value are pruned (removed from) consideration. In some embodiments, a generalization of the logistic regression model that handles multicategory responses is used as the classifier. In some embodiments, the classifier makes use of a regression model.

Linear discriminant analysis algorithms. Linear discriminant analysis (LDA), normal discriminant analysis (NDA), or discriminant function analysis can be a generalization of Fisher's linear discriminant, a method used in statistics, pattern recognition, and machine learning to find a linear combination of features that characterizes or separates two or more classes of objects or events. The resulting combination can be used as the classifier (linear classifier) in some embodiments of the present disclosure.

Mixture model. In some embodiments, the classifier is a mixture model. See, for example, United States Patent Publication No. US 2020-0365229 A1, which is hereby incorporated by reference.

Hidden Markov model. In some embodiments, in particular, those embodiments including a temporal component, the classifier is a hidden Markov model.

Gaussian process. In some embodiments, for classification, the logit transformed probability is modeled as a Gaussian process.

Penalized model. In some embodiments, temporal information is used for penalties when learning the weights for a model (e.g., a classifier). In this situation, the temporal trend in cancer probability can be smooth and penalties can be used to penalize for this smoothness.

Clustering. In some embodiments, the classifier is an unsupervised clustering model. In some embodiments, the classifier is a supervised clustering model. The clustering problem can be described as one of finding natural groupings in a dataset. To identify natural groupings, two issues can be addressed. First, a way to measure similarity (or dissimilarity) between two samples can be determined. This metric (e.g., similarity measure) can be used to ensure that the samples in one cluster are more like one another than they are to samples in other clusters. Second, a mechanism for partitioning the data into clusters using the similarity measure can be determined. One way to begin a clustering investigation can be to define a distance function and to compute the matrix of distances between all pairs of samples in the training set. If distance is a good measure of similarity, then the distance between reference entities in the same cluster can be significantly less than the distance between the reference entities in different clusters. However, clustering may not use of a distance metric. For example, a nonmetric similarity function s(x, x′) can be used to compare two vectors x and x′. s(x, x′) can be a symmetric function whose value is large when x and x′ are somehow “similar.” Once a method for measuring “similarity” or “dissimilarity” between points in a dataset has been selected, clustering can use a criterion function that measures the clustering quality of any partition of the data. Partitions of the data set that extremize the criterion function can be used to cluster the data. Particular exemplary clustering techniques that can be used in the present disclosure can include, but are not limited to, hierarchical clustering (agglomerative clustering using a nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering (e.g., with no preconceived number of clusters and/or no predetermination of cluster assignments).

The A score classifier described herein can be a classifier of tumor mutational burden based on targeted sequencing analysis of nonsynonymous mutations. For example, a classification score (e.g., “A score”) can be computed using logistic regression on tumor mutational burden data, where an estimate of tumor mutational burden for each individual is obtained from the targeted cfDNA assay. In some embodiments, a tumor mutational burden can be estimated as the total number of variants per individual that are: called as candidate variants in the cfDNA, passed noise-modeling and joint-calling, and/or found as nonsynonymous in any gene annotation overlapping the variants. The tumor mutational burden numbers of a training set can be fed into a penalized logistic regression classifier to determine cutoffs at which 95% specificity is achieved using cross-validation.

The B score classifier is described in U.S. Patent Publication No. 62/642,461, filed 62/642,461, which is hereby incorporated by reference. In accordance with the B score method, a first set of sequence reads of nucleic acid samples from healthy subjects in a reference group of healthy subjects can be analyzed for regions of low variability. Accordingly, each sequence read in the first set of sequence reads of nucleic acid samples from each healthy subject can be aligned to a region in the reference genome. From this, a training set of sequence reads from sequence reads of nucleic acid samples from subjects in a training group can be selected. Each sequence read in the training set can align to a region in the regions of low variability in the reference genome identified from the reference set. The training set can include sequence reads of nucleic acid samples from healthy subjects as well as sequence reads of nucleic acid samples from diseased subjects who are known to have the cancer. The nucleic acid samples from the training group can be of a type that is the same as or similar to that of the nucleic acid samples from the reference group of healthy subjects. From this it can be determined, using quantities derived from sequence reads of the training set, one or more parameters that reflect differences between sequence reads of nucleic acid samples from the healthy subjects and sequence reads of nucleic acid samples from the diseased subjects within the training group. Then, a test set of sequence reads associated with nucleic acid samples comprising cfNA fragments from a test subject whose status with respect to the cancer is unknown can be received, and the likelihood of the test subject having the cancer can be determined based on the one or more parameters.

The M score classifier is described in U.S. Patent Application No. 62/642,480, entitled “Methylation Fragment Anomaly Detection,” filed Mar. 13, 2018, which is hereby incorporated by reference.

Ensembles of classifiers and boosting. In some embodiments, an ensemble (two or more) of classifiers is used. In some embodiments, a boosting technique such as AdaBoost is used in conjunction with many other types of learning algorithms to improve the performance of the classifier. In this approach, the output of any of the classifiers disclosed herein, or their equivalents, can be combined into a weighted sum that represents the final output of the boosted classifier.

In some aspects, the disclosed methods can work in conjunction with cancer classification models. The cancer classification models can be any models described elsewhere herein. For example, a machine learning or deep learning model (e.g., a disease classifier) can be used to determine a disease state based on values of one or more features determined from one or more cell-free DNA molecules or sequence reads (e.g., derived from one or more cfDNA molecules). In various embodiments, the output of the machine learning or deep learning model is a predictive score or probability of a disease state (e.g., a predictive cancer score).

In some embodiments, the machine-learned model includes a logistic regression classifier. In other embodiments, the machine learning or deep learning model can be one of a decision tree, an ensemble (e.g., bagging, boosting, random forest), gradient boosting machine, linear regression, Naïve Bayes, or a neural network. The disease state model can include learned weights for the features that are adjusted during training. The term “weights” is used generically here to represent the learned quantity associated with any given feature of a model, regardless of which particular machine learning technique is used. In some embodiments, a cancer indicator score is determined by inputting values for features derived from one or more DNA sequences (or DNA sequence reads thereof) into a machine learning or deep learning model.

During training, training data can be processed to generate values for features that are used to train the weights of the disease state model. As an example, training data can include cfDNA data, cancer gDNA, and/or WBC gDNA data obtained from training samples, as well as an output label. For example, the output label can be an indication as to whether the individual is known to have a specific disease (e.g., known to have cancer) or known to be healthy (i.e., devoid of a disease). In other embodiments, the model can be used to determine a disease type, or tissue of origin (e.g., cancer tissue of origin), or an indication of a severity of the disease (e.g., cancer stage) and generate an output label therefor. Depending on the particular embodiment, the disease state model can receive the values for one or more of the features determine from a DNA assay used for detection and quantification of a cfDNA molecule or sequence derived therefrom, and computational analyses relevant to the model to be trained. In one embodiment, the one or more features comprise a quantity of one or more cfDNA molecules or sequence reads derived therefrom. Depending on the differences between the scores output by the model-in-training and the output labels of the training data, the weights of the predictive cancer model can be optimized to enable the disease state model to make more accurate predictions. In various embodiments, a disease state model may be a non-parametric model (e.g., k-nearest neighbors) and therefore, the predictive cancer model can be trained to make more accurately make predictions without having to optimize parameters.

The exact nature of the biological features evaluated by a particular model (or at least as far as they remain within the confines of the types of biological samples and biological features described herein), and the classification algorithm underlying the particular model, can be generally immaterial to the systems and methods described herein. In some embodiments the output of the model (e.g., disease class model score set 146, as described with respect to step 210 in workflow 200) is a set of continuous or semi-continuous sores. In this fashion, changes occurring with the range of the continuous or semi-continuous scores over time for a subject can be identified (e.g., as delta score set 148, as outlined above relative to step 218 in workflow 200) and evaluated (e.g., against reference delta score sets 154, as outlined above relative to step 200) to classify the disease state of the subject. Accordingly, in some embodiments, the model score set (e.g., first disease class model score set 146-1 and second disease class model score set 146-2) of the model is a likelihood or probability of having the disease condition (318). Similarly, in some embodiments, the model score set (e.g., first disease class model score set 146-1 and second disease class model score set 146-2) of the model is a likelihood or probability of not having the disease condition (320). Thus, a change in the likelihood or probability of having/not having a disease state from a first time point to a second time point can be quantified as a difference in the continuous range of the output.

In some embodiments, e.g., when the disease class evaluation model is a neural network (e.g., a conventional or convolutional neural network), the output of a disease classifier is a classification, e.g., either cancer positive or cancer negative. However, in some embodiments, in order to provide a continuous or semi-continuous value for the output of the model, rather than a classification, a hidden layer of a neural network, e.g., the hidden layer just prior to the output layer, is used as the disease class model score set.

Accordingly, in some embodiments, the model includes (376) (i) an input layer for receiving values for the plurality of genotypic characteristics, where the plurality of genotypic characteristics includes a first number of dimensions, and (ii) an embedding layer that includes a set of weights, where the embedding layer directly or indirectly receives output of the input layer, and where an output of the embedding layer is a model score set having a second number of dimensions that is less than the first number of dimensions, and (iii) an output layer that directly or indirectly receives the model score set from the embedding layer. In such embodiments, the first model score set is the model score set of the embedding layer upon inputting the first genotypic data construct into the input layer, and the second model score set is the model score set of the embedding layer upon inputting the second genotypic data construct into the input layer. In other words, in some embodiments, the model score set is the output of a set of neurons associated with a hidden layer in a neural network termed the embedding layer. In such embodiments, each such neuron in the embedding layer is associated with a weight and an activation function and the model score set comprises the output of each such activation function. In some embodiments, the activation function of a neuron in the embedding layer is rectified linear unit (ReLU), tan h, or sigmoid activation function. In some such embodiments, the neurons of the embedding layer are fully connected to each of the inputs of the input layer. In some such embodiments, each neuron of the output layer is fully connected to each neuron of the embedding layer. In some embodiments, each neuron of the output layer is associated with a Softmax activation function. In some embodiments, one or more of the embedding layer and the output layer is not fully connected.

In some embodiments, each weight in the set of weights of the embedding layer corresponds to a different neuron in a plurality of neurons in the embedding layer. In some such embodiments, the plurality of hidden neurons comprises between two and five hundred, between three and four hundred, between four and three hundred, between five and two hundred, or between six and one hundred neurons. In some embodiments, the plurality of hidden neurons comprises between four neurons and twenty-four neurons.

Generating a Second Disease Model Score Set

As described above with reference to workflow 200, the systems and methods described herein rely on a comparison of disease class model scores generated for two or more biological feature sets for the subject. Accordingly, as indicated in workflow 200, a second iteration of biological sample collection, biological feature set formation, and disease model score set generation are performed. Generally, the same biological features can be used to form the second biological feature set, as well as any subsequent biological feature sets used for analysis of a series of samples. In some embodiments, the biological feature sets include genomic features acquired from nucleic acid samples from the subject. However, as described herein, the systems and methods described herein are not limited to genomic features and may also include, for example, transcriptomic features, epigenetic features, proteomic features, metabolomic features, etc.

Accordingly, in some embodiments, method 300 includes determining (338) a second genotypic data construct (e.g., genotypic data construct 124-2) for the test subject. The second genotypic data construct can include values for the plurality of genotypic characteristics (e.g., the same one or more of read counts 126, allele statuses 130, allelic fractions 134, and methylation statuses 138 included in first genotypic data construct 124-1) based on a second plurality of sequence reads, in electronic form, of a second plurality of nucleic acid molecules in a second biological sample obtained from the test subject at a second test time point occurring after the first test time point (e.g., as outlined above with respect to a second iteration of step 208 or workflow 200).

In some embodiments, the second time point is at least a month after the first time point. In some embodiments, the second time point is at least three months after the first time point. In some embodiments, the second time point is at least 6 months after the first time point. In some embodiments, the second time point is at least 12 months after the first time point. In yet other embodiments, the second time point is at least 2 weeks, 3 weeks, 1 month, 2 months, 3 months, 4 months, 5 months, 6 months, 9 months, or 12 months after the first time point.

In some embodiments, the systems and methods provided herein find use in a periodic monitoring procedure. For example, in some embodiments, a subject provides a biological sample, such as a saliva sample, blood sample, or other liquid sample, on a routine basis, e.g., monthly, which is analyzed according to a method described herein to monitor for development of a disease state in the subject, e.g., cancer. In some embodiments, the subject provides a biological sample about every three months. In some embodiments, the subject provides a biological sample about every six months. In some embodiments, the subject provides a biological sample about annually. In some embodiments, the subject provides a biological sample about every two years.

In some embodiments, a model score (e.g., a first model score) generated at a current time point is used to determine a time span between the current time point and subsequent time points (e.g., six months from the current time point). For example, a subject provides a biological sample, such as a saliva sample, blood sample, or other liquid sample, which is analyzed according to a method described herein to infer a disease condition (e.g., cancer) in the subject. In this situation, for the model score that is close to but below a predetermined threshold, a more frequent periodic monitoring interval (e.g., every three months instead every year for other individuals) can be used.

Accordingly, in some embodiments, the step of inputting a first genotypic data construct into a model for the disease condition, to generate a first model score set for the disease condition, is performed before a second biological sample is obtained from the test subject (between the first and second time points). In some such embodiments, the model score set is evaluated to determine when a follow-up screening should occur for the test subject. For instance, in some embodiments, when the model score set indicates that the subject has a low probability of developing the disease condition (e.g., cancer) within a period of time (e.g., 6 months, 12 months, 18 months, 24 months, 3 years, 4 years, 5 years, 10 years, 15 years, 20 years, or longer), the test subject is provided with a recommendation to repeat testing at a time point that is further away than a recommendation provided to a subject who's model score set indicates a higher probability of developing the disease condition within the period of time. Accordingly, in one embodiment, the disclosure provides a method of determining whether a test subject has a disease condition that includes: (a) determining a first genotypic data construct for the test subject, the first genotypic data construct comprising values for a plurality of genotypic characteristics based on a first plurality of sequence reads, in electronic form, of a first plurality of nucleic acid molecules in a first biological sample obtained from the test subject at a first test time point; (b) inputting the first genotypic data construct into a model for the disease condition, thereby generating a first model score set for the disease condition; (c) evaluating the first model score set to determine a second time test time point, e.g., based upon a risk model for development of the disease condition over time; (d) determining a second genotypic data construct for the test subject, the second genotypic data construct comprising values for the plurality of genotypic characteristics based on a second plurality of sequence reads, in electronic form, of a second plurality of nucleic acid molecules in a second biological sample obtained from the test subject at the second test time point occurring after the first test time point; (e) inputting the second genotypic data construct into the model, thereby generating a second model score set for the disease condition; (f) determining a test delta score set based on a difference between the first and second model score set; and (g) evaluating the test delta score set against a plurality of reference delta score sets, thereby determining whether the test subject has the disease condition, wherein each reference delta score set in the plurality of reference delta scores sets is for a respective reference subject in a plurality of reference subjects.

Accordingly, as outlined above with respect to a second iteration of step 210 of workflow 200, method 300 includes imputing (346) the second genotypic data construct 124-2 into the model (e.g., the same disease classification model 142 as used to evaluate the first genotypic data construct 124-1), to generate a second model score set for the disease condition. The disease classification model used to evaluate the second genotypic data structure may vary slightly, e.g., as it continues to be refined, from the disease classification model used to evaluate the first genotypic data structure. When a particular disease classification model has been refined, or replaced by a different (e.g., improved) disease classification model, that the first genotypic construct, or a refined version of the first genotypic data construct, can be evaluated by the refined or replacing disease classification model, such that the resulting first and second disease class model score sets 146-1-1 and 146-1-2 are more comparable.

Determining a Test Delta Score Set

As outlined above with reference to step 218 of workflow 200, method 300 includes a step of evaluating a change in the disease model score set over time, e.g., between the first disease model score set corresponding to the disease state of the subject at the first time point and the second disease model score set corresponding to the disease state of the subject at the second time. Accordingly, method 300 includes determining (348) a test delta score set (e.g., delta score set 148) based on a difference between the first and second disease model score sets (e.g., disease class model score sets 146-1-1 and 146-1-2).

In some embodiments, the test delta score set is a value or matrix of values corresponding to the raw difference in the value(s) of the two disease model score sets. In some embodiments, the test delta score set is further normalized, prior to evaluation against a distribution of test delta score sets from a reference population. Examples of the types of normalizations contemplated are described in the following section.

Evaluating a Test Delta Score Set

As outlined above with reference to step 220 of workflow 200, method 300 includes a step of evaluating the change in the disease model score set over time (e.g., evaluating delta score set 148), e.g., to determine whether there is a significant change in the disease model score set indicative that the subject is afflicted with the disease state. That is, in some embodiments, method 300 includes a step of evaluating (360) the test delta score set (e.g., delta score set 148) against a plurality of reference delta score sets (e.g., reference delta score sets 152), thereby determining the disease condition of the test subject. Each reference delta score set (e.g., reference delta score set 154) in the plurality of reference delta scores sets can be for a respective reference subject in a plurality of reference subjects.

Generally, referring to method 300, the systems and methods described herein can evaluate whether a change in the disease model score for the test subject over time is significantly different from the types of changes in disease model scores observed over time for reference subjects who do not have the disease state. If the change in the disease model score for the test subject is statistically similar to changes in disease model scores for those reference subjects, than the test subject can be confidently classified as not having the disease state. However, if the change in the disease model score for the test subject is different with statistical significance (e.g., a p-value of 0.05, 0.01, 0.005, etc.), than changes in disease model scores for the reference subjects that don't have the disease condition, it can be inferred that the test subject has a different disease state, that is, the subject likely has the disease state or is developing the disease state. In some embodiments, this comparison is made by generating a distribution of changes in disease model scores for a plurality of reference subjects (e.g., a distribution of reference delta score sets 152) and asking, e.g., using a statistical hypothesis test, whether the change in disease model score for the test subject (e.g., delta score set 148) is a member of that distribution (or in the case of a statistical hypothesis test, whether the test delta score set is not a member of that distribution via a null hypothesis).

Accordingly, in some embodiments, the first model score set (e.g., disease class model score set 146-1) includes a probability that the test subject has the disease condition at the first test time point and the second model score set (e.g., disease class model score set 146-1) includes a probability that the test subject has the disease at the second test time point (e.g., as determined using a disease classification model 142). Accordingly, the test delta score set (e.g., delta score set 148) can include a change in the probability that the test subject has the disease state at the second time point, relative to their probability of having the disease state at the first time point. The test delta score set can be compared (362) to a distribution of the reference delta score sets (e.g., reference delta score sets 146), where each reference delta score set (e.g., each reference delta score set 154) in the plurality of reference delta scores can be for a respective reference subject in the plurality of reference subject based on a difference between (i) a first probability that the respective reference subject has the disease condition provided by the model (e.g., the same disease class evaluation model as used to evaluate the biological features of the test subject) using a first respective reference genotypic data construct including values for the plurality of genotypic features (e.g., the same genotypic features as used for the test subject), taken using a first respective biological sample acquired at a respective first time point from the respective reference subject, and (ii) a second probability that the respective reference subject has the disease condition provided by the model using a second respective genotypic data construct including values for the plurality of genotypic features, taken using a second respective biological sample acquired from the respective reference subject at a respective second time point occurring after the first respective time point, and wherein the respective training subject is free of the disease condition during at least the first and second respective time points.

In some aspects, the present disclosure is based on, at least in part, the recognition that accounting for personal characteristics of the test subject can improve the sensitivity and specificity of methods for classifying a disease state in the test subject. That is, because personal characteristics of the test subject affect the manifestation of the disease state biological signature of the test subject. As such, accounting for one or more of these personal characteristics of the test subject can further improve the sensitivity and specificity of the disease state classification. For instance, the magnitude of the change between the first disease class model score set and the second disease class model score set, as well as the significance of the change, can be affected by at least (i) changes in the disease state of the test subject, e.g., development and progression of the disease state can increase the magnitude of the disease class model score set while regression of the disease state can decrease the magnitude of the disease class model score set, (ii) background variance in the biological characteristics that constitute the disease state signature of the subject, (iii) personal characteristics of the test subject, e.g., age, gender, ethnicity, smoking status, alcohol consumption, familial history, etc., and (iv) the length of time between the first time point (e.g., the time at which the first biological sample was obtained from the test subject) and the second time point (e.g., the time at which the second biological sample was obtained from the test subject), e.g., a 10 percent increase in the probability the subject has a particular disease state is less significant if the length of time between sample collection events is twenty years than if the time between sample collection events is two months.

For instance, background variance refers to a natural fluctuation in a biological property of a subject, e.g., a genotypic characteristic such as methylation. For instance, in some embodiments, the methylation status of an individual's genome may fluctuate up or down from a baseline state over time in a fashion that is unrelated to a particular state of the individual, such as a cancer status. In this fashion, a range for a value of a particular biological characteristic (such as the methylation status of one or more regions of the individual's genome) can be observed from a plurality of samples collected from the individual at different times, even when the individual's health state (e.g., cancer status) does not change. In some instances, the range in the value of the biological characteristic for a first individual can be different than the range of the value of the biological characteristic for a second individual, representing a different level of background variation in the value of the biological characteristic for the first and second individuals.

Accordingly, in some embodiments, one or more of factors affecting the magnitude and/or significance of the change between the first disease class model score set and the second disease class model set are accounted for when evaluating the test delta score set for the test subject against the distribution of reference delta score sets. In some embodiments, these features are accounted for by adjusting or normalizing either, or both, of the test delta score set and the distribution of reference delta score sets. In some embodiments, the adjustment or normalization is applied to the test delta score set and/or the reference delta score sets directly, e.g., each reference delta score set is adjusted or normalized independent of each other. In some embodiments, adjustment or normalization is applied to the reference delta score sets through the reference distribution, e.g., individual reference delta score sets are adjusted or normalized as a function of the distribution, rather than on an individualized basis. In some embodiments, the underlying biological feature data, which is evaluated by the disease classification model, is adjusted or normalized.

In some embodiments, the length of time between collection of the first and second biological samples from the test subject and/or reference subject is used for adjustment or normalization, e.g., the test subject and/or reference subject biological data, and/or the test subject and/or reference subject delta score sets, and/or the distribution of reference delta score sets are adjusted or normalized to account for the time between test subject sample collections.

Accordingly, in some embodiments, an amount of time between the respective first time point and the respective second time point for each respective reference subject in the plurality of reference subjects is used as a covariate (350) in calculating the distribution (e.g., the distribution of reference delta score sets 152). The test delta score set (e.g., delta score set 148) can then be adjusted based on the covariate representing a difference in time between the first test time point and the second test time point for the test subject. In some embodiments, the covariate representing a difference in time between the first test time point and the second test time point (e.g., the length of time between test biological sample collection) is applied to one or more genotypic characteristics in the plurality of characteristics of the first genotypic data construct (e.g., genotypic data construct 142-1-1), the second genotypic data construct (e.g., genotypic data construct 142-1-1), each first respective reference genotypic data construct (e.g., reference genotypic data constructs representing the first time point in the generation of the reference delta score sets 152), or each second respective reference genotypic data construct (e.g., reference genotypic data constructs representing the second time point in the generation of the reference delta score sets 152). In some embodiments, the covariate representing a difference in time between the first test time point and the second test time point is applied to the test delta score set (e.g., delta score set 148) and each reference delta score set (e.g., reference delta score sets 148) in the distribution of reference delta scores.

Similarly, in some embodiments, each respective reference delta score set in the plurality of reference delta scores sets is normalized for an amount of time between the respective first time point and the respective second time point for the respective subject, and the test delta score set is normalized for an amount of time between the first test time point and the test second time point. Likewise, in some embodiments, each respective reference delta score set in the plurality of reference delta score sets is normalized for an amount of time between the respective first time point and the respective second time point for the respective reference subject by normalizing one or more genotypic characteristics in the plurality of characteristics of each first respective reference genotypic data construct or each second respective reference genotypic data construct for an amount of time between the respective first time point and the respective second time point for the respective subject. The test delta score set can be normalized for an amount of time between the first test time point and the test second time point by normalizing one or more genotypic characteristics in the first genotypic data construct and the second genotypic data construct for an amount of time between the first test time point and the second test time point. In some embodiments, the normalizing is applied to the test delta score set and each reference delta score set in the distribution of the reference delta score sets.

In some embodiments, the age of the test and/or reference subject is used for adjustment or normalization, e.g., the test subject and/or reference subject biological data, and/or the test subject and/or reference subject delta score sets, and/or the distribution of reference delta score sets are adjusted or normalized to account for the age of the test subject.

Accordingly, in some embodiments, an age of each respective reference subject in the plurality of reference subjects is used as a covariate (352) in calculating the distribution (e.g., the distribution of reference delta score sets 152). The test delta score set (e.g., delta score set 148) can then be adjusted based on an age of the test subject. In some embodiments, the covariate representing the age of the test subject is applied to one or more genotypic characteristics in the plurality of characteristics of the first genotypic data construct (e.g., genotypic data construct 142-1-1), the second genotypic data construct (e.g., genotypic data construct 142-1-1), each first respective reference genotypic data construct (e.g., reference genotypic data constructs representing the first time point in the generation of the reference delta score sets 152), or each second respective reference genotypic data construct (e.g., reference genotypic data constructs representing the second time point in the generation of the reference delta score sets 152). In some embodiments, the covariate representing the age of the test subject is applied to the test delta score set (e.g., delta score set 148) and each reference delta score set (e.g., reference delta score sets 148) in the distribution of reference delta scores.

Similarly, in some embodiments, each respective reference delta score set in the plurality of reference delta score sets is normalized for an age of the respective reference subject (e.g., age is used as a covariate), and the test delta score set is normalized for an age of the test subject. Each respective reference delta score set in the plurality of reference delta score sets can be normalized for an age of the respective reference subject by normalizing one or more genotypic characteristics in the plurality of characteristics of each first respective reference genotypic data construct or each second respective reference genotypic data construct for the age of the respective subject, and the test delta score set can be normalized for age of the test subject. In some embodiments, the normalizing is applied to the test delta score set and each reference delta score set in the distribution of the reference delta score sets.

In some embodiments, a smoking status or an alcohol consumption characteristic of the test and/or reference subject is used for adjustment or normalization, e.g., the test subject and/or reference subject biological data, and/or the test subject and/or reference subject delta score sets, and/or the distribution of reference delta score sets are adjusted or normalized to account for the smoking status or alcohol consumption characteristic of the test subject.

Accordingly, in some embodiments, a smoking status or an alcohol consumption characteristic of each respective reference subject in the plurality of reference subjects is used as a covariate (354) in calculating the distribution (e.g., the distribution of reference delta score sets 152). The test delta score set (e.g., delta score set 148) can then be adjusted based on a smoking status or an alcohol consumption characteristic of the test subject. In some embodiments, the covariate representing the smoking status or alcohol consumption characteristic of the test subject is applied to one or more genotypic characteristics in the plurality of characteristics of the first genotypic data construct (e.g., genotypic data construct 142-1-1), the second genotypic data construct (e.g., genotypic data construct 142-1-1), each first respective reference genotypic data construct (e.g., reference genotypic data constructs representing the first time point in the generation of the reference delta score sets 152), or each second respective reference genotypic data construct (e.g., reference genotypic data constructs representing the second time point in the generation of the reference delta score sets 152). In some embodiments, the covariate representing the smoking status or alcohol consumption characteristic of the test subject is applied to the test delta score set (e.g., delta score set 148) and each reference delta score set (e.g., reference delta score sets 148) in the distribution of reference delta scores.

Similarly, in some embodiments, each respective reference delta score set in the plurality of reference delta score sets is normalized for a smoking status or an alcohol consumption characteristic of the respective reference subject, and the test delta score set is normalized for a smoking status or an alcohol consumption characteristic of the test subject. Each respective reference delta score set in the plurality of reference delta score sets can be normalized for a smoking status or an alcohol consumption characteristic of the respective reference subject by normalizing one or more genotypic characteristics in the plurality of characteristics of each first respective reference genotypic data construct or each second respective reference genotypic data construct for the smoking status or an alcohol consumption characteristic of the respective subject, and the test delta score set can be normalized for a smoking status or an alcohol consumption characteristic of the test subject. In some embodiments, the normalizing is applied to the test delta score set and each reference delta score set in the distribution of the reference delta score sets.

In some embodiments, a gender/biological sex of the test and/or reference subject is used for adjustment or normalization, e.g., the test subject and/or reference subject biological data, and/or the test subject and/or reference subject delta score sets, and/or the distribution of reference delta score sets are adjusted or normalized to account for the gender of the test subject.

Accordingly, in some embodiments, a gender of each respective reference subject in the plurality of reference subjects is used as a covariate (354) in calculating the distribution (e.g., the distribution of reference delta score sets 152). The test delta score set (e.g., delta score set 148) can then be adjusted based on a gender of the test subject. In some embodiments, the covariate representing the gender of the test subject is applied to one or more genotypic characteristics in the plurality of characteristics of the first genotypic data construct (e.g., genotypic data construct 142-1-1), the second genotypic data construct (e.g., genotypic data construct 142-1-1), each first respective reference genotypic data construct (e.g., reference genotypic data constructs representing the first time point in the generation of the reference delta score sets 152), or each second respective reference genotypic data construct (e.g., reference genotypic data constructs representing the second time point in the generation of the reference delta score sets 152). In some embodiments, the covariate representing the gender of the test subject is applied to the test delta score set (e.g., delta score set 148) and each reference delta score set (e.g., reference delta score sets 148) in the distribution of reference delta scores.

Similarly, in some embodiments, each respective reference delta score set in the plurality of reference delta score sets is normalized for a gender of the respective reference subject, and the test delta score set is normalized for a gender of the test subject. Each respective reference delta score set in the plurality of reference delta score sets can be normalized for a gender of the respective reference subject by normalizing one or more genotypic characteristics in the plurality of characteristics of each first respective reference genotypic data construct or each second respective reference genotypic data construct for the gender of the respective subject, and the test delta score set can be normalized for a gender of the test subject. In some embodiments, the normalizing is applied to the test delta score set and each reference delta score set in the distribution of the reference delta score sets.

In some embodiments, a background variance for a biological characteristic of the test and/or reference subject is used for adjustment or normalization, e.g., the test subject and/or reference subject biological data, and/or the test subject and/or reference subject delta score sets, and/or the distribution of reference delta score sets are adjusted or normalized to account for a background variance for a biological characteristic of the test subject. That is, the amount of variance in the measurement of any particular biological feature may vary from one individual to the next. Accordingly, in some embodiments, a relative level of background variance in measured biological characteristics is determined for the test subject, e.g., by collecting a plurality of biological samples from the subject at a plurality of different times, e.g., 2, 3, 4, 5, 6, 7, 8, 9, 10, or more biological samples. In some embodiments, each sample is collected within 1 day of a previous biological sample, or within 2 days, 3 days, 4 days, 5 days, 6 days, 7 days, two weeks, three weeks, or a month, of a previous biological sample. The intent of collecting these samples may not be to detect changes in the levels of biological features that correlate with progression of the disease state but, rather, to determine the amount of variance in the measurements of biological features from the test subject.

Accordingly, in some embodiments, a background variance for a biological characteristic of each respective reference subject in the plurality of reference subjects is used as a covariate (354) in calculating the distribution (e.g., the distribution of reference delta score sets 152). The test delta score set (e.g., delta score set 148) can then be adjusted based on a background variance for a biological characteristic of the test subject. In some embodiments, the covariate representing the background variance for a biological characteristic of the test subject is applied to one or more genotypic characteristics in the plurality of characteristics of the first genotypic data construct (e.g., genotypic data construct 142-1-1), the second genotypic data construct (e.g., genotypic data construct 142-1-1), each first respective reference genotypic data construct (e.g., reference genotypic data constructs representing the first time point in the generation of the reference delta score sets 152), or each second respective reference genotypic data construct (e.g., reference genotypic data constructs representing the second time point in the generation of the reference delta score sets 152). In some embodiments, the covariate representing the background variance for a biological characteristic of the test subject is applied to the test delta score set (e.g., delta score set 148) and each reference delta score set (e.g., reference delta score sets 148) in the distribution of reference delta scores.

Similarly, in some embodiments, each respective reference delta score set in the plurality of reference delta score sets is normalized for a background variance for a biological characteristic of the respective reference subject, and the test delta score set is normalized for a background variance for a biological characteristic of the test subject. Each respective reference delta score set in the plurality of reference delta score sets can be normalized for a background variance for a biological characteristic of the respective reference subject by normalizing one or more genotypic characteristics in the plurality of characteristics of each first respective reference genotypic data construct or each second respective reference genotypic data construct for the background variance for a biological characteristic of the respective subject, and the test delta score set can be normalized for a background variance for a biological characteristic of the test subject. In some embodiments, the normalizing is applied to the test delta score set and each reference delta score set in the distribution of the reference delta score sets.

In some embodiments, rather than adjusting or normalizing a single distribution of reference delta score sets, a segmented reference distribution is used in which all of the reference subjects are one of an enumerated class of individuals sharing one or more personal characteristics with the test subject. For example, in some embodiments, a reference distribution is selected such that all of the reference subjects used in the reference distribution have a similar age as the test subject. In some embodiments, system 100 stores a plurality of segmented reference distributions, or forms a segmented reference distribution based on one or more personal attributes of the test subject. In some embodiments, each reference subject in a segmented distribution has an age, gender, smoking status, background variance in a biological characteristic, and/or alcohol consumption characteristic that is shared with the test subject. Accordingly, in some embodiments, the plurality of reference subjects is segmented for gender, age, smoking status, alcohol consumption, background variance in a biological characteristic, or a combination thereof (3074). For instance, a segmented reference distribution can be formed from the reference delta score sets 154 that share one or more enumerated personal characteristic with the test subject.

In some embodiments, to account for the variance of biological characteristics in the test subject, a plurality of baseline genotypic data constructs for the test subject are determined (358). Each respective baseline genotypic data construct in the plurality of baseline genotypic data constructs can include values for the plurality of genotypic characteristics (e.g., the same one or more of read counts 126, allele statuses 130, allelic fractions 134, and methylation statuses 138 used to form the genotypic data construct 124 and corresponding reference genotypic data constructs) based on a corresponding baseline plurality of sequence reads, in electronic form, of a corresponding plurality of nucleic acid molecules in a corresponding baseline biological sample, in a plurality of baseline biological samples, obtained from the test subject at a corresponding baseline test time point occurring before the second test time point (e.g., prior to obtaining the first biological sample, or after obtaining the first biological sample). In some embodiments, the first biological sample is used as one of the baseline biological samples for the test subject. Then, an amount of variance in values for one or more respective genotypic characteristic, in the plurality of genotypic characteristics, between respective baseline genotypic data constructs in the plurality of baseline genotypic constructs can be used to calculate a baseline variance covariate specific to the test subject. This baseline covariate can be applied to the distribution of the reference delta score sets, to normalize the distribution of the reference delta score sets against the baseline variability of the test subject.

In some embodiments, the test delta score set (e.g., test delta score set 148) is evaluated by performing a statistical hypothesis test against a reference distribution of delta score sets (e.g., reference delta score sets 152) from reference subjects that are not afflicted with the disease state, which may or may not be adjusted or normalized to account for a covariate. In some embodiments, the statistical hypothesis test provides a measure of statistical significance for whether or not the test delta score set is a member of the distribution of reference delta score sets. In some embodiments, the subject is deemed to be afflicted with the disease state when the statistical hypothesis test provides a one-tailed p-value that satisfies a threshold level of significance, e.g., p=0.05, 0.1, 0.005, etc. In some embodiments, the one-tailed test is used because negative changes in the disease class model score set indicate that the disease is regressing in the subject, rather than progressing. Thus, outliers on the high end of the distribution can be determined to have the disease state.

In a related methodology, in some embodiments, the test delta score set (e.g., test delta score set 148) is evaluated by determining whether the test delta score set falls within a rejection region of the reference distribution. For example, a rejection region of the reference distribution of delta score sets (e.g., reference delta score sets 152) can be defined by selecting a significance level (e.g., an alpha level setting an acceptable probability of an error supporting the alternative hypothesis—that a subject does not have a disease condition—when the null hypothesis—that the subject does have the disease condition—is true), and then it is determined whether the test delta score set (e.g., test delta score set 148) falls within the rejection region of the reference distribution.

Accordingly, in some embodiments, the comparison between the test delta score set and the distribution of reference delta score sets includes determining (364) a measure of central tendency of the distribution (e.g., the distribution of reference delta score sets 152) and a measure of spread of the distribution. Then, the comparison can include determining a significance of the test delta score set using the measure of central tendency of the distribution and the measure of spread of the distribution. In some embodiments, the measure of central tendency of the distribution is an arithmetic mean, weighted mean, midrange, midhinge, trimean, Winsorized mean, mean, or mode across the distribution (366). In some embodiments, the measure of spread of the distribution is a standard deviation, a variance, or a range of the distribution (368).

In some embodiments, the measure of central tendency of the distribution is the mean of the distribution, the measure of spread of the distribution is the standard deviation of the distribution, and the determining the significance of the test delta score set using the measure of central tendency of the distribution and the measure of spread of the distribution comprises determining a number of standard deviations the test delta score set is from the mean of the distribution (370). In some embodiments, the test subject is determined to have the disease condition when the number of standard deviations the test delta score set from the mean of the distribution satisfies a threshold value (372). That is, it can be expected that the test subject does not have the disease condition (e.g., cancer or coronary disease condition) if their delta score set is similar to those in the distribution.

In some embodiments, the reference distribution of delta score sets (e.g., reference delta score sets 152) is normalized to generate a normal distribution, a t-distribution, a chi-squared distribution, an F-distribution, a lognormal distribution, a Weibull distribution, an exponential distribution, a uniform distribution, or any other normalized distribution.

In some embodiments, the test delta score set is evaluated using a classifier trained against the plurality of reference delta score sets, e.g., rather than by statistical comparison to the distribution of the reference delta score sets. For instance, in some embodiments, the evaluating (378) includes inputting the test delta score into a classifier trained against the plurality of reference delta score sets, where each reference delta score set in the plurality of reference delta scores is for a respective reference subject in the plurality of reference subject based on a difference between (i) a first probability that the respective reference subject has the disease condition provided by the model using a respective first reference genotypic data construct having values for the plurality of genotypic features, taken using a respective first biological sample acquired at a respective first time point from the respective reference subject, and (ii) a second probability that the respective reference subject has the disease condition provided by the model using a respective second genotypic data construct having values for the plurality of genotypic features, taken using a respective second biological sample acquired from the respective reference subject at a respective second time point occurring after the respective first time point, and wherein the respective training subject is free of the disease condition during at least the respective first and second time points.

In some embodiments, the classifier is further trained on whether one or more of the reference subjects later developed the disease condition (e.g., later developed cancer). That is, in some embodiments, each of a plurality of reference subjects are determined not to have the disease condition (e.g., cancer) at respective first and second time points, e.g., as determined using a disease classification model 142 that provides a disease class model score set 146 based on a genotypic data construct 124 determined from a biological sample (e.g., a liquid biological sample). The change in the disease class model score sets over time, e.g., the delta score set 148, is used as an independent variable when training the classifier. Then, some or all of the reference subjects can be further evaluated for the disease condition at a third time point that is after the first and second time point. In some embodiments, the result of that later evaluation, e.g., whether or not the reference subject later developed the disease condition, is used as a dependent variable when training the classifier. In this fashion, particular changes in the disease class model score set 146 over time can be better associated with future outcomes and/or can be used to leverage earlier detection of the disease condition. Accordingly, in some embodiments, the classifier is further trained against, for each respective training subject in at least a subset of the plurality of reference subjects, a determination of whether the respective subject had the disease condition at a respective third time point occurring after the respective second time point.

As described herein with reference to other embodiments, in some embodiments, the amount of time between the respective first, second, and third time points, as well as non-genotypic characteristics of the reference subject, are used to normalize the data. That is, these characteristics can be used as co-variates when determining values for a genotypic data construct, a disease class model score set, or a delta score set, e.g., prior to training the classifier. In some embodiments, one or more of these characteristics are further used to train the classifier.

In some embodiments, the classifier is a neural network algorithm, a support vector machine algorithm, a Naive Bayes algorithm, a nearest neighbor algorithm, a boosted trees algorithm, a random forest algorithm, a decision tree algorithm, a multinomial logistic regression algorithm, or a linear regression algorithm, as described elsewhere herein.

In some embodiments, the test delta score set is evaluated by logistic regression, rather than statistics. For instance, in some embodiments, the evaluating (378) includes evaluating the test delta score set using a logistic function trained by logistic regression against the plurality of reference delta score sets.

In some embodiments, each reference delta score set in the plurality of reference delta scores is for a respective reference subject in the plurality of reference subjects based on a difference between: (i) a first score set provided by the embedding layer of the model using a first respective reference genotypic data construct comprising values for the plurality of genotypic features, taken using a first respective biological sample acquired at a respective first time point from the respective reference subject, and (ii) a second score set provided by the embedding layer of the model using a second respective genotypic data construct comprising values for the plurality of genotypic features, taken using a second respective biological sample acquired from the respective reference subject at a respective second time point other than the first respective time point. In some embodiments, the model is a convolutional neural network (380). In some embodiments, a first subset of the plurality of reference subjects have the disease condition and a second subset of the plurality of reference subjects do not have the disease condition (382). In some embodiments, each reference subject in the plurality of reference subjects does not have the disease condition (384).

In some embodiments, the logistic regression further includes personal characteristics, for example one or more of gender, age, smoking status, and alcohol consumption, in order to account for such characteristics, as described above for the statistical methods.

The regression algorithm can be any type of regression. For example, in some embodiments, the regression algorithm is logistic regression. In some embodiments, the logistic regression assumes:

$$P ( {x}_{i} ) = \frac{exp ( {β}_{0} + {β}_{1} {x}_{i 1} + … + {β}_{k} {x}_{ik} )}{1 + exp ( {β}_{0} + {β}_{1} {x}_{i 1} + … + {β}_{k} {x}_{ik} )} ,$$

where:
   xᵢ=(xᵢ₁, xᵢ₂, . . . , xᵢₖ) are the corresponding biological feature values (e.g., one or more of read counts 126, allele statuses 130, allelic fractions 134, and methylation statuses 138), obtained from biological samples for the i^{th} corresponding training subject, where the i^{th} corresponding training subject either has a first disease status (e.g., cancer condition or coronary disease) (Y=1) or a second disease status (Y=0);
   Y∈{0, 1} is a class label that has the value “1” when the corresponding subject i has the first disease status and has the value “0” when the corresponding subject i has the second disease status,
      β₀ is an intercept, and
   βⱼ=(j=1, . . . k) is a plurality of regression coefficients, where each respective regression coefficient in the plurality of regression coefficients is for a corresponding biological feature value.

In some embodiments, the logistic regression is logistic least absolute shrinkage and selection operator (LASSO) regression. In such embodiments, the logistic LASSO estimator [symbol] , . . . , [symbol] is defined as the minimizer of the negative log likelihood:

min(Σᵢ₌₁ⁿ[−yᵢ(β₀+β₁xᵢ+ . . . +βₖxᵢₖ)+log(1+exp(β₀+β₁xᵢ+ . . . +βₖxᵢₖ))]),

subject to the constraint Σⱼ₌₁^{k}=|βⱼ|≤λ, where λ is a constant optimized for any given dataset.

In some embodiments, the regression algorithm is logistic regression with lasso, L2 or elastic net regularization.

As noted in the above equations, each xᵢ=(xᵢ₁, xᵢ₂, . . . , xᵢₖ) are the corresponding feature values for the i^{th} corresponding training subject and, as such, each xᵢ, represents a corresponding biological feature. Moreover, each βⱼ=(j=1, . . . k) is the regression coefficient for a corresponding biological feature. In some embodiments, those extracted features that have a corresponding regression coefficient that fails to satisfy a threshold value are pruned (removed from) the plurality of biological features. In some embodiments, this threshold value is zero. Thus, in such embodiments, those biological features that have a corresponding regression coefficient that is zero from the above-described regression are removed from the plurality of biological features prior to training the classifier. In some embodiments, for instance, in which L2 regularization is employed, the threshold value is 0.1. Thus, in such embodiments, those biological features that have a corresponding regression coefficient whose absolute value is less than 0.1 from the above-described regression are removed from the plurality of extracted features prior to training the classifier. In some embodiments, the threshold value is a value between 0.1 and 0.3. An example of such embodiments is the case where the threshold value is 0.2. In such embodiments, those extracted features that have a corresponding regression coefficient whose absolute value is less than 0.2 from the above-described regression are removed from the plurality of extracted features prior to training the classifier.

Method 400

In one aspect, the disclosure provides a method 400 that uses a population distribution to classify the disease state of a test subject based on changes in the probability or likelihood that the test subject has the disease state over a series of measurements, as determined using a classifier trained to distinguish the disease state from one or more other disease states. Method 400 relates directly to the descriptions of disease states, methods for obtaining biological samples, and methods for obtaining biological features described above. Further, many of the features and processes involved in method 400 can be the same as for method 300, described above. For brevity, description of some of these features is not repeated below. However, any of the features and processes described above, e.g., with reference to method 300, can also be applicable to method 400.

Referring generally to FIGS. 4A-4F, in some embodiments, the method includes determining, for each respective test time point in a plurality of test time points, a corresponding genotypic data construct (e.g., genotypic data constructs 124) for the test subject (e.g., as outlined above with reference to several iterations of step 208 of workflow 200). The corresponding genotypic data construct can include values for a plurality of genotypic characteristics (e.g., one or more of read counts 126, allele statuses 130, allelic fractions 134, and methylation statuses 138) based on a corresponding plurality of sequence reads, in electronic form (e.g., cfDNA sequence reads generated at corresponding iterations of step 206 of workflow 200), of a corresponding plurality of nucleic acid molecules in a corresponding biological sample obtained from the test subject at the respective test time point (e.g., a sample obtained at corresponding iterations of step 204 of workflow 200). The method can include inputting the corresponding genotypic data construct (e.g., of genotypic data constructs 124) into a model (e.g., disease classification model 142) for the disease condition to generate a corresponding time stamped model score set (e.g., of disease class model score sets 146-1) for the disease condition at the respective test time point, thereby obtaining a plurality of time stamped test model score sets for the test subject (e.g., disease class model score sets 146-1-1 through 146-1-N), where each respective time stamped test model score set is coupled to a different test time point in the plurality of test time points (e.g., different iterations of the data collection and analysis workflow). The method can include fitting the plurality of time stamped test model score sets with a temporal trend test (e.g., as outlined above with reference to step 218 of workflow 200), thereby obtaining a temporal test trend parameter set for the test subject (e.g., temporal test trend parameter 149-1). The method can include evaluating the test trend parameter set for the test subject (e.g., as outlined above with reference to step 220 of workflow 200) against a plurality of reference trend parameter sets (e.g., as analogized to reference delta score sets 152) for a plurality of reference subjects thereby determining the disease condition of the test subject (e.g., test subject classification 162), where each respective reference trend parameter set in the plurality of reference trend parameter sets is for a corresponding reference subject in the plurality of reference subjects.

Advantageously, by collecting a series of biological samples for the test subject over time, the personal variance in biological characteristics of the subject can be better accounted for when monitoring for a disease state. For instance, some subjects can inherently demonstrate a greater variance in biological characteristics. In these subjects, a small shift in a determined probability that the subject has a particular disease state can be less informative than in subjects having less variance in biological characteristics. That is, it is expected, when monitoring subjects demonstrating higher variance in biological characteristics for a disease condition over time, that the probability of the subject having the disease state can fluctuate more, e.g., both in the positive and negative directions. As such, a small increase in a determined probability that the subject has a disease state can be likely explained by the natural variance in their biological characteristics, rather than by an underlying biological response to development of the disease state. In contrast, a small increase in a determined probability that a subject having little variance in their biological characteristics has a disease state can be less likely to be explained by natural variance, and can be more likely indicative of a biological response associated with development of the disease state. Conventional methods for classifying a disease state in a subject cannot account for personal variance in a subject's biological characteristics, because they use data for a single time point. Advantageously, in some embodiments, the systems and methods described herein improve upon these convention methods for classifying a disease state by accounting for personal variance.

Accordingly, in some embodiments, method 400 uses biological information from a series of samples collected over a plurality of test time points. In some embodiments, the plurality of test time points is three or more time points (436). In some embodiments, the plurality of test time points is four or more time points. In some embodiments, the plurality of test time points is ten or more time points. In yet other embodiments, the plurality of test time points is at least 3, 4, 5, 6, 7, 8, 9, 10, 15, 20, or more test time points.

In some embodiments, the plurality of test time points span a period of months or years (438). For instance, in some embodiments, the plurality of test time points spans at least six months. In some embodiments, the plurality of test time points spans at least a year. In some embodiments, the plurality of test time points spans at least five years. In yet other embodiments, the plurality of test time points spans at least 6 months, 7 months, 8 months, 9 months, 10 months, 11 months, 1 years, 2 years, 3 years, 4 years, 5 years, 6 years, 7 years, 8 years, 9 years, 10 years, 15 years, 20 years, or longer.

In some embodiments, the plurality of test time points form an unevenly spaced time series (440). For instance, in some embodiments, biological samples are collected from the subject when they visit a medical facility (e.g., doctor's office, hospital, clinic, medical laboratory, etc.), e.g., for an unrelated reason. In other embodiments, the plurality of test time points form a more evenly spaced time series. For instance, in some embodiments, biological samples are collected from the subject on a monthly, semi-annual, or annual basis, e.g., via regularly scheduled visits to a medical facility or by remote sample submission.

Generating Biological Feature Sets

As outlined above with reference to step 208 of workflow 200, method 400 includes steps of generating biological feature set (e.g., genotypic data construct 124) from biological characteristics obtained from a plurality of biological samples, obtained over a series of time from the test subject. The particular features included in, and the formatting of, the biological feature sets can be dictated by the classifier used (e.g., disease classification model 142) to determine an initial probability or likelihood that a particular disease state (e.g., cancer, a type of cancer, a cardiovascular disease, etc.). In some embodiments, the classifier uses genotypic features obtained from sequence reads acquired from a nucleic acid containing sample from the subject (e.g., a liquid sample containing cfDNA).

Accordingly, in some embodiments, a respective feature set includes features determined from a respective plurality of nucleic acids in a respective biological sample obtained from the subject. In some embodiments, the respective plurality of nucleic acids include DNA molecules (e.g., cfDNA or genomic DNA). In some embodiments, the respective plurality of nucleic acids include RNA molecules (e.g., mRNA). In some embodiments, the respective plurality of nucleic acids include both DNA and RNA molecules.

Accordingly, in some embodiments, method 400 includes, for each respective test time point (402) in a plurality of test time points, determining (404) a corresponding genotypic data construct for a test subject, the corresponding genotypic data construct including values for a plurality of genotypic characteristics based on a corresponding plurality of sequence reads (e.g., sequence reads obtained as described above with reference to step 206 illustrated in FIG. 2), in electronic form, of a corresponding plurality of nucleic acid molecules in a corresponding biological sample obtained from the test subject at the respective test time point

In some embodiments, the test subject is a human (406). In some embodiments, the test subject (e.g., a human) has not been diagnosed as having the disease condition (408). For instance, in some embodiments, the methods described herein find utility in being able to identify a disease state in a subject before a biological signature for the disease reaches a level of detection (LOD) for a conventional classifier. Accordingly, in some embodiments, the subject has been tested for the disease state multiple times, and each time has been classified as not having the disease state.

In some embodiments (410), the plurality of genotypic characteristics include one or more characteristics including support for a single nucleotide variant at a genetic location (e.g., allele status 130), a methylation status at a genetic location (e.g., regional methylation status 138), a relative copy number for a genetic location (e.g., bin read count 126), an allelic ratio for a genetic location (e.g., allelic fraction 134), a fragment size metric of the cell-free nucleic acid molecules, a methylation pattern at a genetic location, and a mathematical combination thereof

In some embodiments, the plurality of genotypic characteristics include a plurality of relative copy numbers (e.g., bin read counts 126), where each respective relative copy number in the plurality of relative copy numbers corresponds to a different genetic location in a plurality of genetic locations (412). In some embodiments, the relative copy numbers represent the relative abundance of sequence reads from a plurality of genomic regions. In some embodiments, the genomic regions have the same size. In some embodiments, the genomic regions have different sizes. As described above, with reference to method 300, in some embodiments, the copy number data is further normalized, e.g., to reduce or eliminate variance in the sequencing data caused by potential confounding factors.

In some embodiments, the plurality of genotypic characteristics includes a plurality of methylation statuses (e.g., regional methylation statuses 138), where each methylation status in the plurality of methylation statuses corresponds to a different genetic location in a plurality of genetic locations (414). In some embodiments, each methylation status is represented by a methylation state vector as described, for example, in U.S. Provisional Patent Application No. 62/642,480, entitled “Methylation Fragment Anomaly Detection,” filed Mar. 13, 2018, which is hereby incorporated by reference herein in its entirety. As described above, with reference to method 300, in some embodiments, the methylation data is normalized, e.g., to reduce or eliminate variance in the sequencing data caused by potential confounding factors.

However, as described herein, in some embodiments, a particular classification model evaluates features other than genomic characteristics, e.g., instead of, or in addition to, the genomic characteristics described above. For instance, in some embodiments, the classification model evaluates epigenetic markers (epigenetics), gene expression profiling (transcriptomics), protein expression or activity profiling (proteomics), metabolic profiling (metabolomics), etc. Accordingly, in some embodiments, the biological feature sets formed include one or more of these non-genomic biological features.

Additionally, in some embodiments, the classification model evaluates one or more personal characteristics of the subject, e.g., gender, age, smoking status, alcohol consumption, familial history, etc., in addition to the biological features. Accordingly, in some embodiments, the biological feature sets formed includes one or more personal characteristics of the subject.

Generating Disease Model Score Sets

As outlined above with reference to step 210 of workflow 200, method 400 includes using the biological feature set formed from the biological characteristics obtained from the biological samples of the subject over time to generate a series of disease model score sets. Accordingly, in some embodiments, method 400 includes, for each respective test time point in a plurality of test time points, inputting (416) the corresponding genotypic data construct (e.g., a genotypic data construct 124) into a model for a disease condition (e.g., disease classification model 142), thereby generating a corresponding time stamped model score set (e.g., a disease class model score set 146) for the disease condition at the respective test time point, thereby obtaining a plurality of time stamped test model score sets for the test subject. Each respective time stamped test model score set can be coupled to a different test time point in the plurality of test time points. Generally, the identity and type of disease model used by the systems and methods described herein can be immaterial.

Many different models that evaluate biological features in order to classifying one or more disease statuses (e.g., a cancer status, coronary disease status, etc.) of a subject have been developed. For instance, U.S. Patent Application Publication No. 2019/0287652 describes models that evaluate the methylation status across a plurality of genomic loci, e.g., using cfDNA samples, in order to classify a cancer status of a subject. Similarly, U.S. Patent Application Publication No. 2019/0287649 describes models that evaluate the relative copy number across a plurality of genomic loci, e.g., using cfDNA samples, in order to classify a cancer status of a subject. Likewise, various models have been developed that evaluate the presence of variant alleles (e.g., single nucleotide variants, indels, deletions, transversions, translocations, etc.) in order to classify a cancer status of a subject. Generally, any model developed for the classification of a disease status of a subject may be used in conjunction with the systems and methods described herein.

In some embodiments, the model is for detecting the presence of a disease state in a subject, e.g., detecting cancer or coronary disease in a subject. That is, the systems and methods provided herein are particularly well suited for improving upon the sensitivity and specificity of existing disease models, because they facilitate identity of changes in the biological signature of a subject over time, even when the biological signal is not yet strong enough for the underlying model to detect. Accordingly, in some embodiments, the model (e.g., the underlying model used to evaluate a genotypic data construct 124 at step 210 of workflow 200) evaluates data from a single time point. That can be samples that evaluate biological features acquired from a single sample from the subject, or from a plurality of samples acquired at a same or similar point in time from the subject (e.g., samples providing different types of biological information, such as genomic and transcriptomic information).

Generally, many different classification algorithms can find use in the systems and methods described herein. For instance, in some embodiments, the model is a neural network algorithm, a support vector machine algorithm, a Naive Bayes algorithm, a nearest neighbor algorithm, a boosted trees algorithm, a random forest algorithm, a decision tree algorithm, a multinomial logistic regression algorithm, a linear model, or a linear regression algorithm (434), details of which are described elsewhere herein. Generally, the type of classifier used to generate a disease model score set for one or more disease states, using the systems and methods described herein, can be immaterial. In some embodiments, the model is trained (432) on a cohort of subjects in which a first portion of the cohort has the disease condition and a second portion of the cohort is free of the disease condition, e.g., such that it is specifically trained to distinguish between a first state corresponding to not having the disease condition and a second state corresponding to having the disease condition.

In some aspects, the disclosed methods can work in conjunction with cancer classification models (418). For example, a machine learning or deep learning model (e.g., a disease classifier) can be used to determine a disease state based on values of one or more features determined from one or more cell-free DNA molecules or sequence reads (e.g., derived from one or more cfDNA molecules). In various embodiments, the output of the machine learning or deep learning model is a predictive score or probability of a disease state (e.g., a predictive cancer score).

In some embodiments, the machine-learned model includes a logistic regression classifier. In other embodiments, the machine learning or deep learning model can be one of a decision tree, an ensemble (e.g., bagging, boosting, random forest), gradient boosting machine, linear regression, Naïve Bayes, or a neural network. The disease state model can include learned weights for the features that are adjusted during training. The term “weights” is used generically here to represent the learned quantity associated with any given feature of a model, regardless of which particular machine learning technique is used. In some embodiments, a cancer indicator score is determined by inputting values for features derived from one or more DNA sequences (or DNA sequence reads thereof) into a machine learning or deep learning model.

During training, training data can be processed to generate values for features that are used to train the weights of the disease state model. As an example, training data can include cfDNA data, cancer gDNA, and/or WBC gDNA data obtained from training samples, as well as an output label. For example, the output label can be an indication as to whether the individual is known to have a specific disease (e.g., known to have cancer) or known to be healthy (i.e., devoid of a disease). In other embodiments, the model can be used to determine a disease type, or tissue of origin (e.g., cancer tissue of origin), or an indication of a severity of the disease (e.g., cancer stage) and generate an output label therefor. Depending on the particular embodiment, the disease state model can receive the values for one or more of the features determine from a DNA assay used for detection and quantification of a cfDNA molecule or sequence derived therefrom, and computational analyses relevant to the model to be trained. In one embodiment, the one or more features comprise a quantity of one or more cfDNA molecules or sequence reads derived therefrom. Depending on the differences between the scores output by the model-in-training and the output labels of the training data, the weights of the predictive cancer model can be optimized to enable the disease state model to make more accurate predictions. In various embodiments, a disease state model may be a non-parametric model (e.g., k-nearest neighbors) and therefore, the predictive cancer model can be trained to make more accurately make predictions without having to optimize parameters.

While the exact nature of the biological features evaluated by a particular model (or at least as far as they remain within the confines of the types of biological samples and biological features described herein), and the classification algorithm underlying the particular model, can be generally immaterial to the systems and methods described herein, in some embodiments the output of the model (e.g., disease class model score set 146, as described with respect to step 210 in workflow 200) can be a set of continuous or semi-continuous scores. In this fashion, changes occurring with the range of the continuous or semi-continuous scores over time for a subject can be identified (e.g., using trend test parameter 149, as outlined above relative to step 218 in workflow 200) and evaluated (e.g., against reference trend test parameters, as outlined above relative to step 200) to classify the disease state of the subject. Accordingly, in some embodiments, the model score set (e.g., disease class model score sets 146) of the model is a likelihood or probability of having the disease condition (420). Similarly, in some embodiments, the model score set (e.g., disease class model score sets 146) of the model is a likelihood or probability of not having the disease condition. Thus, a change in the likelihood or probability of having/not having a disease state from a first time point to a second time point can be quantified as a difference in the continuous range of the output.

In some embodiments, e.g., when the disease class evaluation model is a neural network (e.g., a conventional or convolutional neural network), the output of a disease classifier is a classification, e.g., either cancer positive or cancer negative. However, in some embodiments, in order to provide a continuous or semi-continuous value for the output of the model, rather than a classification, a hidden layer of a neural network, e.g., the hidden layer just prior to the output layer, is used as the disease class model score set.

Accordingly, in some embodiments, the model includes (i) an input layer for receiving values for the plurality of genotypic characteristics, where the plurality of genotypic characteristics includes a first number of dimensions, and (ii) an embedding layer that includes a set of weights, where the embedding layer directly or indirectly receives output of the input layer, and where an output of the embedding layer is a model score set having a second number of dimensions that is less than the first number of dimension, and (iii) an output layer that directly or indirectly receives the model score set from the embedding layer, where the first model score set is the model score set of the embedding layer upon inputting the first genotypic data construct into the input layer, and the second model score set is the model score set of the embedding layer upon inputting the second genotypic data construct into the input layer.

Determining a Test Trend Parameter Set

As outlined above with reference to step 218 of workflow 200, method 400 includes a step of evaluating a change in the disease model score set over time, e.g., between the plurality of disease model score sets (e.g., disease class model score sets 146-1-1 to 146-1-N) corresponding to the disease state of the subject at each time point in the plurality of test time points in the series. In some embodiments, the evaluation is made using a temporal trend test, for instance, the Cochran-Armitage trend test, the Mann-Kendall test, and the Mann-Whitney U Test.

For example, the Cochran-Armitage trend test evaluates trends in binomial proportions across the levels of a single variable. Briefly, variance Var(T) from the null hypothesis (no association) of the Cochran-Armitage trend statistic:

T≡Σᵢ₌₁^{k}tᵢ(N₁ᵢR₂−N₂ᵢR₁),

where k is the number of categories, tᵢ are weights, Nₖᵢ represents the i^{th} observation of the k^{th} category, and Rₖ represents the sum of the i observations for the k^{th} category, can be calculated as:

$$Var ( T ) = \frac{{R}_{1} {R}_{2}}{N} ( {\sum }_{i = 1}^{k} {t}_{i}^{2} {C}_{i} ( N - {C}_{i} ) - 2 {\sum }_{i = 1}^{k - 1} {\sum }_{j = 1 + 1}^{k} {t}_{i} {t}_{j} {C}_{i} {C}_{{j}_{1 i}} ) .$$

The Mann-Kendall test can be a non-parametric trend test used to identify monotonic trends (one-way trends) in series data. Briefly, the Mann-Kendall test can employ a Kendall rank correlation of consecutive observations (e.g., the series of disease class model score sets 146 determined for a plurality of time points) with time, to test for monotonic trends. The null hypothesis for the test can be that there are no trends. That is, the observations can be independently distributed with respect to the time series. Kendall's tau coefficient can be a statistic used to measure the ordinal association between two measured quantities, e.g., disease class model score sets 146.

Accordingly, in some embodiments, method 400 includes fitting (446) the plurality of time stamped test model score sets (e.g., disease class model score sets 146-1-1 through 146-1-N for the time series), with a temporal trend test (e.g., a Cochran-Armitage trend test, a Mann-Kendall test, a Mann-Whitney U Test, or by log-linear least squares fitting), thereby obtaining a test trend parameter set (e.g., temporal trend test parameter 149) for the test subject. In some embodiments, fitting the time stamped test model score sets is performed by log-linear least squares fitting a plurality of time stamped test model scores of the test subject to obtain the slope of the line for the test subject.

In some embodiments, method 400 also includes fitting a corresponding plurality of reference time stamped time model score sets with the temporal trend test (e.g., the same temporal trend test used to fit the data for the test subject) thereby obtaining a respective reference trend parameter set in a distribution of a plurality of reference trend parameter sets for corresponding reference subject. In some embodiments, the temporal trend test is a Cochran-Armitage trend test, a Mann-Kendall test, a Mann-Whitney U Test, or by log-linear least squares fitting. In some embodiments, the fitting includes log-linear least squares fitting a corresponding plurality of time stamped time points of the corresponding reference subject to obtain the slope of a line for the corresponding reference subject.

Evaluating a Test Trend Parameter Set

As outlined above with reference to step 220 of workflow 200, method 400 includes a step of evaluating the change in the disease model score set over time (e.g., evaluating temporal trend test parameter 149), e.g., to determine whether there is a significant change in the disease model score set indicative that the subject is afflicted with the disease state. That is, method 400 can include a step of evaluating (452) the test trend parameter set (e.g., temporal trend test parameter 149) for the test subject against a plurality of reference trend parameter sets for a plurality of reference subjects (e.g., analogous reference trend test parameters to the reference delta score sets 154 as illustrated in FIG. 1A), thereby determining the disease condition of the test subject, where each respective reference trend parameter set in the plurality of reference trend parameter sets is for a corresponding reference subject in the plurality of reference subjects.

Generally, referring to method 400, in some embodiments the systems and methods described herein evaluate whether a trend in the changes in the disease model score for the test subject over time is significantly different from the types of trends for changes in disease model scores observed over time for reference subjects who do not have the disease state. If the trend for change in the disease model score for the test subject is statistically similar to the trend for changes in disease model scores for those reference subjects, then the test subject can be confidently classified as not having the disease state. However, if the trend for change in the disease model score for the test subject is different with statistical significance (e.g., a p-value of 0.05, 0.01, 0.005, etc.), than the trend for changes in disease model scores for the reference subjects that don't have the disease condition, it can be inferred that the test subject has a different disease state, that is, the subject likely has the disease state or is developing the disease state. In some embodiments, this comparison is made by generating a distribution of trend statistics for changes in disease model scores for a plurality of reference subjects (e.g., analogous to the distribution of reference delta score sets 152, as discussed above with reference to method 300) and asking, e.g., using a statistical hypothesis test, whether the trend for change in disease model score for the test subject (e.g., temporal trend test parameter 149) is a member of that distribution (or in the case of a statistical hypothesis test, whether the trend test parameter is not a member of that distribution via a null hypothesis).

In some embodiments, evaluation of the trend test parameter is done using a parametric statistical hypothesis test. In some embodiments, each timed stamped test model score set in the plurality of timed stamped test model score sets (e.g., disease class model score sets 146-1-1 through 146-1-N for the test subject) includes a probability that the test subject has the disease condition (e.g., cancer or a coronary disease) at the corresponding test time point (4054). Accordingly, the trend test parameter (e.g., temporal trend test parameter 149) can be a statistical measure of whether a trend in the time stamped test model sets exists. The test trend parameter set for the test subject (e.g., temporal trend test parameter 149) can be compared to a distribution formed from a plurality of reference trend parameter sets (e.g., analogous to a distribution of the reference delta score sets 152 shown in FIG. 1A).

Each reference trend parameter set in the plurality of reference trend parameter sets can be for a corresponding reference subject in the plurality of reference subject, and can be determined by, for each respective corresponding reference time point in a corresponding plurality of reference time points associated with the corresponding reference subject, (i) determining a corresponding genotypic data construct for the reference subject, the corresponding genotypic data construct including values for the plurality of genotypic characteristics (e.g., the same genotypic characteristics used to form genotypic data constructs 124 for the test subject) based on a corresponding plurality of sequence reads, in electronic form, of a corresponding plurality of nucleic acid molecules in a corresponding biological sample obtained from the corresponding reference subject at the corresponding time point, and (ii) inputting the corresponding genotypic data construct into the model (e.g., the same disease classification model 142 as used to generate disease class model score sets 146 for the test subject), to generate a corresponding reference time stamped model score set for the disease condition at the respective time point for the corresponding reference subject. Thereby, a corresponding plurality of reference time stamped model score sets for the corresponding reference subject can be formed, where each respective reference time stamped model score set for a different time point in the corresponding plurality of time points associated with the corresponding reference subject. The corresponding plurality of referenced time stamped time model score sets can then be fitted with the temporal trend test (e.g., the same temporal trend test used to fit the disease class model score sets 146 of the test subject), thereby obtaining the respective trend parameter in the distribution of trend parameters for the corresponding reference subject.

Some aspects of the present disclosure can be based on, at least in part, the recognition that accounting for personal characteristics of the test subject can improve the sensitivity and specificity of methods for classifying a disease state in the test subject. That is, because personal characteristics of the test subject can affect the manifestation of the disease state biological signature of the test subject. As such, accounting for one or more of these personal characteristics of the test subject can further improve the sensitivity and specificity of the disease state classification. For instance, the magnitude of a change between consecutive disease class model score sets in a series of disease class model score sets, as well as the significance of the change, are affected by at least (i) changes in the disease state of the test subject, e.g., development and progression of the disease state can increase the magnitude of the disease class model score set while regression of the disease state can decrease the magnitude of the disease class model score set, (ii) background variance in the biological characteristics that constitute the disease state signature of the subject, (iii) personal characteristics of the test subject, e.g., age, gender, ethnicity, smoking status, alcohol consumption, familial history, etc., and (iv) the length of time between consecutive time points. For example, a 10 percent increase in the probability the subject has a particular disease state is less significant if the length of time between sample collection events is twenty years than if the time between sample collection events is two months.

Accordingly, in some embodiments, one or more of factors affecting the magnitude and/or significance of the change between consecutive disease class model score sets in a time series of disease class model score sets are accounted for when evaluating the temporal trend test parameter for the test subject against the distribution of reference trend test parameters. In some embodiments, these features are accounted for by adjusting or normalizing either, or both, of the trend test parameter and the distribution of reference trend test parameters. In some embodiments, the adjustment or normalization is applied to the trend test parameter and/or the reference trend test parameters directly, e.g., each trend test parameter is adjusted or normalized independent of each other. In some embodiments, adjustment or normalization is applied to the reference trend test parameters through the reference distribution, e.g., individual reference trend test parameters are adjusted or normalized as a function of the distribution, rather than on an individualized basis. In some embodiments, the underlying biological feature data, which is evaluated by the disease classification model, is adjusted or normalized.

In some embodiments, the length of time between collection of consecutive biological samples from the test subject and/or reference subject, e.g., an average length of time between collection of all the biological samples in the time series, is used for adjustment or normalization, e.g., the test subject and/or reference subject biological data, and/or the test subject and/or reference subject trend test parameters, and/or the distribution of reference trend test parameters are adjusted or normalized to account for the time between biological sample collections.

Accordingly, in some embodiments, an amount of time between consecutive time points (e.g., an average length of time between biological sample collections in the time series) for each respective reference subject in the plurality of reference subjects is used as a covariate in calculating the distribution (e.g., the distribution of reference trend test parameters). The trend test parameter (e.g., trend test parameter 149) can then be adjusted based on the covariate representing a difference in time between consecutive test time points (e.g., an average length of time between biological sample collections from the test subject in the time series). In some embodiments, the covariate representing a difference in time between consecutive test time points is applied to one or more genotypic characteristics in the plurality of characteristics of either or both of the genotypic data constructs (e.g., genotypic data constructs 142) corresponding to the consecutive time points, for either or both of the test subject or the reference subjects. In some embodiments, the covariate representing a difference in time between consecutive time points in a time series is applied to the trend test parameter (e.g., trend test parameter 149) and each reference trend test parameter in the distribution of trend test parameters.

Similarly, in some embodiments, each respective trend test parameter in the plurality of reference trend test parameters is normalized for an amount of time between consecutive time points in a time series for the respective subject, and the test trend test parameter is normalized for an amount of time between consecutive time points in a time series for the test subject. Likewise, in some embodiments, each respective reference trend test parameter in the plurality of reference trend test parameters is normalized for an amount of time between consecutive time points in a time series for the respective reference subject by normalizing one or more genotypic characteristics in the plurality of characteristics of either or both of the respective reference genotypic data construct corresponding to the consecutive time points in the time series for the respective subject. The test trend test parameter can be normalized for an amount of time between consecutive test time points in the time series for the test subject by normalizing one or more genotypic characteristics in either or both of the genotypic data constructs corresponding to the consecutive time points in the time series for the test subject. In some embodiments, the normalizing is applied to the test trend test parameter and each reference trend test parameter in the distribution of the reference trend test parameters.

In some embodiments, the age of the test and/or reference subject is used for adjustment or normalization, e.g., the test subject and/or reference subject biological data, and/or the test subject and/or reference subject trend test parameters, and/or the distribution of reference trend test parameters are adjusted or normalized to account for the age of the test subject.

Accordingly, in some embodiments, an age of each respective reference subject in the plurality of reference subjects is used as a covariate (462) in calculating the distribution (e.g., the distribution of reference trend test parameters). The test trend test parameter (e.g., trend test parameter 149) can then be adjusted based on an age of the test subject. In some embodiments, the covariate representing the age of the test subject is applied to one or more genotypic characteristics in the plurality of characteristics of one or more genotypic data construct (e.g., genotypic data construct 142) in the plurality of genotypic data constructs for the test subject, and/or for one or more genotypic data construct in the plurality of genotypic data constructs for each respective reference subject in the plurality of reference subjects. In some embodiments, the covariate representing the age of the test subject is applied to the test trend test parameter (e.g., trend test parameter 149) and each reference trend test parameter in the distribution of reference trend test parameters.

Similarly, in some embodiments, each respective reference trend test parameter in the plurality of reference trend test parameters is normalized for an age of the respective reference subject, and the test trend test parameter is normalized for an age of the test subject. Each respective reference trend test parameter in the plurality of reference trend test parameters can be normalized for an age of the respective reference subject by normalizing one or more genotypic characteristics in the plurality of characteristics of each respective reference genotypic data construct for the age of the respective subject, and the test trend test parameter is normalized for age of the test subject. In some embodiments, the normalizing is applied to the test trend test parameter and each reference trend test parameter in the distribution of the reference trend test parameters.

In some embodiments, the smoking status or an alcohol consumption characteristic of the test and/or reference subject is used for adjustment or normalization, e.g., the test subject and/or reference subject biological data, and/or the test subject and/or reference subject trend test parameters, and/or the distribution of reference trend test parameters are adjusted or normalized to account for the smoking status or an alcohol consumption characteristic of the test subject.

Accordingly, in some embodiments, a smoking status or an alcohol consumption characteristic of each respective reference subject in the plurality of reference subjects is used as a covariate (464) in calculating the distribution (e.g., the distribution of reference trend test parameters). The test trend test parameter (e.g., trend test parameter 149) can then be adjusted based on a smoking status or an alcohol consumption characteristic of the test subject. In some embodiments, the covariate representing the smoking status or an alcohol consumption characteristic of the test subject is applied to one or more genotypic characteristics in the plurality of characteristics of one or more genotypic data construct (e.g., genotypic data construct 142) in the plurality of genotypic data constructs for the test subject, and/or for one or more genotypic data construct in the plurality of genotypic data constructs for each respective reference subject in the plurality of reference subjects. In some embodiments, the covariate representing the smoking status or an alcohol consumption characteristic of the test subject is applied to the test trend test parameter (e.g., trend test parameter 149) and each reference trend test parameter in the distribution of reference trend test parameters.

Similarly, in some embodiments, each respective reference trend test parameter in the plurality of reference trend test parameters is normalized for a smoking status or an alcohol consumption characteristic of the respective reference subject, and the test trend test parameter is normalized for a smoking status or an alcohol consumption characteristic of the test subject. Each respective reference trend test parameter in the plurality of reference trend test parameters can be normalized for a smoking status or an alcohol consumption characteristic of the respective reference subject by normalizing one or more genotypic characteristics in the plurality of characteristics of each respective reference genotypic data construct for the smoking status or an alcohol consumption characteristic of the respective subject, and the test trend test parameter is normalized for the smoking status or an alcohol consumption characteristic of the test subject. In some embodiments, the normalizing is applied to the test trend test parameter and each reference trend test parameter in the distribution of the reference trend test parameters.

In some embodiments, the gender of the test and/or reference subject is used for adjustment or normalization, e.g., the test subject and/or reference subject biological data, and/or the test subject and/or reference subject trend test parameters, and/or the distribution of reference trend test parameters are adjusted or normalized to account for the gender of the test subject.

Accordingly, in some embodiments, a gender/biological sex of each respective reference subject in the plurality of reference subjects is used as a covariate (466) in calculating the distribution (e.g., the distribution of reference trend test parameters). The test trend test parameter (e.g., trend test parameter 149) can then be adjusted based on a gender of the test subject. In some embodiments, the covariate representing the gender of the test subject is applied to one or more genotypic characteristics in the plurality of characteristics of one or more genotypic data construct (e.g., genotypic data construct 142) in the plurality of genotypic data constructs for the test subject, and/or for one or more genotypic data construct in the plurality of genotypic data constructs for each respective reference subject in the plurality of reference subjects. In some embodiments, the covariate representing the gender of the test subject is applied to the test trend test parameter (e.g., trend test parameter 149) and each reference trend test parameter in the distribution of reference trend test parameters.

Similarly, in some embodiments, each respective reference trend test parameter in the plurality of reference trend test parameters is normalized for a gender of the respective reference subject, and the test trend test parameter is normalized for a gender of the test subject. Each respective reference trend test parameter in the plurality of reference trend test parameters can be normalized for a gender of the respective reference subject by normalizing one or more genotypic characteristics in the plurality of characteristics of each respective reference genotypic data construct for the gender of the respective subject, and the test trend test parameter is normalized for the gender of the test subject. In some embodiments, the normalizing is applied to the test trend test parameter and each reference trend test parameter in the distribution of the reference trend test parameters.

In some embodiments, rather than adjusting or normalizing a single distribution of trend test parameters, a segmented reference distribution is used in which all of the reference subjects are one of an enumerated class of individuals sharing one or more personal characteristics with the test subject. For example, in some embodiments, a reference distribution is selected such that all of the reference subjects used in the reference distribution have a similar age as the test subject. In some embodiments, system 100 stores a plurality of segmented reference distributions, or forms a segmented reference distribution based on one or more personal attributes of the test subject. In some embodiments, each reference subject in a segmented distribution has an age, gender, smoking status, and/or alcohol consumption characteristic that is shared with the test subject. Accordingly, in some embodiments, the plurality of reference subjects is segmented for gender, age, smoking status, alcohol consumption, background variance in a biological characteristic, or a combination thereof (468). Such segmented distribution can include information about dependency structure among different covariates. For instance, a segmented reference distribution is formed from trend test parameters that share one or more enumerated personal characteristic with the test subject. In one example, a segmented reference distribution can be formed from trend test parameters that share the same gender, age, and smoking status.

In some embodiments, the test trend test parameter (e.g., trend test parameter 149) is evaluated by performing a statistical hypothesis test against a reference distribution of trend test parameters from reference subjects that are not afflicted with the disease state, which may or may not be adjusted or normalized to account for a covariate. In some embodiments, the statistical hypothesis test provides a measure of statistical significance for whether or not the test trend test parameter is a member of the distribution of reference trend test parameters. In some embodiments, the subject is deemed to be afflicted with the disease state when the statistical hypothesis test provides a p-value that satisfies a threshold level of significance, e.g., p=0.05, 0.1, 0.005, etc.

However, because p-values measure the aggregated probability that a defined event (e.g., the null hypothesis), or an occurrence more rare than the defined event, a statistically significant p-value cannot identify whether the defined event falls on one extreme or the other extreme within the distribution. Accordingly, in some embodiments, comparison of the test trend test parameter and the distribution of reference trend test parameters further uses inspection as to which extreme the test trend test parameter belongs. For instance, negative changes in the disease class model score set can indicate that the disease is regressing in the subject, rather than progressing.

In some embodiments, the comparison between the test trend test parameter and the distribution of reference trend test parameters includes determining (456) a measure of central tendency of the distribution and a measure of spread of the distribution. Then, the comparison can include determining a significance of the test trend test parameter using the measure of central tendency of the distribution and the measure of spread of the distribution. In some embodiments, the measure of central tendency of the distribution is an arithmetic mean, weighted mean, midrange, midhinge, trimean, Winsorized mean, mean, or mode across the distribution. In some embodiments, the measure of spread of the distribution is a standard deviation, a variance, or a range of the distribution.

In some embodiments, the measure of central tendency of the distribution is the mean of the distribution, the measure of spread of the distribution is the standard deviation of the distribution, and the determining the significance of the test trend test parameter using the measure of central tendency of the distribution and the measure of spread of the distribution comprises determining a number of standard deviations the test trend test parameter is from the mean of the distribution (458). In some embodiments, the test subject is determined to have the disease condition when the number of standard deviations the test trend test parameter from the mean of the distribution satisfies a threshold value (460). That is, it can be expected that the test subject does not have the disease condition (e.g., cancer or coronary disease condition) if their trend test parameter is similar to those in the distribution.

In some embodiments, the test trend test parameter is evaluated by logistic regression, rather than statistics. For instance, in some embodiments, the evaluating includes evaluating the test trend test parameter using a logistic function trained by logistic regression against the plurality of reference trend test parameters. In some embodiments, each reference trend parameter set in the plurality of reference trend parameter sets is for a respective reference subject in the plurality of reference subjects based on a difference between (i) a first time stamped model score set provided by the embedding layer of the model using a first respective reference genotypic data construct comprising values for the plurality of genotypic features, taken using a first respective biological sample acquired at a respective first time point from the respective reference subject, and (ii) a second time stamped model score set provided by the embedding layer of the model using a second respective genotypic data construct comprising values for the plurality of genotypic features, taken using a second respective biological sample acquired from the respective reference subject at a respective second time point other than the first respective time point.

In some embodiments, the logistic regression further includes personal characteristics, for example one or more of gender, age, smoking status, and alcohol consumption, in order to account for such characteristics, as described above for the statistical methods.

The regression algorithm can be any type of regression. For example, in some embodiments, the regression algorithm is logistic regression. In some embodiments, the logistic regression assumes:

$$P ( {x}_{i} ) = \frac{exp ( {β}_{0} + {β}_{1} {x}_{i 1} + … + {β}_{k} {x}_{ik} )}{1 + exp ( {β}_{0} + {β}_{1} {x}_{i 1} + … + {β}_{k} {x}_{ik} )} ,$$

where:
   xᵢ=(xᵢ₁, xᵢ₂, . . . , xᵢₖ) are the corresponding biological feature values (e.g., one or more of read counts 126, allele statuses 130, allelic fractions 134, and methylation statuses 138), obtained from biological samples for the i^{th} corresponding training subject, where the i^{th} corresponding training subject either has a first disease status (e.g., cancer condition or coronary disease) (Y=1) or a second disease status (Y=0);
   Y∈{0, 1} is a class label that has the value “1” when the corresponding subject i has the first disease status and has the value “0” when the corresponding subject i has the second disease status,
   β₀ is an intercept, and
   βⱼ=(j=1, . . . k) is a plurality of regression coefficients, where each respective regression coefficient in the plurality of regression coefficients is for a corresponding biological feature value.

In some embodiments, the logistic regression is logistic least absolute shrinkage and selection operator (LASSO) regression. In some such embodiments, the logistic LASSO estimator [symbol] , . . . , [symbol] is defined as the minimizer of the negative log likelihood:

min(Σᵢ₌₁ⁿ[−yᵢ(β₀+β₁xᵢ+ . . . +βₖxᵢₖ)+log(1+exp(β₀+β₁xᵢ+ . . . +βₖxᵢₖ))]),

subject to the constraint Σⱼ₌₁^{k}|βⱼ|≤λ, where λ is a constant optimized for any given dataset.

In some embodiments, the regression algorithm is logistic regression with lasso, L2 or elastic net regularization.

As noted in the above equations, each xᵢ=(xᵢ₁, xᵢ₂, . . . , xᵢₖ) are the corresponding feature values for the i^{th} corresponding training subject and, as such, each xᵢ, represents a corresponding biological feature. Moreover, each βⱼ=(j=1, . . . k) is the regression coefficient for a corresponding biological feature. In some embodiments, those extracted features that have a corresponding regression coefficient that fails to satisfy a threshold value are pruned (removed from) the plurality of biological features. In some embodiments, this threshold value is zero. Thus, in such embodiments, those biological features that have a corresponding regression coefficient that is zero from the above-described regression are removed from the plurality of biological features prior to training the classifier. In some embodiments, for instance, in which L2 regularization is employed, the threshold value is 0.1. Thus, in such embodiments, those biological features that have a corresponding regression coefficient whose absolute value is less than 0.1 from the above-described regression are removed from the plurality of extracted features prior to training the classifier. In some embodiments, the threshold value is a value between 0.1 and 0.3. An example of such embodiments is the case where the threshold value is 0.2. In such embodiments, those extracted features that have a corresponding regression coefficient whose absolute value is less than 0.2 from the above-described regression are removed from the plurality of extracted features prior to training the classifier.

### Examples

The data used in the analyses presented in Examples 1 and 2 below was collected as part of the CCGA clinical study. The CCGA [NCT02889978] is the largest study of cfDNA-based early cancer detection. This prospective, multi-center, observational study has enrolled over 10,000 demographically-balanced participants across 141 sites, including healthy individuals and cancer patients across at least 20 tumor types and all clinical stages. All samples were analyzed by: 1) Paired cfDNA and white blood cell (WBC)-targeted sequencing (60,000×, 507 gene panel), using a joint caller to remove WBC-derived somatic variants and residual technical noise; 2) Paired cfDNA and WBC whole-genome sequencing (WGS) at approximately 35×sequence coverage; and 3) cfDNA whole-genome bisulfite sequencing (WGBS) at approximately 34×sequence coverage, using abnormally methylated fragments to normalize scores.

Cell-free DNA was isolated from the collected blood samples and then sequenced, as described above, to provide the cfDNA sequencing data. Likewise, blood cells were isolated using a buffy coat separation method and genomic preparations from the white blood cells were then sequenced to provide a matching sequence reads of the loci of interest, e.g., for positive assignment of sequence variants arising from clonal hematopoiesis.

The cancer types included in the CCGA study included invasive breast cancer, lung cancer, colorectal cancer, DCIS, ovarian cancer, uterine cancer, melanoma, renal cancer, pancreatic cancer, thyroid cancer, gastric cancer, hepatobiliary cancer, esophageal cancer, prostate cancer, lymphoma, leukemia, multiple myeloma, head and neck cancer, and bladder cancer.

### Example 1—In Silico Spiking of Cancer Signals into Data from Non-cancerous Subjects

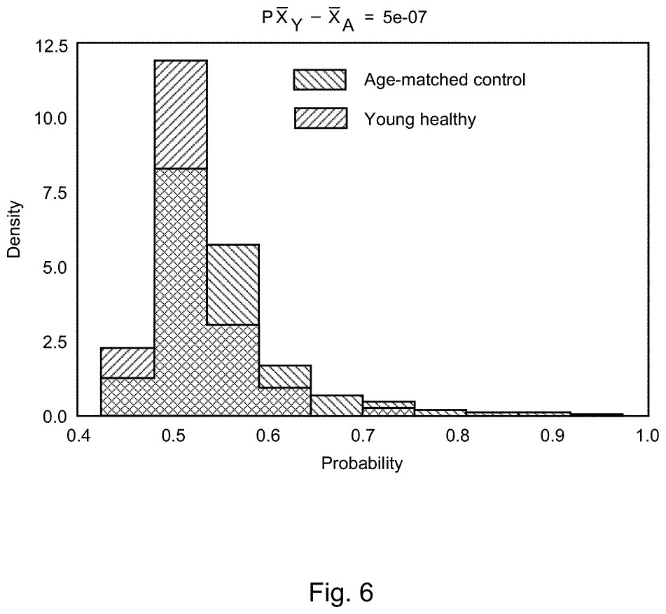

It was hypothesized that pre-cancerous genomic aberration accumulates with age, but is held in check by the immune system, telomeric shortening, etc., until appropriate (and evolutionarily unlikely) adaptations arise. That is, cancer evolution becomes punctuated/saltational at evolutionary bottlenecks. That is development of a biological signature for cancer in a subject developing cancer (e.g., having progressing, early-stage cancer) would proceed differently in different subjects, due to biological differences between the subjects, e.g., aging. For example, FIG. 6 shows two distributions of cancer model probabilities calculated for healthy individuals based on inspection of cfDNA sequence reads. Distribution XA included non-cancer patients from the CCGA control group matched in age distribution to the CCGA cancer patients. Distribution XB included young and healthy individuals from the CCGA control group. As shown in FIG. 6, there was a statistically significant difference between the two distributions (p=0.0000005). This reinforces the conclusion that age plays a key role in the development of cancer signal. Therefore, adjusting for this variation through the use of personalized baselines for biological features could improve the level of detection of any cancer classifier.

To investigate this theory, an in silico data spiking experiment was designed to test the effect of spiking the same amount of various cancer signals into different biological backgrounds. In the experiment, increasing percentages of bin values determined for sequence reads mapped to a plurality of genomic regions from subjects known to have various types of cancer were serially spiked into bin values determined for sequence reads mapped to the plurality of genomic regions for subjects with very low tumor fractions. This was designed to simulate a time series development of cancer, in silico, using a plurality of different biological backgrounds. Then, development of the cancer signal, as reported by a probability of cancer derived from a cancer classifier trained against copy number variation (relative bin values), was evaluated for each spiked data sample. The classifier used in this experiment is described in U.S. Patent Application Publication No. 2019/0287649.

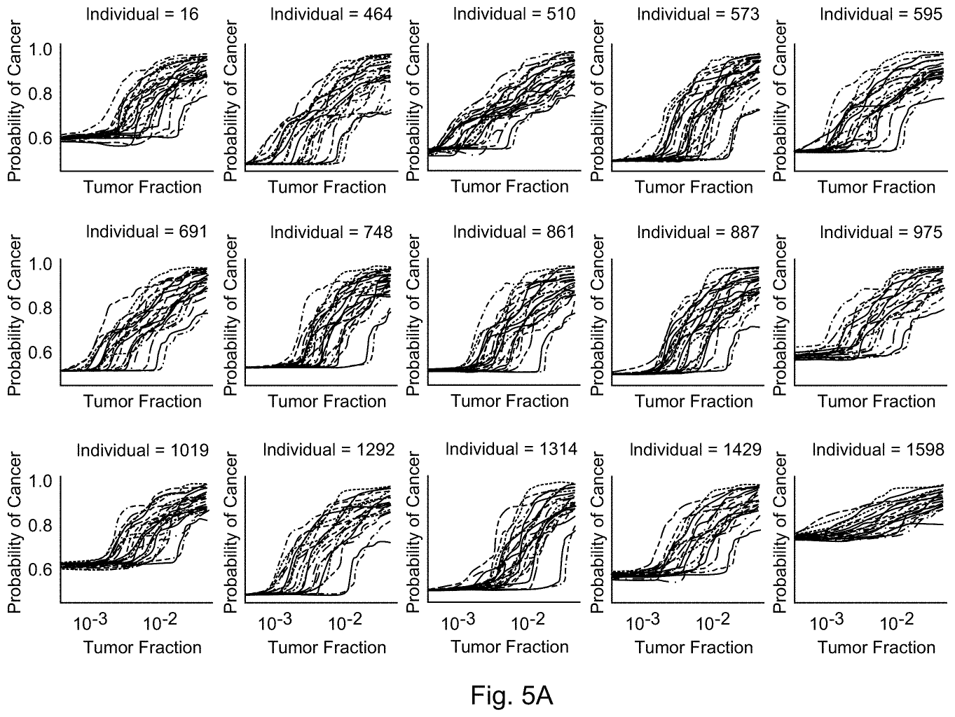

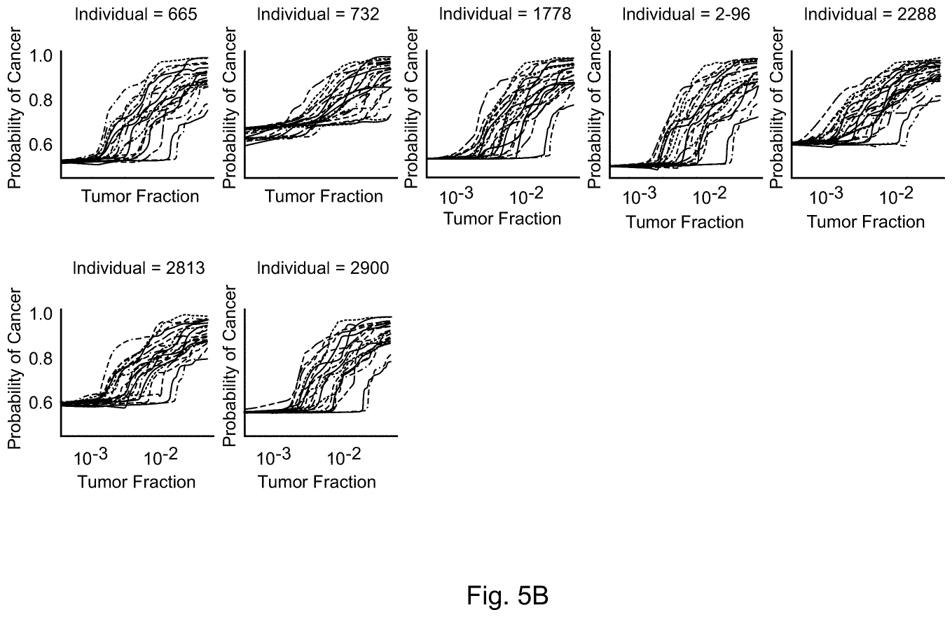

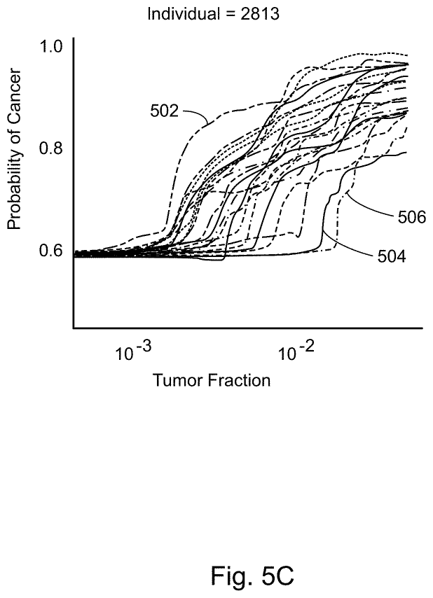

Briefly, twenty-two CCGA low-tumor-fraction subjects with undetectable levels of cell-free tumor fraction, and a matched number of high-tumor-fraction subjects who were known to have different types of cancer, who each had a cell-free DNA tumor fraction of at least 10%, and for whom the cancer classifier provide at least a 90% probability of having cancer, were also selected from the CCGA study data. Next, increasing amounts of bin counts from each of the high-tumor-fraction subjects were added to the bin counts of different instances of the bin count data for each low-tumor-fraction subject, forming four hundred and eighty four sets of cancer series data having increasing bin counts, as plotted on the x-axis of the graphs shown in FIG. 5. Each instance of spiked bin counts was then evaluated by the cancer classifier, to generate a probability that the spiked data was acquired from a subject having cancer. These probabilities were plotted as a function of tumor fraction, in the graphs shown in FIG. 5.

As shown by the graphs in FIG. 5, the probability of cancer calculated for a given simulated sample depended upon (i) the simulated tumor fraction, (ii) the type of cancer, and (iii) the background signal provided by the reference subject (the subject who data was spiked with cancer signal). For instance, referring to reference individual 2813, the plot for which is enlarged in FIG. 5C, there is a nearly 10-fold difference in the tumor fraction used to generate a spike in the identified cancer probability across the different types of cancers. For instance, when signal from a first cancer was spiked into reference individual's 2813 background (represented by series 502), a significant increase in the identified cancer probability was seen at simulated tumor fractions of just greater than 0.001 (0.1%). However, when signal from two different cancers were spiked into the same background (represented by series 504 and 506, respectively), an increase in the identified cancer probability was not seen until the simulated tumor fraction increases above 0.01 (1%). This demonstrates the dependence upon the cancer type on the calculated cancer probability. Similarly, FIG. 5 shows that the dependence upon the individual's background signal on the calculated cancer probability is rather significant. For instance, in most of the reference backgrounds, a spike in calculated cancer probability was not observed for one particular cancer type until the tumor fraction of the simulated sample reached above 0.01 (1%). However, when the cancer signal for that cancer was spiked into data for individual 510, a spike in cancer probability was observed at a tumor fraction significantly below 0.01. In fact, detectable spikes in the calculated cancer probabilities for reference individual 510 were seen significantly earlier for almost all of the different cancer types. In contrast, when the cancer signal for that cancer type was spiked into data for individual 1314, no increase in cancer probability was observed until the tumor fraction rose significantly above 0.01 (1%). In fact, detectable spikes in the calculated cancer probabilities for reference individual 1314 appeared to be significantly delayed for most cancer types.

### Example 2—Testing of In Silico Distributions

The in silico time series data generated for the sample of cancer types spiked into 22 different reference backgrounds, described in Example 1, was used as data set to test whether the methods described herein for comparing changes in cancer probability over time to a reference distribution can increase the sensitivity of a classifier for cancer. Two different approaches were taken to generate a reference distribution to which the changes in cancer probabilities shown in FIG. 5 could be compared.

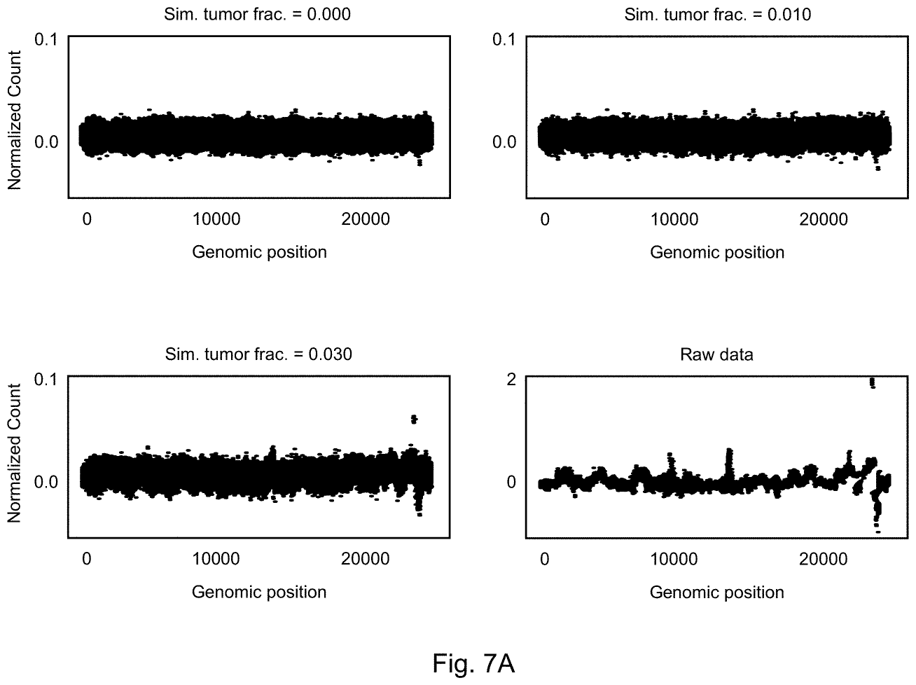

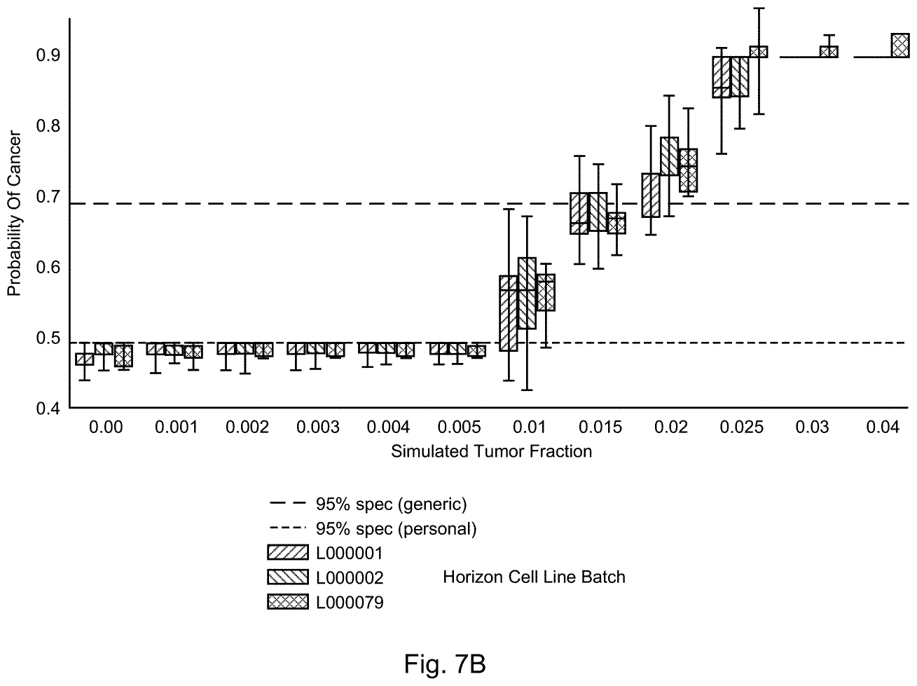

In a first approach, bin counts were determined for more than 100 samples of a single positive cancer cell line control. As these samples contained cancerous cells, the effective tumor fraction for the sample was known to be 1.0. Given data from a reference, non-cancerous sample, having an effective tumor fraction of 0.0, regression analysis was used to simulate signals from a plurality of tumor fractions between 0.0 and 1.0, as shown in FIG. 7A. Cancer probabilities for each regressed tumor fraction, for each reference sample were then generated using the copy number classifier described in U.S. Patent Application Publication No. 2019/0287649. Examples of the calculated cancer probabilities generated for three of the simulated tumor fraction series are illustrated in FIG. 7B.

Next, a distribution of changes in the probability of cancer as a function of tumor fraction was established based on the regressions performed for all samples. The distribution was defined to include those healthy samples with no spiked in cancer DNA signal. Then, the changes in cancer probability for all samples was compared to the established reference distribution. As shown in FIG. 7B, when the copy number classifier was used alone to classify whether the samples were cancerous, 95% specificity was reached at a tumor fraction of approximately 0.02 (2%). However, when the changes in probability between consecutively simulated data set was compared to the established baseline, using a 95% statistical cut-off (p=0.05), 95% specificity was achieved at a tumor fraction of approximately 0.01, representing a 2-fold improvement in LoD, the tumor fraction at which 50% sensitivity was achieved.

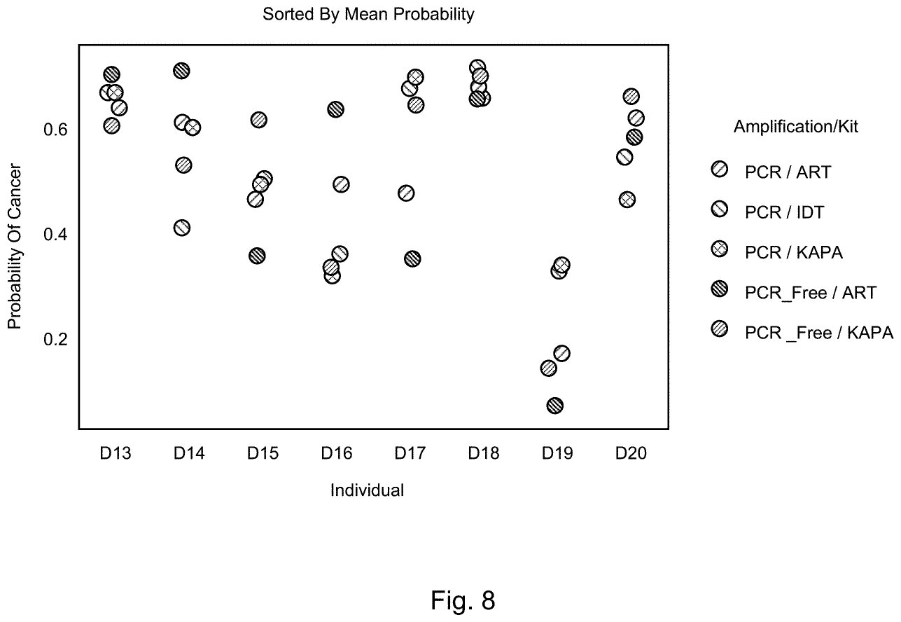

In a second approach, three replicates of samples from eight different healthy individuals, using five different combinations of cfDNA isolation and amplification protocols, were used to establish a normalized distribution of cancer probabilities for intra-individual variance, as illustrated in FIG. 8. 95% specificity was achieved at a tumor fraction of approximately 0.08 (8%) using this distribution.

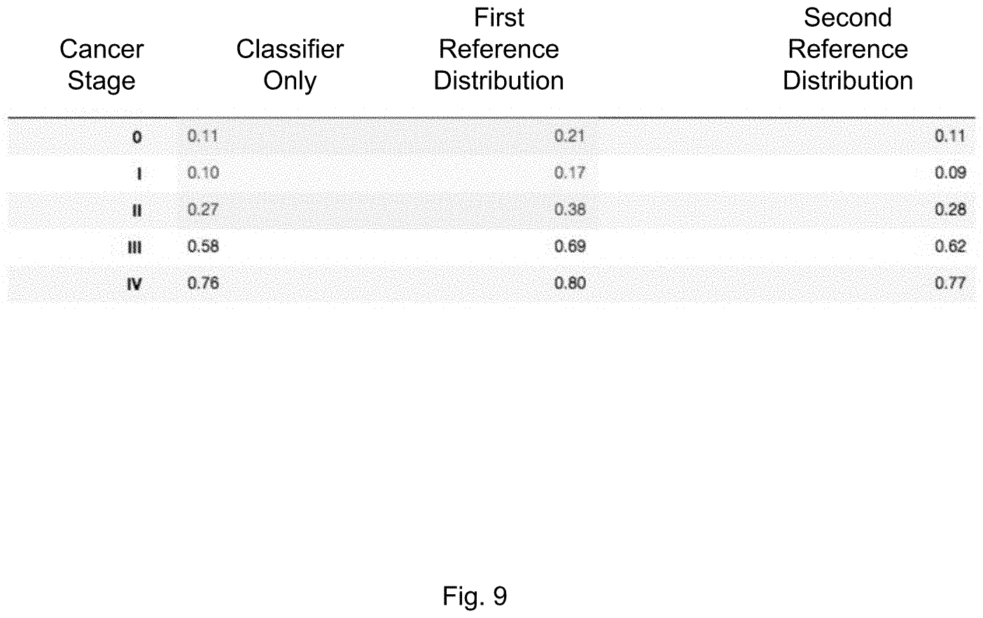

Next, the two distributions established above, were used for comparison of changes in the cancer probabilities for all of the simulated tumor fraction series data described in Example 1. A 95% statistical cut-off (p=0.05), was used to call whether the sample can be classified as cancerous or non-cancerous. FIG. 9 shows a breakdown of the sensitivity of the various models achieved for each cancer stage, as defined by simulated tumor fraction. Briefly, the data shows that using the first reference distribution, the comparative change in cancer method described herein approximately doubled the sensitivity at 95% specificity for detecting stage 0 cancer, improved the sensitivity for detecting stage I cancer by approximately 70%, improved the sensitivity for detecting stage II cancer by approximately 40%, and improved the sensitivity for detecting stage III cancer by approximately 20%. Advantageously, these improvements in sensitivity would significantly improve detection of early stage cancers, as compared to convention, single-time point assays.

### Example 3—CCGA Serial Sample Study—Sub Study

A study was developed to determine whether changes in patient results over time from a next generation sequencing (NGS)-based cancer classifier, developed and validated in a separate study (CCGA), could be used to identify early stage cancer in subjects classified as non-cancerous by the classifier. Briefly, cell-free DNA (cfDNA) isolated from plasma collected from subjects was sequenced and analyzed using a classifier trained to distinguish between multiple types of cancer and to provide cancer tissue of origin information. The output of the test provided a diagnosis or prediction selected from a group of diagnoses that includes at least (i) no cancer signal detected, indicating the subject does not have cancer, (ii) a cancer signal with an indeterminate tissue of origin, indicating the subject has cancer originating from an undetermined tissue type, and (iii) a cancer signal with a determined tissue of origin, indicating the subject has cancer originating from a particular tissue type.

The objectives of the study were: (i) to evaluate cfDNA signatures in individuals serially over time, (ii) to describe the association between changes in cfDNA signatures over time and cancer diagnoses, and (iii) to describe the association between changes in cfDNA signatures over time and subject outcomes. Accordingly, the overall goal of the study was to explore changing cancer signals over time and demonstrate increased cancer detection sensitivity and specificity, when serial blood draws are available.

This study is a sub-study of the CCGA. The CCGA is a prospective, multi-center, observational study with collection of de-identified biospecimens and clinical data from at least 15,000 participants from clinical networks in the United States, Canada, and the United Kingdom. The study enrolled cancer subjects with multiple types of malignancies (the CANCER arm) and representative subjects without a clinical diagnosis of cancer (the NON-CANCER arm) as defined by eligibility criteria over an enrollment period of 30 months. Clinical information, demographics, and medical data relevant to cancer status were collected from all participants and their medical record at baseline (time of biospecimen collection), and subsequently from the medical record at intermittent future time points, at least annually for up to 5 years. A future blood collection may also be requested from study subjects during the follow-up period, but is not a scheduled event.

The Sub-Study population is derived from the enrolled CCGA population. Current CCGA participants were selected for inclusion in the Sub-Study as defined by eligibility criteria. Subjects agreeing to participate underwent an enrollment Study Visit for consent. Consenting subjects underwent two study blood draws approximately 3 months apart. Additional clinical information regarding past and current health status was collected. This included but were not limited to past medical history, current medical conditions, diagnostic and screening tests, and health-related risk factors. 400 participants were enrolled for the Sub Study, 200 with a diagnosis of cancer in the enrollment period and 200 with no cancer diagnosis in the enrollment period. Sub Study participation included 2 additional blood draws 3 months apart and follow-up within the protocol defined CCGA study period, which is up to 5 years following enrollment. Participation in the Sub Study did not extend the study duration beyond that already prescribed in CCGA protocol.

Briefly, venous blood was collected from the Sub Study participants by peripheral venous blood draw with optimal collection of 20 mL (maximum) peripheral blood into 2×10 mL Streck Cell-free DNA BCT. In addition, clinical data was collected from participant questionnaires and the medical record (at baseline and follow-up visits), including imaging and pathology reports. Data was captured and managed within an electronic data capture (EDC) system.

### Example 4—Temporal Methylation Changes

A study was performed to evaluate changes in genomic methylation patterns over time and, particularly, changes in genomic methylation patterns that indicate pre-cancer and/or early cancer development. This study was a sub-study of the CCGA. To date CCGA-based studies have evaluated blood draws from a single point in time from a given donor. Though useful for identifying dominant methylation variants present in cancer patients versus normal participants, single time point observations do not assess participant-level epigenetic changes that occur with time in non-cancer participants.

As a first objective of the study, temporal methylation changes in healthy participants were investigated. Briefly, follow-up blood draws were collected from selected CCGA2 participants for processing with a targeted methylation assay. Longitudinal velocity of methylation patterns were characterized from a comparison of the methylation patterns in the original CCGA2 blood samples to those subsequent blood draws. The results from this first objective were used to design follow-up studies to address secondary research objectives. These secondary objectives include (i) improving classifier performance using longitudinal blood draws, (ii) identifying temporal changes in methylation pattern that accompany and/or drive transformation from a non-cancerous state to a cancerous state in a subject, (iii) assessing the velocity of epigenetic changes in a cancer signal over time, and (iv) evaluating whether particular individuals have inherently noisy methylation signals that persist in repeated blood draws.

Briefly, 188 CCGA2 participants with longitudinal blood draws were selected for this study. These CCGA2 participants had an evaluable assay result at baseline and an additional blood draw later in time. A single tube of plasma from each participant was selected for processing. Participants were selected or prioritized based on the following criteria: (i) the subject had strong cancer signal at the time of the first blood draw, as determined by a positive cancer prediction from the multi-cancer classifier at a specificity of 97%, 98%, and 99%; (ii) that DNA sequencing data from corresponding white blood cells from the subject was available; (iii) that the selected cohort have a roughly uniform distribution of subjects having longitudinal samples collected around 12 months, 18 months, 24 months, and 30 months after the baseline blood draw; (iv) that the selected cohort have approximately the same number of males and females; and (v) that the selected cohort have a roughly equal number of participants from each of the following age groups: <30, 31-40, 41-50, 51-60, 61-70, 71-80, and >80.

188 frozen longitudinal CCGA plasma samples were processed, and two cfDNA extraction batches (plates) were processed and quantified. 2 PC2 positive controls, representing control samples formulated to provide abnormal counts upon processing in a multi-cancer assay, were added to each plate of samples at the cfDNA extraction step. The samples have been formulated to provide consistent abnormal and binary coverage in a multi-cancer assay and serve as experimental quality controls. The samples in the two plates were subject to bisulfite conversion, DNA library preparation, and sample quantification. Finished cfDNA libraries were quantified with Accuclear and consolidated for multiplex enrichment. A multiplex enrichment protocol using a probe library that enriches for CpG-rich regions, library quantification, and normalized pooling was performed, e.g., as described in United States Patent Publication No. US 2020-0365229 A1. All samples were then sequenced on a single S4 flow cell.

The sequencing data was de-multiplexed and input into a cfDNA methylation-based multi-cancer classifier, e.g., as described in United States Patent Publication No. US 2020-0365229 A1, which is hereby incorporated by reference, implemented at a target specificity of 99.4%. Two versions of the assay (Methylation Test v1 and Methylation Test v2) were used in the study, based on which assay was originally used to evaluate the first blood draw from the subject in the CCGA2 study data.

The classifier outputs a probability score, ranging from 0 to 1, representing the cancer signal at the time of the corresponding blood draw. Statistical analyses on the change in the output score generated for each subject between the initial and longitudinal sample blood draw (e.g., second blood draw) were then evaluated for qualitative insights into the key objectives described above.

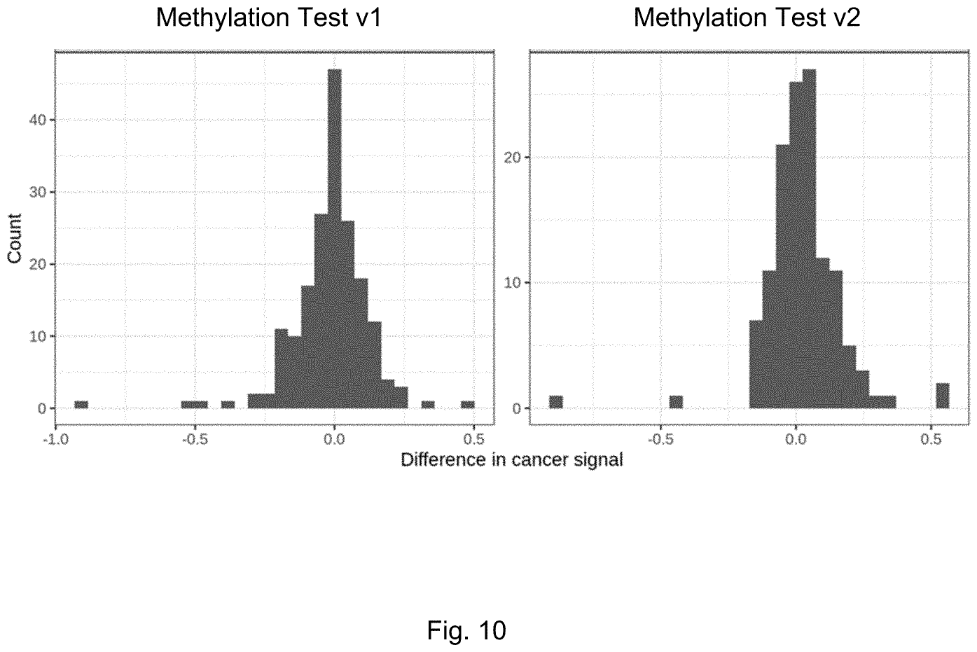

First, the distribution of changes in the probability score generated for each subject between the first and second samples were determined. Histograms of these changes are presented in FIG. 10, for samples processed using version 1 (left) and version 2 (right) of the methylation assay at the initial blood draw. As can be seen in FIG. 10, the distribution of changes clustered around 0 for both versions of the assay. Further, the distribution appeared to be fairly regular, with similar numbers of changes greater than and less than zero. This likely represents background variance in the methylation signals of these healthy subjects. That is, fluctuations in the genomic methylation pattern over the 12 to 40 month period, for the most part, result in small shifts in the cancer probability output by the classifier.

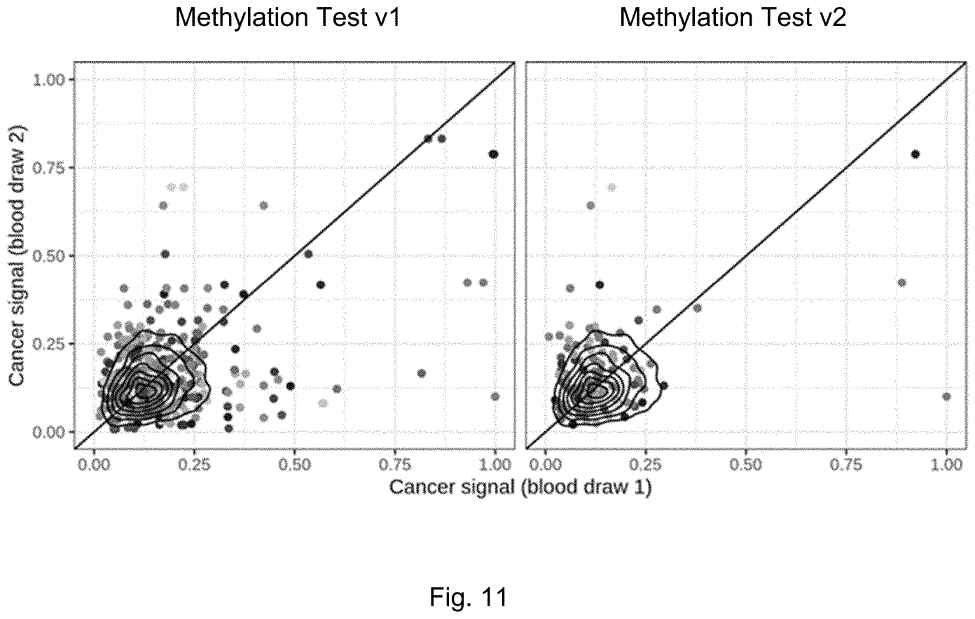

Next, the second cancer probability score generated for each subject (using the second, longitudinal blood draw) was plotted as a function of the first cancer probability score for the subject (using the first blood draw). As shown in FIG. 11, the majority of points fell in the lower left quadrant of the plot, representing cases where the cancer probability score generated from both the first and second blood draw were low. In a few instances, the points fell in the upper right quadrant of the plot, representing cases where the cancer probability score generated from both the first and second blood draw were high. However, in a few instances, significant changes in the cancer probability score were observed, represented by the points falling within the upper left and lower right quadrants of the graph. For perspective, a density plot representing variation in cancer probability score between v1 assay replicates from 4503 CCGA2 participants, is overlaid, in unbroken lines, on the plot. Significantly, the majority of points, particularly when version 2 of the methylation test was used at the initial blood draw, fall within this distribution, indicating that some of the small changes in cancer probability score can be attributed to noise within the assay, rather than underlying biology.

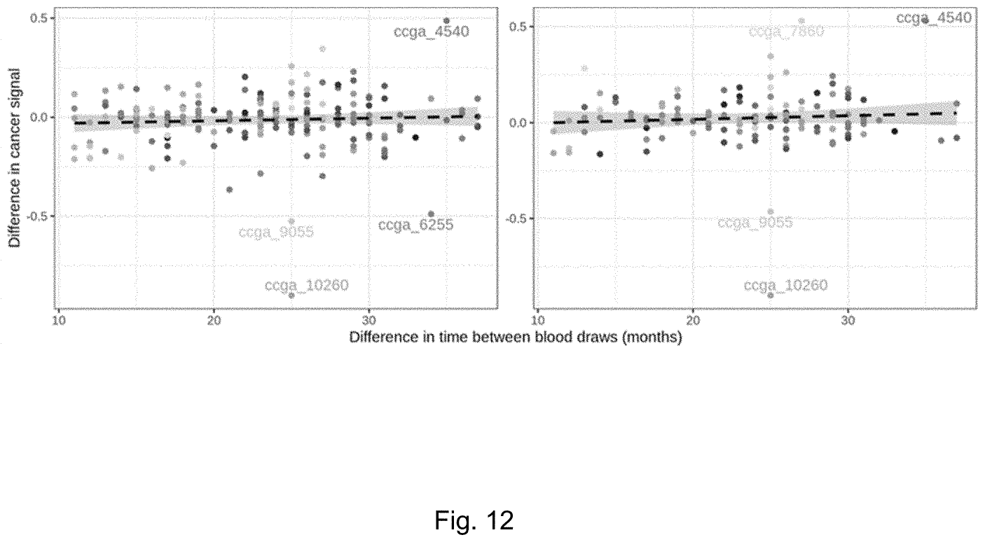

To investigate whether the time between the first blood draw and the second blood draw significantly affected cancer probabilities, each change in cancer probability score was plotted as a function of the time interval between the first and second blood draw. As shown in FIG. 12, no strong relationship is seen between the change in cancer probability scores and the passage of time within a short time-range of the longitudinal dataset.

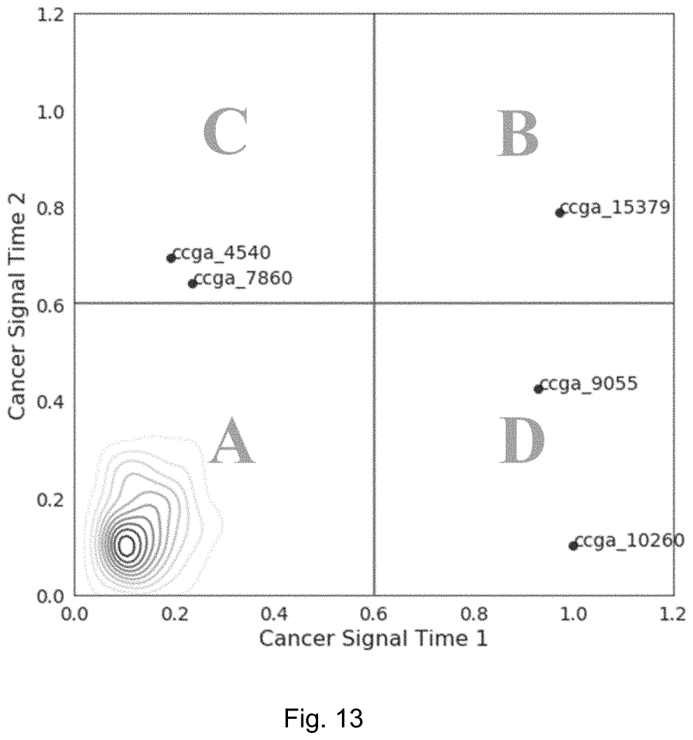

To investigate the biological significance underlying the large changes in cancer probability score, the medical record of several of the corresponding subjects was further investigated. These subjects correspond to the points falling outside of the lower left quadrant of the graph in FIG. 11, as represented again in FIG. 13. The density plot in FIG. 13 represents the distribution computed from the longitudinal participants, averaging over v1 and v2 assays at the initial blood draw.

The medical record for subject ccga_15379 was investigated. This subject fell within the upper right quadrant of the graph shown in FIGS. 11 and 13, indicating that a stable cancer signal was present in the first and second blood draws, taken twelve months apart for this subject. While this subject displayed no clinical indications of cancer, they were diagnosed with monoclonal gammopathy of undetermined significance (MGUS) more than 10 years prior to the first blood draw. MGUS is a condition caused by abnormal changes in plasma cells, which usually does not cause any symptoms. Approximately 1% of patients with MGUS develop blood cancer, such as multiple myeloma, each year.

The medical record for subjects ccga_4540 and ccga_7860 were also investigated. These subjects fell within the upper left quadrant of the graph shown in FIGS. 11 and 13, indicating that a significant cancer signal developed within these patients in the time between the first and second blood draws.

The medical record for subject ccga_4540 has no indication that this subject has developed cancer. However, the time between the first and second blood draws for this subject was 35 months, which is one of the longest time periods investigated. One possibility is that this observed change is due to a relationship between the passage of time and change in the cancer probability score for a subject. A second possibility is that this observed change is representative of a pre-cancerous or cancerous state that is not yet clinically detectable. A third possibility is that clinical records associated with the change are not available yet.

In contrast, the medical record for subject ccga_7860 shows that this subject was diagnosed with a bladder cancer within a month of the second blood draw. This indicates that the change in the cancer signal detected in the longitudinal blood draw, collected 27 months after the initial blood draw, represents cancer development in this subject.

The medical record for subjects ccga_10260 and ccga_9055 were also investigated. These subjects fell within the lower right quadrant of the graph shown in FIGS. 11 and 13, indicating that cancer signal detected in the first blood draw significantly diminished between the first and second blood draws.

The medical record for subject ccga_10260 shows at the time the initial blood draw was taken, the subject had not been diagnosed with cancer. However, three months later, this subject was diagnosed with ER+/PR+/HER2− breast cancer. Significantly, this is a slow growing, luminal cancer, suggesting that the subject had already developed the cancer at the time of the first blood draw. The subject was then treated by mastectomy after neoadjuvant therapy, followed by irradiation, prior to the second blood draw, which occurred 25 months after the initial blood draw. Significantly, this is a type of cancer typically associated with a positive clinical prognosis, which is consistent with the significant drop in cancer signal detected in the second blood draw.

The medical record for subject ccga_9055 indicates that the subject has displayed no clinical signs of cancer. However, subject ccga_9055 was diagnosed with MGUS and thrombocytopenia. While the cancer signal for subject ccga_9055 diminished within the 25 months between the first and second blood draws, the drop in signal was less than for subject ccga_10260. This is consistent with the results seen for subject ccga_15379, who was also diagnosed with MGUS, who observed a modest drop in signal over time. These results indicate that subjects with non-cancerous blood disorders, such as MGUS, may display a larger natural variance in their biological cancer signals.

A central hypothesis is that, beyond typical variation, a detected cancer signal only increases with time. To test this hypothesis two analyses will be investigated. First, whether positive cancer detected signals at baseline (initial blood draw) remain positive at the subsequent blood draw. Second, whether negative cancer signals at baseline convert to positive cancer signals detected at the later time point, or whether there is no detectable directionality of the signal. The analyses will be conducted using R software version 3.6 or higher.

To calculate classifier prediction transitions between the baseline and second blood draws, the following metrics will be computed. First, concordance of the classifier results (positive vs negative) between the participant-matched baseline and additional blood samples will be evaluated by constructing a 2×2 matrix and estimating positive percent agreement, negative percent agreement, overall agreement and the fraction of samples whose prediction changes from non-cancer to cancer between classifier results from the two blood draws.

Second, contribution of covariates to classifier prediction transitions will be estimated. An indicator variable representing whether a sample's cancer status changed between the two predictions will be calculated. A logistic regression model will then be fit using this indicator as the dependent variable and an additive model of sex, age-bin, and the number of months between the blood draws as covariates. Interaction effects between the covariates will also be included if there are enough samples that change in cancer prediction between the blood draws. It cannot be predicted how many samples will have a changing cancer signal between the blood draws. If less than 10 samples change in their cancer prediction this analysis will not be performed.

Third, a generalized linear mixed model will be fit with a binary outcome representing the classifier prediction and fixed effects using measured covariates, such as age and gender. A random effect whose covariance represents the “longitudinal” correlation induced by sampling the same participants at different time points will be modeled. For efficient computation this temporal covariance will be parameterized using a discrete autoregressive process model. If there is no variation in the cancer prediction between the blood draws, it will not be possible to fit this model or learn the underlying temporal covariance. As above, if less than 10 samples change in their cancer prediction, this analysis will not be performed.

Fourth, the latent difference in classifier probabilities (or logit-transformed probabilities) will be modeled as a two component mixture distribution, where the first component is a point-mass at zero and the second component is a flexible non-negative distribution. A Gaussian likelihood that allows for sampling variation in the observed difference in cancer probabilities will be used. This model captures the fact that most samples will have no change in their latent cancer probability, but some will shift towards increased cancer probability as time proceeds. The probability of belonging to either component will be estimated from the data using an empirical Bayes approach.

Fifth, the number of samples that received a different TOO call between the two blood draws including those with a “cancer not detected” assignment will be calculated. Among the samples that received a cancer TOO assignment, a “difference” metric (e.g., Kullback-Leibler divergence) between the fitted probabilities output by the TOO classifier for each sample between time points will be determined.

In addition, several exploratory analyses will be performed. First, a redaction analysis will be applied, using the first blood draw as baseline data whose signal would be removed from the second blood draw. Using this approach any fragments that look unusual with respect to the baseline can be removed, and the same analyses as above can be re-run with the redacted data.

Second, a set of methylation variants will be defined using a large reference database of non-cancer WGBS cfDNA samples from CCGA1 (e.g., that do not overlap with the participants analyzed in this study) and fully methylated or unmethylated variants that are rare in non-cancer samples will be filtered. The reference set will be locked in advance of analyzing the follow-up samples. The data set will be conditioned on a high probability of cancer, and test performed for a shift distribution of frequency change between time-points, where the shift represents a potential increase in the underlying tumor fraction.

Third, the subset of samples that have received a tissue of origin (TOO) call at the first blood draw will be focused on. For each predicted tissue of origin in the first time point, target methylation variants will be defined from a pre-computed reference database of methylation variants called on that corresponding TOO, filtering variants that are high frequency in the database. The posterior distribution of tumor fraction will then be estimated and a potential shift in tumor fraction between the first and second blood draw will be inferred/tested for. The same “reference free” tumor fraction estimation approach described above will then be performed, but conditioned on the TOO call at the second blood draw, rather than the first.

Fourth, Uniform Manifold Approximation and Projection (UMAP) and Principal Component Analysis (PCA) will be applied to the mixture model feature matrix generated for the longitudinal pilot data. Each row of this matrix will represent a sample and each column will represent a mixture model feature. Notably, the same individual will be present in different rows but their data being sampled at different blood draws. We will then regress a number of covariates (age, sex, assay-type, blood draw indicator) on each dimension output from UMAP to gain interpretation into what patterns drive similarities among the samples.

Fifth, Principal Component Analysis (PCA) will be applied to the mixture model features generated for the training set samples. Each longitudinal pilot data sample will then be projected onto the axes of variation defined by the PCA applied to the training set. This will allow leverage of the large and diverse collection of samples from the training set to look for overall relationships among samples from the smaller longitudinal pilot data. Similar regression of the same covariates from above will be performed to look for associations.

### CONCLUSION

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that comprises a computer program mechanism embedded in a non-transitory computer readable storage medium. For instance, the computer program product could contain the program modules shown and/or described in any combination of FIGS. 1-8. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

1. A method of determining whether a test subject has a disease condition comprising:
at a computer system having one or more processors, and memory storing one or more programs for execution by the one or more processors:
(A) determining, from a first plurality of sequence reads, in electronic form, the first plurality of sequence reads comprising hundreds of thousands of sequence reads of a first plurality of nucleic acid molecules in a first biological sample obtained from the test subject at a first test time point, a first genotypic data construct for the test subject, the first genotypic data construct comprising values for a plurality of genotypic characteristics based on the first plurality of sequence reads;
(B) inputting the first genotypic data construct into a model for the disease condition, thereby generating a first model score set for the disease condition;
(C) determining, from a second plurality of sequence reads, in electronic form, the second plurality of sequence reads comprising hundreds of thousands of sequence reads of a second plurality of nucleic acid molecules in a second biological sample obtained from the test subject at a second test time point, a second genotypic data construct for the test subject, the second genotypic data construct comprising values for the plurality of genotypic characteristics based on the second plurality of sequence reads;
(D) inputting the second genotypic data construct into the model, thereby generating a second model score set for the disease condition;
(E) determining a test delta score set based on a difference between the first and second model score set; and
(F) evaluating the test delta score set against a plurality of reference delta score sets, thereby determining whether the test subject has the disease condition, wherein each reference delta score set in the plurality of reference delta scores sets is for a respective reference subject in a plurality of reference subjects.

2. (canceled)

3. The method of claim 1, wherein:
the first model score set comprises a probability that the test subject has the disease condition at the first test time point,
the second model score set comprises of a probability that the test subject has the disease at the second test time point, and
the evaluating (F) comprises comparing the test delta score set to a distribution of the reference delta score sets, wherein each reference delta score set in the plurality of reference delta score sets is for a respective reference subject in the plurality of reference subject based on a difference between:
(i) a first probability that the respective reference subject has the disease condition provided by the model using a respective first reference genotypic data construct comprising values for the plurality of genotypic features, taken using a respective first biological sample acquired at a respective first time point from the respective reference subject, and
(ii) a second probability that the respective reference subject has the disease condition provided by the model using a respective second genotypic data construct comprising values for the plurality of genotypic features, taken using a respective second biological sample acquired from the respective reference subject at a respective second time point occurring after the respective first time point, and wherein the respective training subject is free of the disease condition during at least the respective first and second time points.

4. (canceled)

5. (canceled)

6. The method of claim 3, wherein the evaluating (F) comprises:
determining a measure of central tendency of the distribution and a measure of spread of the distribution, and
determining a significance of the test delta score set using the measure of central tendency of the distribution and the measure of spread of the distribution.

7. (canceled)

8. (canceled)

9. The method of claim 6, wherein:
the measure of central tendency of the distribution is the mean of the distribution,
the measure of spread of the distribution is the standard deviation of the distribution, and
the determining the significance of the test delta score set using the measure of central tendency of the distribution and the measure of spread of the distribution comprises determining a number of standard deviations the test delta score set is from the mean of the distribution.

10. (canceled)

11. The method of claim 1, wherein the evaluating (F) comprises inputting the test delta score into a classifier trained against the plurality of reference delta score sets, wherein each reference delta score set in the plurality of reference delta score sets is for a respective reference subject in the plurality of reference subject based on a difference between:
(i) a first probability that the respective reference subject has the disease condition provided by the model using a respective first reference genotypic data construct comprising values for the plurality of genotypic features, taken using a respective first biological sample acquired at a respective first time point from the respective reference subject, and
(ii) a second probability that the respective reference subject has the disease condition provided by the model using a respective second genotypic data construct comprising values for the plurality of genotypic features, taken using a respective second biological sample acquired from the respective reference subject at a respective second time point occurring after the respective first time point, and wherein the respective training subject is free of the disease condition during at least the respective first and second time points.

12. The method of claim 11, wherein the classifier is further trained against, for each respective training subject in at least a subset of the plurality of reference subjects, a determination of whether the respective subject had the disease condition at a respective third time point occurring after the respective second time point.

13. (canceled)

14. The method of claim 3, wherein:
an amount of time between the respective first time point and the respective second time point for each respective reference subject in the plurality of reference subjects is used as a covariate in calculating the distribution; and
the test delta score set is adjusted based on the covariate representing a difference in time between the first test time point and the second test time point for the test subject.

15. (canceled)

16. (canceled)

17. The method of claim 3, further comprising normalizing, wherein:
each respective reference delta score set in the plurality of reference delta scores sets is normalized for an amount of time between the respective first time point and the respective second time point for the respective subject; and
the test delta score set is normalized for an amount of time between the first test time point and the second test time point.

18. (canceled)

19. (canceled)

20. The method of claim 3, wherein:
A non-genotypic characteristic of each respective reference subject in the plurality of reference subjects is used as a covariate in calculating the distribution; and
the test delta score set is adjusted based on the non-genotypic characteristic of the test subject.

21. (canceled)

22. (canceled)

23. The method of claim 1, further comprising a normalizing, wherein:
each respective reference delta score set in the plurality of reference delta score sets is normalized for a non-genotypic characteristic of the respective reference subject; and
the test delta score set is normalized for the non-genotypic characteristic of the test subject.

24. (canceled)

25. (canceled)

26. The method of claim 20, wherein the non-genotypic characteristic comprises age, alcohol consumption status, smoking status, gender, or a combination thereof.

27. (canceled)

28. The method of claim 1, wherein the disease condition is a cancer condition.

29. The method of claim 1, wherein the disease condition is a type of disease condition in a set of disease conditions and the model provides a probability or likelihood for each disease condition in the set conditions.

30-35. (canceled)

36. The method of claim 1, wherein the disease condition is a coronary disease.

37-39. (canceled)

40. The method of claim 1, wherein the first biological sample obtained from the test subject and the second biological sample obtained from the test subject are both blood samples.

41. (canceled)

42. (canceled)

43. The method of claim 1, wherein the first plurality of nucleic acid molecules and the second plurality of nucleic acid molecules are cell-free DNA molecules.

44. (canceled)

45. The method of claim 1, wherein the plurality of genotypic characteristics comprises a plurality of relative copy numbers, wherein each respective relative copy number in the plurality of relative copy numbers corresponds to a different genetic location in a plurality of genetic locations.

46. The method of claim 1, wherein the plurality of genotypic characteristics comprises a plurality of methylation statuses, wherein each methylation status in the plurality of methylation statuses corresponds to a different genetic location in a plurality of genetic locations.

47. The method of claim 1, wherein:
the plurality of genotypic characteristics for the first genotypic data structure comprises a first plurality of bin values, each respective bin value in the first plurality of bin values representing a corresponding bin in a plurality of bins,
each respective bin value in the first plurality of bin values is representative of a number of unique nucleic acid fragments with a predetermined methylation pattern identified using sequence reads in the first plurality of sequence reads that map to the corresponding bin in the plurality of bins,
the plurality of genotypic characteristics for the second genotypic data structure comprises a second plurality of bin values, each respective bin value in the second plurality of bin values representing a corresponding bin in the plurality of bins,
each respective bin value in the second plurality of bin values is representative of a number of unique nucleic acid fragments with a predetermined methylation pattern identified using sequence reads in the second plurality of sequence reads that map to the corresponding bin in the plurality of bins, and
each bin in the plurality of bins represents a non-overlapping region of a reference genome of a species of the test subject.

48-60. (canceled)

61. The method of claim 1, wherein the model is trained on a cohort of subjects in which a first portion of the cohort has the disease condition and a second portion of the cohort is free of the disease condition.

62. (canceled)

63. The method of claim 1, wherein the model comprises:
(i) an input layer for receiving values for the plurality of genotypic characteristics, wherein the plurality of genotypic characteristics comprises a first number of dimensions, and
(ii) an embedding layer that comprises a set of weights, wherein the embedding layer directly or indirectly receives output of the input layer, and wherein an output of the embedding layer is a model score set having a second number of dimensions that is less than the first number of dimensions, and
(iii) an output layer that directly or indirectly receives the model score set from the embedding layer, wherein,
the first model score set is the model score set of the embedding layer upon inputting the first genotypic data construct into the input layer, and
the second model score set is the model score set of the embedding layer upon inputting the second genotypic data construct into the input layer.

64. The method of claim 63, wherein:
the evaluating (F) comprises evaluating the test delta score set using a logistic function trained by logistic regression against the plurality of reference delta score sets, wherein each reference delta score set in the plurality of reference delta scores is for a respective reference subject in the plurality of reference subjects based on a difference between:
(i) a first score set provided by the embedding layer of the model using a first respective reference genotypic data construct comprising values for the plurality of genotypic features, taken using a first respective biological sample acquired at a respective first time point from the respective reference subject, and
(ii) a second score set provided by the embedding layer of the model using a second respective genotypic data construct comprising values for the plurality of genotypic features, taken using a second respective biological sample acquired from the respective reference subject at a respective second time point other than the first respective time point.

65-67. (canceled)

68. The method of claim 1, further comprising, prior to evaluating (F):
determining a plurality of baseline genotypic data constructs for the test subject, each respective baseline genotypic data construct in the plurality of baseline genotypic data constructs comprising values for the plurality of genotypic characteristics based on a corresponding baseline plurality of sequence reads, in electronic form, of a corresponding plurality of nucleic acid molecules in a corresponding baseline biological sample, in a plurality of baseline biological samples, obtained from the test subject at a corresponding baseline test time point occurring before the second test time point;
using an amount of variance in values for one or more respective genotypic characteristic, in the plurality of genotypic characteristics, between respective baseline genotypic data constructs in the plurality of baseline genotypic constructs to calculate a baseline variance covariate specific to the test subject; and
applying the baseline covariate to the distribution of the reference delta score sets, to normalize the distribution of the reference delta score sets against the baseline variability of the test subject.

69. The method of claim 1, wherein a span between the first test time point and the second test time point is based upon the first model score set.

70. A non-transitory computer-readable storage medium having stored thereon program code instructions that, when executed by a processor, cause the processor to perform the method of claim 1.

71. A computer system comprising:
one or more processors; and
a non-transitory computer-readable medium including computer-executable instructions that, when executed by the one or more processors, cause the processors to perform a method of claim 1.

72. A method of determining whether a test subject has a disease condition comprising:
at a computer system having one or more processors, and memory storing one or more programs for execution by the one or more processors:
(A) for each respective test time point in a plurality of test time points:
(i) determining a corresponding genotypic data construct for the test subject, the corresponding genotypic data construct comprising values for a plurality of genotypic characteristics based on a corresponding plurality of sequence reads, in electronic form, of a corresponding plurality of nucleic acid molecules in a corresponding biological sample obtained from the test subject at the respective test time point, and
(ii) inputting the corresponding genotypic data construct into a model for the disease condition, thereby generating a corresponding time stamped model score set for the disease condition at the respective test time point;
thereby obtaining a plurality of time stamped test model score sets for the test subject, each respective time stamped test model score set coupled to a different test time point in the plurality of test time points;
(B) fitting the plurality of time stamped test model score sets with a temporal trend test thereby obtaining a test trend parameter set for the test subject; and
(C) evaluating the test trend parameter set for the test subject against a plurality of reference trend parameter sets for a plurality of reference subjects thereby determining the disease condition of the test subject, wherein each respective reference trend parameter set in the plurality of reference trend parameter sets is for a corresponding reference subject in the plurality of reference subjects.

73-131. (canceled)
